(19) United States
(12) Patent Application Publication
(10) Pub. No.: US 2025/0250556 A1
(43) Pub. Date: Aug. 7, 2025

(54) Polypeptides Having Peptidoglycan Degrading Activity and Polynucleotides Encoding Same

(71) Applicant: Novozymes A/S, Bagsvaerd (DK)

(72) Inventors: Christian Berg Oehlenschlaeger, Valby (DK); Dorotea Raventos Segura, Runsted (DK); Jesper Salomon, Holte (DK); Fabian Barrientos Garcia, Birkerod (DK); Lillian Eva Tang Baltsen, Bagsvaerd (DK); Christian Bech Rosen, Kobenhavn (DK)

(73) Assignee: Novozymes A/S, Bagsvaerd (DK)

(21) Appl. No.: 19/095,734

(22) Filed: Mar. 31, 2025

Related U.S. Application Data
(62) Division of application No. 18/586,709, filed on Feb. 26, 2024, now Pat. No. 12,297,470.
Application No. 18/586,709 is a division of application No. 17/298,836, filed on Jun. 1, 2021, now Pat. No. 11,959,111.

(30) Foreign Application Priority Data
Dec. 21, 2018 (EP) 18215408.8

(51) Int. Cl. C12N 9/80; C11D 3/386

(52) U.S. Cl. CPC C12N 9/80; C11D 3/38636; C12Y 305/01028

(57) ABSTRACT

The present invention relates to cleaning compositions comprising polypeptides having peptidoglycan degradation activity, as well as use of the cleaning compositions for cleaning of an item such as a textile or a surface.

### CROSS-REFERENCE TO RELATED APPLICATIONS

This application is a division of U.S. application Ser. No. 18/586,709 filed Feb. 26, 2024, now pending, which is a division of U.S. application Ser. No. 17/298,836 filed on Jun. 1, 2021, now U.S. Pat. No. 11,959,111, which is a 35 U.S.C. 371 national application of international application no. PCT/EP2019/086399 filed Dec. 19, 2019, which claims priority or the benefit under 35 U.S.C. 119 of EP application no. 18215408.8 filed Dec. 21, 2018. The disclosure of each application is fully incorporated herein by reference.

### REFERENCE TO A SEQUENCE LISTING

This application contains a Sequence Listing in computer readable form, which is incorporated herein by reference. The Sequence Listing was created on Mar. 7, 2025 and is named SQ.xml and is 193,658 bytes in size.

### BACKGROUND OF THE INVENTION

### Field of the Invention

The present invention relates to polypeptides having peptidoglycan degrading activity, polynucleotides encoding the polypeptides and catalytic domains belonging to peptidoglycan degrading enzyme families. The invention further relates to compositions comprising such polypeptides, in particular cleaning compositions, use of polypeptides having peptidoglycan degrading activity in cleaning processes and/or for removal or reduction of bacterial-derived peptidoglycan, and methods for removal or reduction of peptidoglycan. The invention further relates to nucleic acid constructs, vectors, and host cells comprising polynucleotides encoding the polypeptides as well as methods of producing and using the polypeptides and catalytic domains.

### Description of the Related Art

Enzymes have been used in detergents for decades. Usually, a cocktail of various enzymes is added to detergent compositions. The enzyme cocktail often comprises various enzymes, wherein each enzyme targets a specific substrate, e.g., amylases are active towards starch stains, proteases on protein stains and so forth. Textiles and surfaces such as laundry and dishes become soiled with many different types of soiling. The soiling may be composed of proteins, grease, starch etc. Complex stains composed of different organic materials such as food stains, sebum, dead cell material, EPS (extracellular polymeric matrix) from, e.g., biofilm are difficult to remove completely with traditional ADW (automatic dishwashing) and laundry detergent compositions. Contributing to the organic matter is peptidoglycan, originating from the bacterial cell wall. Bacteria are present in high numbers in laundry items. When the bacteria lyse, the destroyed cells leave a high amount of cell wall-derived peptidoglycan in the textile or on hard surfaces such as the inner surfaces of a washing machine. This peptidoglycan substrate may be sticky or gluing, which when present on textile attracts soils and may cause redeposition or backstaining of soil, resulting in a greying of the textile. Also, malodors from, e.g., sweat, cigarette smoke and pollution are particularly difficult to remove from, e.g., textiles. Malodor is a growing problem, particularly in laundry, with the changed habits of lower temperature washing, front loading wash machines that save water but leave behind residual water between loads, thus allowing bacterial biofilms to flourish, line drying clothes to save energy rather than appliance drying, and the increased popularity of synthetic fabrics, such as athletic wear, that appear to retain odors more than natural fabrics. In conventional detergent compositions such as laundry detergents the above problems are often solved by adding perfumes. This solution is not completely effective, however, as it is short term and furthermore only serves to mask malodor rather than dealing with the underlying cause of malodor. There is thus a need in the art for new solutions for overcoming the problems of malodor and redeposition.

### SUMMARY OF THE INVENTION

The invention relates to a cleaning composition comprising a peptidoglycan degradation enzyme, at least one surfactant and at least one additional cleaning component selected from builders and bleach components. The cleaning composition may, e.g., comprise a peptidoglycan degradation enzyme, at least 5 wt % anionic surfactants, and at least one additional cleaning component selected from at least one builder and at least one bleach component.

The invention further relates to the use of such a composition for cleaning of an item such as a textile or a surface. The invention further relates to a method of cleaning an item, comprising the steps of:
a) contacting the item with a solution comprising a peptidoglycan degradation enzyme having peptidoglycan lyase activity and preferably N-acetylmuramyl-L-alanine amidase activity; and a cleaning component, wherein the cleaning component is selected from 5 to 60 wt % of at least one surfactant; 5 to 50 wt % of at least one builder; and 1 to 20 wt % of at least one bleach component; and optionally
b) rinsing the item,
   wherein the item is preferably a textile.

### Overview of Sequences

SEQ ID NO: 1 DNA encoding full length polypeptide from Hamadaea tsunoensis
SEQ ID NO: 2 polypeptide derived from SEQ ID NO: 1
SEQ ID NO: 3 mature polypeptide obtained from Hamadaea tsunoensis
SEQ ID NO: 4 DNA encoding full length polypeptide from Micromonospora maritima
SEQ ID NO: 5 polypeptide derived from SEQ ID NO: 4
SEQ ID NO: 6 mature polypeptide obtained from Micromonospora maritma
SEQ ID NO: 7 DNA encoding full length polypeptide from Paenibacillus sp.
SEQ ID NO: 8 polypeptide derived from SEQ ID NO: 7
SEQ ID NO: 9 mature polypeptide obtained from Paenibacillus sp.
SEQ ID NO: 10 DNA encoding full length polypeptide from Nonomuraea sp.
SEQ ID NO: 11 polypeptide derived from SEQ ID NO: 10
SEQ ID NO: 12 mature polypeptide obtained from Nonomuraea sp.
SEQ ID NO: 13 DNA encoding full length polypeptide from Lysobacter antibioticus
SEQ ID NO: 14 polypeptide derived from SEQ ID NO: 13
SEQ ID NO: 15 mature polypeptide obtained from Lysobacter antibioticus
SEQ ID NO: 16 DNA encoding full length polypeptide from Micromonospora sp.
SEQ ID NO: 17 polypeptide derived from SEQ ID NO: 16
SEQ ID NO: 18 mature polypeptide obtained from Micromonospora sp.
SEQ ID NO: 19 DNA encoding full length polypeptide from Nonomuraea coxensis
SEQ ID NO: 20 polypeptide derived from SEQ ID NO: 19
SEQ ID NO: 21 mature polypeptide obtained from Nonomuraea coxensis
SEQ ID NO: 22 DNA encoding full length polypeptide from Micromonospora fulvopurpurea
SEQ ID NO: 23 polypeptide derived from SEQ ID NO: 22
SEQ ID NO: 24 mature polypeptide obtained from Micromonospora fulvopurpurea
SEQ ID NO: 25 DNA encoding full length polypeptide from Alicyclobacillus sp.
SEQ ID NO: 26 polypeptide derived from SEQ ID NO: 25
SEQ ID NO: 27 mature polypeptide obtained from Alicyclobacllus sp.
SEQ ID NO: 28 DNA encoding full length polypeptide from Halomonas sp.
SEQ ID NO: 29 polypeptide derived from SEQ ID NO: 28
SEQ ID NO: 30 mature polypeptide obtained from Halomonas sp.
SEQ ID NO: 31 DNA encoding full length polypeptide from Pseudomonas peli
SEQ ID NO: 32 polypeptide derived from SEQ ID NO: 31
SEQ ID NO: 33 mature polypeptide obtained from Pseudomonas peli
SEQ ID NO: 34 DNA encoding full length polypeptide from Halomonas sp.
SEQ ID NO: 35 polypeptide derived from SEQ ID NO: 34
SEQ ID NO: 36 mature polypeptide obtained from Halomonas sp.
SEQ ID NO: 37 DNA encoding full length polypeptide from Pseudomonas pseudoalcaligenes
SEQ ID NO: 38 polypeptide derived from SEQ ID NO: 37
SEQ ID NO: 39 mature polypeptide obtained from Pseudomonas pseudoalcaligenes
SEQ ID NO: 40 DNA encoding full length polypeptide from Tumebacillus sp.
SEQ ID NO: 41 polypeptide derived from SEQ ID NO: 40
SEQ ID NO: 42 mature polypeptide obtained from Tumebacillus sp.
SEQ ID NO: 43 DNA encoding full length polypeptide from Nonomuraea dietziae
SEQ ID NO: 44 polypeptide derived from SEQ ID NO: 43
SEQ ID NO: 45 mature polypeptide obtained from Nonomuraea dietziae
SEQ ID NO: 46 DNA encoding full length polypeptide from Laceyella sacchari
SEQ ID NO: 47 polypeptide derived from SEQ ID NO: 46
SEQ ID NO: 48 mature polypeptide obtained from Laceyella sacchari
SEQ ID NO: 49 DNA encoding full length polypeptide from Thermostaphylospora chromogena
SEQ ID NO: 50 polypeptide derived from SEQ ID NO: 49
SEQ ID NO: 51 mature polypeptide obtained from Thermostaphylospora chromogena
SEQ ID NO: 52 DNA encoding full length polypeptide from Kribbella aluminosa
SEQ ID NO: 53 polypeptide derived from SEQ ID NO: 52
SEQ ID NO: 54 mature polypeptide obtained from Kribbella aluminosa
SEQ ID NO: 55 DNA encoding full length polypeptide from Streptomyces griseus
SEQ ID NO: 56 polypeptide derived from SEQ ID NO: 55
SEQ ID NO: 57 mature polypeptide obtained from Streptomyces griseus
SEQ ID NO: 58 DNA encoding full length polypeptide from Micromonospora peucetia
SEQ ID NO: 59 polypeptide derived from SEQ ID NO: 58
SEQ ID NO: 60 mature polypeptide obtained from Micromonospora peucetia
SEQ ID NO: 61 DNA encoding full length polypeptide from Bacillus sp.
SEQ ID NO: 62 polypeptide derived from SEQ ID NO: 61
SEQ ID NO: 63 mature polypeptide obtained from Bacillus sp.
SEQ ID NO: 64 DNA encoding full length polypeptide from Bacillus sporothermodurans
SEQ ID NO: 65 polypeptide derived from SEQ ID NO: 64
SEQ ID NO: 66 mature polypeptide obtained from Bacillus sporothermodurans
SEQ ID NO: 67 DNA encoding full length polypeptide from Paenibacillus pini
SEQ ID NO: 68 polypeptide derived from SEQ ID NO: 67
SEQ ID NO: 69 mature polypeptide obtained from Paenibacillus pini
SEQ ID NO: 70 DNA encoding full length polypeptide from Bacillus cohnii
SEQ ID NO: 71 polypeptide derived from SEQ ID NO: 70
SEQ ID NO: 72 mature polypeptide obtained from Bacillus cohnii
SEQ ID NO: 73 DNA encoding full length polypeptide from Kribbella sp.
SEQ ID NO: 74 polypeptide derived from SEQ ID NO: 73
SEQ ID NO: 75 mature polypeptide obtained from Kribbella sp.
SEQ ID NO: 76 DNA encoding full length polypeptide from Bacillus sp.
SEQ ID NO: 77 polypeptide derived from SEQ ID NO: 76
SEQ ID NO: 78 mature polypeptide obtained from Bacillus sp.
SEQ ID NO: 79 DNA encoding full length polypeptide from Bacillus sp.
SEQ ID NO: 80 polypeptide derived from SEQ ID NO: 79
SEQ ID NO: 81 mature polypeptide obtained from Bacillus sp.
SEQ ID NO: 82 DNA encoding full length polypeptide from Bacillus sp.
SEQ ID NO: 83 polypeptide derived from SEQ ID NO: 82
SEQ ID NO: 84 mature polypeptide obtained from Bacillus sp.
SEQ ID NO: 85 DNA encoding full length polypeptide from Streptomyces sp.
SEQ ID NO: 86 polypeptide derived from SEQ ID NO: 85
SEQ ID NO: 87 mature polypeptide obtained from Streptomyces sp.
SEQ ID NO: 88 DNA encoding full length polypeptide from Bacillus sp.
SEQ ID NO: 89 polypeptide derived from SEQ ID NO: 88
SEQ ID NO: 90 mature polypeptide obtained from Bacillus sp.
SEQ ID NO: 91 DNA encoding full length polypeptide from Bacillus sp.
SEQ ID NO: 92 polypeptide derived from SEQ ID NO: 91
SEQ ID NO: 93 mature polypeptide obtained from Bacillus sp.
SEQ ID NO: 94 DNA encoding full length polypeptide from Nonomuraea guangzhouensis
SEQ ID NO: 95 polypeptide derived from SEQ ID NO: 94
SEQ ID NO: 96 mature polypeptide obtained from Nonomuraea guangzhouensis
SEQ ID NO: 97 DNA encoding full length polypeptide from Nonomuraea guangzhouensis
SEQ ID NO: 98 polypeptide derived from SEQ ID NO: 97
SEQ ID NO: 99 mature polypeptide obtained from Nonomuraea guangzhouensis
SEQ ID NO: 100 DNA encoding full length polypeptide from Bacillus cohnii
SEQ ID NO: 101 polypeptide derived from SEQ ID NO: 100
SEQ ID NO: 102 mature polypeptide obtained from Bacillus cohnii
SEQ ID NO: 103 DNA encoding full length polypeptide from Halomonas sp.
SEQ ID NO: 104 polypeptide derived from SEQ ID NO: 103
SEQ ID NO: 105 mature polypeptide obtained from Halomonas sp.
SEQ ID NO: 106 DNA encoding full length polypeptide from Lysobacter capsici
SEQ ID NO: 107 polypeptide derived from SEQ ID NO: 106
SEQ ID NO: 108 mature polypeptide obtained from Lysobacter capsica
SEQ ID NO: 109 MKKPLGKIVASTALLISVAFSSSIASA (signal peptide)

### SEQ ID NO: 110 HHHHHHPR (His-tag)

### SEQ ID NO: 111 Motif

### Definitions

Peptidoglycan degrading enzymes: The term “peptidoglycan degrading enzyme” means an enzyme having activity towards peptidoglycan. Peptidoglycan (PGN) is a major component of the bacterial cell envelope in both Gram-positive and Gram-negative bacteria (Human et al., 2009, J. Innate Immun. 1: 88-97). The peptidoglycan structure of both Gram-positive and Gram-negative bacteria comprises repeating disaccharide backbones of N-acetylglucosamine (NAG) and β-(1-4)-N-acetylmuramic acid (NAM) that are cross-linked by peptide stem chains attached to the NAM residues (Bourhis et al., 2007, Microbes Infect. 9(5): 629-636). The peptide and glycopeptide fragments of PGN are commonly referred to as “muropeptides.” PGN hydrolases are defined by their catalytic specificities. Two classes of these enzymes digest the PGN glycan backbone, N-acetylmuramidases which cleave PGN between the NAG-NAM bond upstream of NAM and N-acetylglucosaminidases which cleave the NAM-NAG bond. In contrast, N-acetylmuramyl-L-alanine amidases cleave between NAM and the first alanine of the peptide chain. Thus, catalysis by N-acetylmuramyl-L-alanine amidases separate the PGN sugar backbones from the stem peptide chain (Fournier et al., 2005, Clin. Microbiol. Rev. 18(3): 521-540). The enzymes of the invention comprise an N-acetylmuramyl-L-alanine amidase (EC 3.5.1.28) domain. In the context of the present invention, N-acetylmuramyl-L-alanine amidases may also be termed peptidoglycan amidohydrolases. The enzymes of the invention comprise in addition to the amidase domain also a peptidoglycan lyase domain (GH23-like). The GH 23 family comprises lysozyme type G (EC 3.2.1.17), peptidoglycan lyase (EC 4.2.2.n1, peptidoglycan lytic exotransglycosylase, and 4.2.2.n2, peptidoglycan lytic endotransglycosylase) and chitinases (EC 3.2.1.14). The domain comprised by the enzymes of the invention is a peptidoglycan lyase domain (EC 4.2.2.n1 or 4.2.2.n2). Peptidoglycan lyases are also termed lytic transglycosylases. Peptidoglycan lyases of GH23 constitute Family 1 of the organizational scheme of Blackburn and Clarke (Blackburn et al., 2001, J. Mol. Evol. 52(1): 78-84). The enzymes of this family cleave the β-1,4-linkage between N-acetylmuramyl and N-acetylglucosaminyl residues in peptidoglycan. However, unlike lysozyme, peptidoglycan lyases are not hydrolases but rather catalyze an intramolecular transglycosylation to the C-6 hydroxyl group of the muramyl residue, leading to the generation of a terminal 1,6-anhydromuramic acid product that is an acetal, and not a hemiacetal (Höltje, 1975, J. Bacteriol. 124(3):1067-1076. The enzymes of the invention are thus distinct from lysozymes.

The enzymes of the invention preferably comprise an N-acetylmuramyl-L-alanine amidase (EC 3.5.1.28) domain as well as a peptidoglycan lyase domain (EC 4.2.2.n1 or 4.2.2.n2). Thus, in the present invention peptidoglycan degrading enzymes are preferably N-acetylmuramyl-L-alanine amidases (EC 3.5.1.28) and peptidoglycan lyases (EC 4.2.2.n1 or 4.2.2.n2) having amidase and lyase activity towards peptidoglycan.

For purposes of the present invention, peptidoglycan lyase activity and N-acetylmuramyl-L-alanine amidase activity may be determined according to the procedures described below in the example section.

The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

A biofilm is organic matter produced by any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single cells that may float or swim in a liquid medium. Bacteria living in a biofilm usually have significantly different properties from planktonic bacteria of the same species, as the dense and protected environment of the film allows them to cooperate and interact in various ways. One benefit of this environment for the microorganisms is increased resistance to detergents and antibiotics, as the dense extracellular matrix and the outer layer of cells protect the interior of the community. The biofilm living bacteria do not lose their ability to live as planktonic cells if the biofilm matrix is compromised. On laundry, biofilm- or EPS-producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp. In one aspect, the biofilm- or EPS-producing strain is Pseudomonas, for example Pseudomonas aeruginosa, Pseudomonas alcaliphila or Pseudomonas fluorescens.

The term “catalytic domain” means the region of an enzyme containing the catalytic machinery of the enzyme.

The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “clade” means a group of polypeptides clustered together on the basis of homologous features traced to a common ancestor. Polypeptide clades can be visualized as phylogenetic trees and a clade is a group of polypeptides that consists of a common ancestor and all its lineal descendants. Example 6 describes the generation of phylogenetic trees.

The term “coding sequence” means a polynucleotide which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

The term “cleaning component” means, e.g., a detergent adjunct ingredient that is different from the polypeptides of this invention. The precise nature of these additional cleaning or adjunct components, and levels of incorporation thereof, will depend on the physical form of the composition and the nature of the operation for which it is to be used. Suitable cleaning components include, but are not limited to the components described below, such as surfactants, builders and co-builders, flocculating aid, chelating agents, dye transfer inhibitors, enzymes (other than the enzymes of the invention), enzyme stabilizers, enzyme inhibitors, catalytic materials, bleach activators, hydrogen peroxide, sources of hydrogen peroxide, preformed peracids, polymeric agents, clay soil removal/anti-redeposition agents, brighteners, suds suppressors, dyes, perfumes, structure elasticizing agents, fabric softeners, carriers, hydrotropes, fabric hueing agents, anti-foaming agents, dispersants, processing aids, and/or pigments.

The term “cleaning composition” includes “detergent composition” and refers to compositions that find use in the removal of undesired compounds from items to be cleaned, such as textiles. The detergent composition may be used to, e.g., clean textiles for both household cleaning and industrial cleaning. The term encompasses any materials/compounds selected for the particular type of cleaning composition desired and the form of the product (e.g., liquid, gel, powder, granulate, paste, or spray compositions) and includes, but is not limited to, detergent compositions such as liquid and/or solid laundry detergents and fine fabric detergents; fabric fresheners; fabric softeners; and textile and laundry pre-spotters/pretreatment. In addition to containing the enzyme of the invention, the detergent formulation may contain one or more additional enzymes (such as proteases, amylases, lipases, cutinases, cellulases, endoglucanases, xyloglucanases, pectinases, pectin lyases, xanthanases, peroxidases, haloperoxygenases, catalases, mannanases, nucleases or any mixture thereof), and/or detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxidoreductases, bluing agents and fluorescent dyes, antioxidants, and solubilizers.

The term “expression” includes any step involved in the production of a polypeptide including, but not limited to, transcription, post-transcriptional modification, translation, post-translational modification, and secretion.

The term “expression vector” means a linear or circular DNA molecule that comprises a polynucleotide encoding a polypeptide and is operably linked to control sequences that provide for its expression.

The term “fragment” means a polypeptide having one or more amino acids absent from the amino and/or carboxyl terminus of a mature polypeptide or domain; wherein the fragment has peptidoglycan degradation activity.

The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g., a host cell may be genetically modified to express the polypeptide of the invention. The fermentation broth from that host cell will comprise the isolated polypeptide. It will be apparent to persons skilled in the art that the polypeptides disclosed herein are preferably in isolated form.

The term “laundering” relates to both household laundering and industrial laundering and means the process of treating textiles with a solution containing a cleaning or detergent composition of the present invention. The laundering process can for example be carried out using, e.g., a household or an industrial washing machine or can be carried out by hand.

The term “malodor” means an odor which is not desired on clean items. The cleaned item should smell fresh and clean without malodors adhered to the item. One example of malodor is compounds with an unpleasant smell, which may be produced by microorganisms. Another example is unpleasant smells which can be sweat or body odor adhered to an item which has been in contact with human or animal. Another example of malodor can be the odor from spices, which sticks to items for example curry or other spices which smell strongly.

The term “mature polypeptide” means a polypeptide in its mature form following N terminal processing (e.g., removal of signal peptide).

In one aspect, the mature polypeptide is amino acids 1 to 431 of SEQ ID NO: 2. Amino acids −29 to −1 of SEQ ID NO: 2 are a signal peptide. In one aspect, the mature polypeptide is amino acids 1 to 485 of SEQ ID NO: 5. Amino acids −30 to −1 of SEQ ID NO: 5 are a signal peptide.

In one aspect, the mature polypeptide is amino acids 1 to 483 of SEQ ID NO: 8. Amino acids −26 to −1 of SEQ ID NO: 8 are a signal peptide. In one aspect, the mature polypeptide is amino acids 1 to 471 of SEQ ID NO: 11. Amino acids −22 to −1 of SEQ ID NO: 11 are a signal peptide. In one aspect, the mature polypeptide is amino acids 1 to 639 of SEQ ID NO: 14. In one aspect, the mature polypeptide is amino acids 1 to 484 of SEQ ID NO: 17. Amino acids −31 to −1 of SEQ ID NO: 17 are a signal peptide. In one aspect, the mature polypeptide is amino acids 1 to 480 of SEQ ID NO: 20. Amino acids −30 to −1 of SEQ ID NO: 20 are a signal peptide. In one aspect, the mature polypeptide is amino acids 1 to 485 of SEQ ID NO: 23. Amino acids −31 to −1 of SEQ ID NO: 23 are a signal peptide. In one aspect, the mature polypeptide is amino acids 1 to 491 of SEQ ID NO: 26. Amino acids −28 to −1 of SEQ ID NO: 26 are a signal peptide. In one aspect, the mature polypeptide is amino acids 1 to 289 of SEQ ID NO: 29. Amino acids −19 to −1 of SEQ ID NO: 29 are a signal peptide. In one aspect, the mature polypeptide is amino acids 1 to 245 of SEQ ID NO: 32. Amino acids −15 to −1 of SEQ ID NO: 32 are a signal peptide. In one aspect, the mature polypeptide is amino acids 1 to 280 of SEQ ID NO: 35. Amino acids −19 to −1 of SEQ ID NO: 35 are a signal peptide. In one aspect, the mature polypeptide is amino acids 1 to 238 of SEQ ID NO: 38. Amino acids −22 to −1 of SEQ ID NO: 38 are a signal peptide. In one aspect, the mature polypeptide is amino acids 1 to 498 of SEQ ID NO: 41. Amino acids −23 to −1 of SEQ ID NO: 41 are a signal peptide. In one aspect, the mature polypeptide is amino acids 1 to 476 of SEQ ID NO: 44. Amino acids −25 to −1 of SEQ ID NO: 44 are a signal peptide. In one aspect, the mature polypeptide is amino acids 1 to 474 of SEQ ID NO: 47. Amino acids −28 to −1 of SEQ ID NO: 47 are a signal peptide. In one aspect, the mature polypeptide is amino acids 1 to 473 of SEQ ID NO: 50. Amino acids −29 to −1 of SEQ ID NO: 50 are a signal peptide. In one aspect, the mature polypeptide is amino acids 1 to 414 of SEQ ID NO: 53. Amino acids −25 to −1 of SEQ ID NO: 53 are a signal peptide. In one aspect, the mature polypeptide is amino acids 1 to 412 of SEQ ID NO: 56. Amino acids −31 to −1 of SEQ ID NO: 56 are a signal peptide. In one aspect, the mature polypeptide is amino acids 1 to 637 of SEQ ID NO: 59. Amino acids −35 to −1 of SEQ ID NO: 59 are a signal peptide. In one aspect, the mature polypeptide is amino acids 1 to 599 of SEQ ID NO: 62. Amino acids −33 to −1 of SEQ ID NO: 62 are a signal peptide. In one aspect, the mature polypeptide is amino acids 1 to 605 of SEQ ID NO: 65. Amino acids −31 to −1 of SEQ ID NO: 65 are a signal peptide. In one aspect, the mature polypeptide is amino acids 1 to 610 of SEQ ID NO: 68. Amino acids −36 to −1 of SEQ ID NO: 68 are a signal peptide. In one aspect, the mature polypeptide is amino acids 1 to 216 of SEQ ID NO: 71. Amino acids −20 to −1 of SEQ ID NO: 71 are a signal peptide. In one aspect, the mature polypeptide is amino acids 1 to 425 of SEQ ID NO: 74. Amino acids −26 to −1 of SEQ ID NO: 74 are a signal peptide. In one aspect, the mature polypeptide is amino acids 1 to 304 of SEQ ID NO: 77. Amino acids −25 to −1 of SEQ ID NO: 77 are a signal peptide. In one aspect, the mature polypeptide is amino acids 1 to 306 of SEQ ID NO: 80. Amino acids −29 to −1 of SEQ ID NO: 80 are a signal peptide. In one aspect, the mature polypeptide is amino acids 1 to 306 of SEQ ID NO: 83. Amino acids −23 to −1 of SEQ ID NO: 83 are a signal peptide. In one aspect, the mature polypeptide is amino acids 1 to 632 of SEQ ID NO: 86. Amino acids −35 to −1 of SEQ ID NO: 86 are a signal peptide. In one aspect, the mature polypeptide is amino acids 1 to 216 of SEQ ID NO: 89. Amino acids −24 to −1 of SEQ ID NO: 89 are a signal peptide. In one aspect, the mature polypeptide is amino acids 1 to 306 of SEQ ID NO: 92. Amino acids −27 to −1 of SEQ ID NO: 92 are a signal peptide. In one aspect, the mature polypeptide is amino acids 1 to 453 of SEQ ID NO: 95. Amino acids −29 to −1 of SEQ ID NO: 95 are a signal peptide. In one aspect, the mature polypeptide is amino acids 1 to 473 of SEQ ID NO: 98. Amino acids −29 to −1 of SEQ ID NO: 98 are a signal peptide. In one aspect, the mature polypeptide is amino acids 1 to 209 of SEQ ID NO: 101. Amino acids −27 to −1 of SEQ ID NO: 101 are a signal peptide. In one aspect, the mature polypeptide is amino acids 1 to 281 of SEQ ID NO: 104. Amino acids −24 to −1 of SEQ ID NO: 104 are a signal peptide. In one aspect, the mature polypeptide is amino acids 1 to 582 of SEQ ID NO: 107. Amino acids −57 to −1 of SEQ ID NO: 107 are a signal peptide.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

The term “mature polypeptide coding sequence” means a polynucleotide that encodes a mature polypeptide having peptidoglycan degrading activity.

In one aspect, the mature polypeptide coding sequence is nucleotides 88 to 1380 of SEQ ID NO: 1 and nucleotides 1 to 87 of SEQ ID NO: 1 encode a signal peptide. In one aspect, the mature polypeptide coding sequence is nucleotides 91 to 1545 of SEQ ID NO: 4 and nucleotides 1 to 90 of SEQ ID NO: 4 encode a signal peptide. In one aspect, the mature polypeptide coding sequence is nucleotides 79 to 1527 of SEQ ID NO: 7 and nucleotides 1 to 78 of SEQ ID NO: 7 encode a signal peptide. In one aspect, the mature polypeptide coding sequence is nucleotides 67 to 1479 of SEQ ID NO: 10 and nucleotides 1 to 66 of SEQ ID NO: 10 encode a signal peptide. In one aspect, the mature polypeptide coding sequence is nucleotides 1 to 1917 of SEQ ID NO: 13. In one aspect, the mature polypeptide coding sequence is nucleotides 94 to 1545 of SEQ ID NO: 16 and nucleotides 1 to 93 of SEQ ID NO: 16 encode a signal peptide. In one aspect, the mature polypeptide coding sequence is nucleotides 91 to 1530 of SEQ ID NO: 19 and nucleotides 1 to 90 of SEQ ID NO: 19 encode a signal peptide. In one aspect, the mature polypeptide coding sequence is nucleotides 94 to 1548 of SEQ ID NO: 22 and nucleotides 1 to 93 of SEQ ID NO: 22 encode a signal peptide. In one aspect, the mature polypeptide coding sequence is nucleotides 85 to 1557 of SEQ ID NO: 25 and nucleotides 1 to 84 of SEQ ID NO: 25 encode a signal peptide. In one aspect, the mature polypeptide coding sequence is nucleotides 58 to 924 of SEQ ID NO: 28 and nucleotides 1 to 57 of SEQ ID NO: 28 encode a signal peptide. In one aspect, the mature polypeptide coding sequence is nucleotides 46 to 780 of SEQ ID NO: 31 and nucleotides 1 to 45 of SEQ ID NO: 31 encode a signal peptide. In one aspect, the mature polypeptide coding sequence is nucleotides 58 to 897 of SEQ ID NO: 34 and nucleotides 1 to 57 of SEQ ID NO: 34 encode a signal peptide. In one aspect, the mature polypeptide coding sequence is nucleotides 67 to 780 of SEQ ID NO: 37 and nucleotides 1 to 66 of SEQ ID NO: 37 encode a signal peptide. In one aspect, the mature polypeptide coding sequence is nucleotides 70 to 1563 of SEQ ID NO: 40 and nucleotides 1 to 69 of SEQ ID NO: 40 encode a signal peptide. In one aspect, the mature polypeptide coding sequence is nucleotides 76 to 1503 of SEQ ID NO: 43 and nucleotides 1 to 75 of SEQ ID NO: 43 encode a signal peptide. In one aspect, the mature polypeptide coding sequence is nucleotides 85 to 1506 of SEQ ID NO: 46 and nucleotides 1 to 84 of SEQ ID NO: 46 encode a signal peptide. In one aspect, the mature polypeptide coding sequence is nucleotides 88 to 1506 of SEQ ID NO: 49 and nucleotides 1 to 87 of SEQ ID NO: 49 encode a signal peptide. In one aspect, the mature polypeptide coding sequence is nucleotides 76 to 1317 of SEQ ID NO: 52 and nucleotides 1 to 75 of SEQ ID NO: 52 encode a signal peptide. In one aspect, the mature polypeptide coding sequence is nucleotides 94 to 1329 of SEQ ID NO: 55 and nucleotides 1 to 93 of SEQ ID NO: 55 encode a signal peptide. In one aspect, the mature polypeptide coding sequence is nucleotides 106 to 2016 of SEQ ID NO: 58 and nucleotides 1 to 105 of SEQ ID NO: 58 encode a signal peptide. In one aspect, the mature polypeptide coding sequence is nucleotides 100 to 1896 of SEQ ID NO: 61 and nucleotides 1 to 99 of SEQ ID NO: 61 encode a signal peptide. In one aspect, the mature polypeptide coding sequence is nucleotides 94 to 1908 of SEQ ID NO: 64 and nucleotides 1 to 93 of SEQ ID NO: 64 encode a signal peptide. In one aspect, the mature polypeptide coding sequence is nucleotides 109 to 1938 of SEQ ID NO: 67 and nucleotides 1 to 108 of SEQ ID NO: 67 encode a signal peptide. In one aspect, the mature polypeptide coding sequence is nucleotides 61 to 708 of SEQ ID NO: 70 and nucleotides 1 to 60 of SEQ ID NO: 70 encode a signal peptide. In one aspect, the mature polypeptide coding sequence is nucleotides 79 to 1353 of SEQ ID NO: 73 and nucleotides 1 to 78 of SEQ ID NO: 73 encode a signal peptide. In one aspect, the mature polypeptide coding sequence is nucleotides 76 to 987 of SEQ ID NO: 76 and nucleotides 1 to 75 of SEQ ID NO: 76 encode a signal peptide. In one aspect, the mature polypeptide coding sequence is nucleotides 73 to 990 of SEQ ID NO: 79 and nucleotides 1 to 72 of SEQ ID NO: 79 encode a signal peptide. In one aspect, the mature polypeptide coding sequence is nucleotides 70 to 987 of SEQ ID NO: 82 and nucleotides 1 to 69 of SEQ ID NO: 82 encode a signal peptide. In one aspect, the mature polypeptide coding sequence is nucleotides 106 to 2001 of SEQ ID NO: 85 and nucleotides 1 to 105 of SEQ ID NO: 85 encode a signal peptide. In one aspect, the mature polypeptide coding sequence is nucleotides 73 to 720 of SEQ ID NO: 88 and nucleotides 1 to 72 of SEQ ID NO: 88 encode a signal peptide. In one aspect, the mature polypeptide coding sequence is nucleotides 82 to 999 of SEQ ID NO: 91 and nucleotides 1 to 81 of SEQ ID NO: 91 encode a signal peptide. In one aspect, the mature polypeptide coding sequence is nucleotides 88 to 1446 of SEQ ID NO: 94 and nucleotides 1 to 87 of SEQ ID NO: 94 encode a signal peptide. In one aspect, the mature polypeptide coding sequence is nucleotides 88 to 1506 of SEQ ID NO: 97 and nucleotides 1 to 87 of SEQ ID NO: 97 encode a signal peptide. In one aspect, the mature polypeptide coding sequence is nucleotides 82 to 708 of SEQ ID NO: 100 and nucleotides 1 to 81 of SEQ ID NO: 100 encode a signal peptide. In one aspect, the mature polypeptide coding sequence is nucleotides 73 to 915 of SEQ ID NO: 103 and nucleotides 1 to 72 of SEQ ID NO: 103 encode a signal peptide. In one aspect, the mature polypeptide coding sequence is nucleotides 172 to 1917 of SEQ ID NO: 106 and nucleotides 1 to 171 of SEQ ID NO: 106 encode a signal peptide.

The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

The term “operably linked” means a configuration in which a control sequence is placed at an appropriate position relative to the coding sequence of a polynucleotide such that the control sequence directs expression of the coding sequence.

The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

$$( Identical Residues \times 100 ) / ( Length of Alignment - Total Number of Gaps in Alignment )$$

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

$$( Identical Deoxyribonucleotides \times 100 ) / ( Length of Alignment - Total Number of Gaps in Alignment )$$

The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

The term “variant” means a polypeptide having peptidoglycan degrading activity comprising an alteration, i.e., a substitution, insertion, and/or deletion, at one or more positions. A substitution means replacement of the amino acid occupying a position with a different amino acid; a deletion means removal of the amino acid occupying a position; and an insertion means adding an amino acid adjacent to and immediately following the amino acid occupying a position.

Nomenclature: For purposes of the present invention, the nomenclature [E/Q] or [EQ] means that the amino acid at this position may be a glutamic acid (Glu, E) or a glutamine (Gln, Q). Likewise, the nomenclature [V/G/A/I] or [VGAI] means that the amino acid at this position may be a valine (Val, V), glycine (Gly, G), alanine (Ala, A) or isoleucine (Ile, I), and so forth for other combinations as described herein. Unless otherwise limited further, the amino acid X is defined such that it may be any of the 20 natural amino acids.

### DETAILED DESCRIPTION OF THE INVENTION

As mentioned in the background section above, textiles and surfaces such as laundry and dishes may become soiled with many different types of soiling. A single complex stain such as a food stain, sebum, dead cells debris, EPS or biofilm related stains is often composed of different organic material such as proteins, polysaccharides, grease etc., which are often difficult to remove completely with traditional detergent compositions. Further, such stains may give rise to disadvantages such as redeposition or malodor. The polypeptides of the invention address this problem, providing good cleaning effects on complex stains such as biofilm and EPS as well as reduced redeposition and malodor from, e.g., textiles and tableware. The polypeptides of the invention are peptidoglycan degrading enzymes having hydrolase activity and preferably N-acetylmuramyl-L-alanine amidase activity. The polypeptides of the invention comprise an amidase domain, preferably an Amidase_2 domain as defined in PFAM (PF01510, Pfam version 31.0; Finn, 2016, Nucleic Acids Research, Database Issue 44: D279-D285). Also, clusters or clades are described herein, defined by specific motifs shared by the polypeptides of the specific clades. A phylogenetic tree was constructed of polypeptide sequences containing an Amidase_2 domain. The phylogenetic tree was constructed from a multiple alignment of mature polypeptide sequences containing at least one Amidase_2 domain as described in Example 6.

One embodiment of the invention relates to a peptidoglycan degrading enzyme having hydrolase activity and preferably N-acetylmuramyl-L-alanine amidase activity. One embodiment of the invention relates to a peptidoglycan degrading enzyme having hydrolase activity and preferably N-acetylmuramyl-L-alanine amidase activity, wherein the polypeptide comprises the motif N[IV]X[AG][GAS]A[AY][LV]L (SEQ ID NO: 111), where X can be any naturally occurring amino acid, situated in positions corresponding to positions 85 to 93 in Micromonospora maritima (SEQ ID NO: 6).

The polypeptides containing an Amidase_2 domain can be separated into distinct sub-clusters. The sub-clusters are defined by one or more short sequence motifs, as well as containing an Amidase_2 domain as defined in PFAM (PF01510, Pfam version 31.0). We denoted one sub-cluster comprising the motif N[IV]X[AG][GAS]A[AY][LV]L (SEQ ID NO: 111) as the PGL clade. All polypeptide sequences containing an Amidase_2 domain as well as the motif will be denoted as belonging to the PGL clade.

In an embodiment, the present invention relates to polypeptides having a sequence identity to the mature polypeptide of SEQ ID NO: 2 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 2.

In a particular embodiment, the invention relates to polypeptides having a sequence identity to the mature polypeptide of SEQ ID NO: 2 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 2.

In an embodiment, the present invention relates to polypeptides having a sequence identity to the mature polypeptide of SEQ ID NO: 5 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 5.

In a particular embodiment, the invention relates to polypeptides having a sequence identity to the mature polypeptide of SEQ ID NO: 5 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 5.

In an embodiment, the present invention relates to polypeptides having a sequence identity to the mature polypeptide of SEQ ID NO: 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 8.

In a particular embodiment, the invention relates to polypeptides having a sequence identity to the mature polypeptide of SEQ ID NO: 8 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 8.

In an embodiment, the present invention relates to polypeptides having a sequence identity to the mature polypeptide of SEQ ID NO: 11 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 11.

In a particular embodiment, the invention relates to polypeptides having a sequence identity to the mature polypeptide of SEQ ID NO: 11 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 11.

In an embodiment, the present invention relates to polypeptides having a sequence identity to the mature polypeptide of SEQ ID NO: 14 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 14.

In a particular embodiment, the invention relates to polypeptides having a sequence identity to the mature polypeptide of SEQ ID NO: 14 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 14.

In an embodiment, the present invention relates to polypeptides having a sequence identity to the mature polypeptide of SEQ ID NO: 17 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 17.

In a particular embodiment, the invention relates to polypeptides having a sequence identity to the mature polypeptide of SEQ ID NO: 17 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 17.

In an embodiment, the present invention relates to polypeptides having a sequence identity to the mature polypeptide of SEQ ID NO: 20 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 20.

In a particular embodiment, the invention relates to polypeptides having a sequence identity to the mature polypeptide of SEQ ID NO: 20 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 20.

In an embodiment, the present invention relates to polypeptides having a sequence identity to the mature polypeptide of SEQ ID NO: 23 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 23.

In a particular embodiment, the invention relates to polypeptides having a sequence identity to the mature polypeptide of SEQ ID NO: 23 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 23.

In an embodiment, the present invention relates to polypeptides having a sequence identity to the mature polypeptide of SEQ ID NO: 26 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 26.

In a particular embodiment, the invention relates to polypeptides having a sequence identity to the mature polypeptide of SEQ ID NO: 26 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 26.

In an embodiment, the present invention relates to polypeptides having a sequence identity to the mature polypeptide of SEQ ID NO: 29 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 29.

In a particular embodiment, the invention relates to polypeptides having a sequence identity to the mature polypeptide of SEQ ID NO: 29 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 29.

In an embodiment, the present invention relates to polypeptides having a sequence identity to the mature polypeptide of SEQ ID NO: 32 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 32.

In a particular embodiment, the invention relates to polypeptides having a sequence identity to the mature polypeptide of SEQ ID NO: 32 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 32.

In an embodiment, the present invention relates to polypeptides having a sequence identity to the mature polypeptide of SEQ ID NO: 35 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 35.

In a particular embodiment, the invention relates to polypeptides having a sequence identity to the mature polypeptide of SEQ ID NO: 35 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 35.

In an embodiment, the present invention relates to polypeptides having a sequence identity to the mature polypeptide of SEQ ID NO: 38 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 38.

In a particular embodiment, the invention relates to polypeptides having a sequence identity to the mature polypeptide of SEQ ID NO: 38 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 38.

In an embodiment, the present invention relates to polypeptides having a sequence identity to the mature polypeptide of SEQ ID NO: 41 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 41.

In a particular embodiment, the invention relates to polypeptides having a sequence identity to the mature polypeptide of SEQ ID NO: 41 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 41.

In an embodiment, the present invention relates to polypeptides having a sequence identity to the mature polypeptide of SEQ ID NO: 44 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 44.

In a particular embodiment, the invention relates to polypeptides having a sequence identity to the mature polypeptide of SEQ ID NO: 44 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 44.

In an embodiment, the present invention relates to polypeptides having a sequence identity to the mature polypeptide of SEQ ID NO: 47 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 47.

In a particular embodiment, the invention relates to polypeptides having a sequence identity to the mature polypeptide of SEQ ID NO: 47 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 47.

In an embodiment, the present invention relates to polypeptides having a sequence identity to the mature polypeptide of SEQ ID NO: 50 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 50.

In a particular embodiment, the invention relates to polypeptides having a sequence identity to the mature polypeptide of SEQ ID NO: 50 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 50.

In an embodiment, the present invention relates to polypeptides having a sequence identity to the mature polypeptide of SEQ ID NO: 53 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 53.

In a particular embodiment, the invention relates to polypeptides having a sequence identity to the mature polypeptide of SEQ ID NO: 53 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 53.

In an embodiment, the present invention relates to polypeptides having a sequence identity to the mature polypeptide of SEQ ID NO: 56 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 56.

In a particular embodiment, the invention relates to polypeptides having a sequence identity to the mature polypeptide of SEQ ID NO: 56 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 56.

In an embodiment, the present invention relates to polypeptides having a sequence identity to the mature polypeptide of SEQ ID NO: 59 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 59.

In a particular embodiment, the invention relates to polypeptides having a sequence identity to the mature polypeptide of SEQ ID NO: 59 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 59.

In an embodiment, the present invention relates to polypeptides having a sequence identity to the mature polypeptide of SEQ ID NO: 62 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 62.

In a particular embodiment, the invention relates to polypeptides having a sequence identity to the mature polypeptide of SEQ ID NO: 62 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 62.

In an embodiment, the present invention relates to polypeptides having a sequence identity to the mature polypeptide of SEQ ID NO: 65 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 65.

In a particular embodiment, the invention relates to polypeptides having a sequence identity to the mature polypeptide of SEQ ID NO: 65 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 65.

In an embodiment, the present invention relates to polypeptides having a sequence identity to the mature polypeptide of SEQ ID NO: 68 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 68.

In a particular embodiment, the invention relates to polypeptides having a sequence identity to the mature polypeptide of SEQ ID NO: 68 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 68.

In an embodiment, the present invention relates to polypeptides having a sequence identity to the mature polypeptide of SEQ ID NO: 71 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 71.

In a particular embodiment, the invention relates to polypeptides having a sequence identity to the mature polypeptide of SEQ ID NO: 71 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 71.

In an embodiment, the present invention relates to polypeptides having a sequence identity to the mature polypeptide of SEQ ID NO: 74 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 74.

In a particular embodiment, the invention relates to polypeptides having a sequence identity to the mature polypeptide of SEQ ID NO: 74 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 74.

In an embodiment, the present invention relates to polypeptides having a sequence identity to the mature polypeptide of SEQ ID NO: 77 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 77.

In a particular embodiment, the invention relates to polypeptides having a sequence identity to the mature polypeptide of SEQ ID NO: 77 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 77.

In an embodiment, the present invention relates to polypeptides having a sequence identity to the mature polypeptide of SEQ ID NO: 80 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 80.

In a particular embodiment, the invention relates to polypeptides having a sequence identity to the mature polypeptide of SEQ ID NO: 80 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 80.

In an embodiment, the present invention relates to polypeptides having a sequence identity to the mature polypeptide of SEQ ID NO: 83 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 83.

In a particular embodiment, the invention relates to polypeptides having a sequence identity to the mature polypeptide of SEQ ID NO: 83 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 83.

In an embodiment, the present invention relates to polypeptides having a sequence identity to the mature polypeptide of SEQ ID NO: 86 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 86.

In a particular embodiment, the invention relates to polypeptides having a sequence identity to the mature polypeptide of SEQ ID NO: 86 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 86.

In an embodiment, the present invention relates to polypeptides having a sequence identity to the mature polypeptide of SEQ ID NO: 89 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 89.

In a particular embodiment, the invention relates to polypeptides having a sequence identity to the mature polypeptide of SEQ ID NO: 89 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 89.

In an embodiment, the present invention relates to polypeptides having a sequence identity to the mature polypeptide of SEQ ID NO: 92 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 92.

In a particular embodiment, the invention relates to polypeptides having a sequence identity to the mature polypeptide of SEQ ID NO: 92 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 92.

In an embodiment, the present invention relates to polypeptides having a sequence identity to the mature polypeptide of SEQ ID NO: 95 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 95.

In a particular embodiment, the invention relates to polypeptides having a sequence identity to the mature polypeptide of SEQ ID NO: 95 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 95.

In an embodiment, the present invention relates to polypeptides having a sequence identity to the mature polypeptide of SEQ ID NO: 98 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 98.

In a particular embodiment, the invention relates to polypeptides having a sequence identity to the mature polypeptide of SEQ ID NO: 98 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 98.

In an embodiment, the present invention relates to polypeptides having a sequence identity to the mature polypeptide of SEQ ID NO: 101 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 101.

In a particular embodiment, the invention relates to polypeptides having a sequence identity to the mature polypeptide of SEQ ID NO: 101 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 101.

In an embodiment, the present invention relates to polypeptides having a sequence identity to the mature polypeptide of SEQ ID NO: 104 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 104.

In a particular embodiment, the invention relates to polypeptides having a sequence identity to the mature polypeptide of SEQ ID NO: 104 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 104.

In an embodiment, the present invention relates to polypeptides having a sequence identity to the mature polypeptide of SEQ ID NO: 107 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have peptidoglycan degrading activity. In one aspect, the polypeptides differ by up to 10 amino acids, e.g., 1, 2, 3, 4, 5, 6, 7, 8, 9, or 10, from the mature polypeptide of SEQ ID NO: 107.

In a particular embodiment, the invention relates to polypeptides having a sequence identity to the mature polypeptide of SEQ ID NO: 107 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the peptidoglycan degrading activity of the mature polypeptide of SEQ ID NO: 107.

In a particular embodiment, the invention relates to polypeptides having a sequence identity to the polypeptide of SEQ ID NO: 3 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 6 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 9 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 12 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 15 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 18 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 21 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 24 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 27 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 30 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 33 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 36 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 39 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 42 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 45 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 48 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 51 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 54 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 57 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 60 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 63 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 66 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 69 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 72 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 75 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 78 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 81 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 84 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 87 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 90 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 93 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 96 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 99 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 102 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 105 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

In a particular embodiment, the invention relates to polypeptides having a sequence identity to the polypeptide of SEQ ID NO: 108 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has peptidoglycan degradation activity.

One embodiment of the invention relates to a polypeptide wherein the polypeptide comprises the motif N[IV]X[AG][GAS]A[AY][LV]L (SEQ ID NO: 111), wherein the polypeptide is selected from the group consisting of: SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 42, SEQ ID NO: 45, SEQ ID NO: 48, SEQ ID NO: 51, SEQ ID NO: 54, SEQ ID NO: 60, SEQ ID NO: 63, SEQ ID NO: 66, SEQ ID NO: 69, SEQ ID NO: 75, SEQ ID NO: 87, SEQ ID NO: 96, SEQ ID NO: 99 and SEQ ID NO: 108 and polypeptides having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity thereto, and wherein the polypeptide has peptidoglycan degradation activity.

One embodiment of the invention relates to a polypeptide wherein the polypeptide comprises the motif N[IV]X[AG][GAS]A[AY][LV]L (SEQ ID NO: 111), wherein the polypeptide is selected from the group consisting of: SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 42, SEQ ID NO: 45, SEQ ID NO: 48, SEQ ID NO: 51, SEQ ID NO: 54, SEQ ID NO: 60, SEQ ID NO: 63, SEQ ID NO: 66, SEQ ID NO: 69, SEQ ID NO: 75, SEQ ID NO: 87, SEQ ID NO: 96, SEQ ID NO: 99 and SEQ ID NO: 108 and polypeptides having at least 60% sequence identity thereto, and wherein the polypeptide has peptidoglycan degradation activity.

One embodiment of the invention relates to a polypeptide wherein the polypeptide comprises the motif N[IV]X[AG][GAS]A[AY][LV]L (SEQ ID NO: 111), wherein the polypeptide is selected from the group consisting of: SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 42, SEQ ID NO: 45, SEQ ID NO: 48, SEQ ID NO: 51, SEQ ID NO: 54, SEQ ID NO: 60, SEQ ID NO: 63, SEQ ID NO: 66, SEQ ID NO: 69, SEQ ID NO: 75, SEQ ID NO: 87, SEQ ID NO: 96, SEQ ID NO: 99 and SEQ ID NO: 108 and polypeptides having at least 70% sequence identity thereto, and wherein the polypeptide has peptidoglycan degradation activity.

One embodiment of the invention relates to a polypeptide wherein the polypeptide comprises the motif N[IV]X[AG][GAS]A[AY][LV]L (SEQ ID NO: 111), wherein the polypeptide is selected from the group consisting of: SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 42, SEQ ID NO: 45, SEQ ID NO: 48, SEQ ID NO: 51, SEQ ID NO: 54, SEQ ID NO: 60, SEQ ID NO: 63, SEQ ID NO: 66, SEQ ID NO: 69, SEQ ID NO: 75, SEQ ID NO: 87, SEQ ID NO: 96, SEQ ID NO: 99 and SEQ ID NO: 108 and polypeptides having at least 80% sequence identity thereto, and wherein the polypeptide has peptidoglycan degradation activity.

One embodiment of the invention relates to a polypeptide wherein the polypeptide comprises the motif N[IV]X[AG][GAS]A[AY][LV]L (SEQ ID NO: 111), wherein the polypeptide is selected from the group consisting of: SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 42, SEQ ID NO: 45, SEQ ID NO: 48, SEQ ID NO: 51, SEQ ID NO: 54, SEQ ID NO: 60, SEQ ID NO: 63, SEQ ID NO: 66, SEQ ID NO: 69, SEQ ID NO: 75, SEQ ID NO: 87, SEQ ID NO: 96, SEQ ID NO: 99 and SEQ ID NO: 108 and polypeptides having at least 90% sequence identity thereto, and wherein the polypeptide has peptidoglycan degradation activity.

One embodiment of the invention relates to a polypeptide wherein the polypeptide comprises the motif N[IV]X[AG][GAS]A[AY][LV]L (SEQ ID NO: 111), wherein the polypeptide is selected from the group consisting of: SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 42, SEQ ID NO: 45, SEQ ID NO: 48, SEQ ID NO: 51, SEQ ID NO: 54, SEQ ID NO: 60, SEQ ID NO: 63, SEQ ID NO: 66, SEQ ID NO: 69, SEQ ID NO: 75, SEQ ID NO: 87, SEQ ID NO: 96, SEQ ID NO: 99 and SEQ ID NO: 108 and polypeptides having at least 95% sequence identity thereto, and wherein the polypeptide has peptidoglycan degradation activity.

One embodiment of the invention relates to a polypeptide wherein the polypeptide comprises the motif N[IV]X[AG][GAS]A[AY][LV]L (SEQ ID NO: 111), wherein the polypeptide is selected from the group consisting of: SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 42, SEQ ID NO: 45, SEQ ID NO: 48, SEQ ID NO: 51, SEQ ID NO: 54, SEQ ID NO: 60, SEQ ID NO: 63, SEQ ID NO: 66, SEQ ID NO: 69, SEQ ID NO: 75, SEQ ID NO: 87, SEQ ID NO: 96, SEQ ID NO: 99 and SEQ ID NO: 108 and polypeptides having at least 98% sequence identity thereto, and wherein the polypeptide has peptidoglycan degradation activity.

One embodiment of the invention relates to a polypeptide wherein the polypeptide comprises the motif N[IV]X[AG][GAS]A[AY][LV]L (SEQ ID NO: 111), wherein the polypeptide is selected from the group consisting of: SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 42, SEQ ID NO: 45, SEQ ID NO: 48, SEQ ID NO: 51, SEQ ID NO: 54, SEQ ID NO: 60, SEQ ID NO: 63, SEQ ID NO: 66, SEQ ID NO: 69, SEQ ID NO: 75, SEQ ID NO: 87, SEQ ID NO: 96, SEQ ID NO: 99 and SEQ ID NO: 108 and polypeptides having at least 99% sequence identity thereto, and wherein the polypeptide has peptidoglycan degradation activity.

In any of the embodiments disclosed herein, the polypeptide has preferably been isolated, i.e., the polypeptide is in an “isolated” form or environment as defined above.

One polypeptide of the present invention preferably comprises or consists of the amino acid sequence of SEQ ID NO: 2 or an allelic variant thereof; or is a fragment thereof having peptidoglycan degradation activity. In another aspect, the polypeptide comprises or consists of the mature polypeptide shown in SEQ ID NO: 3. In another aspect, the polypeptide comprises or consists of amino acids 1 to 431 of SEQ ID NO: 2.

A polypeptide of the present invention preferably comprises or consists of the amino acid sequence of SEQ ID NO: 5 or an allelic variant thereof; or is a fragment thereof having peptidoglycan degradation activity. In another aspect, the polypeptide comprises or consists of the mature polypeptide shown in SEQ ID NO: 6. In another aspect, the polypeptide comprises or consists of amino acids 1 to 485 of SEQ ID NO: 5.

A polypeptide of the present invention preferably comprises or consists of the amino acid sequence of SEQ ID NO: 8 or an allelic variant thereof; or is a fragment thereof having peptidoglycan degradation activity. In another aspect, the polypeptide comprises or consists of the mature polypeptide shown in SEQ ID NO: 9. In another aspect, the polypeptide comprises or consists of amino acids 1 to 483 of SEQ ID NO: 8.

A polypeptide of the present invention preferably comprises or consists of the amino acid sequence of SEQ ID NO: 11 or an allelic variant thereof; or is a fragment thereof having peptidoglycan degradation activity. In another aspect, the polypeptide comprises or consists of the mature polypeptide shown in SEQ ID NO: 12. In another aspect, the polypeptide comprises or consists of amino acids 1 to 471 of SEQ ID NO: 11.

A polypeptide of the present invention preferably comprises or consists of the amino acid sequence of SEQ ID NO: 14 or an allelic variant thereof; or is a fragment thereof having peptidoglycan degradation activity. In another aspect, the polypeptide comprises or consists of the mature polypeptide shown in SEQ ID NO: 15. In another aspect, the polypeptide comprises or consists of amino acids 1 to 639 of SEQ ID NO: 14.

A polypeptide of the present invention preferably comprises or consists of the amino acid sequence of SEQ ID NO: 17 or an allelic variant thereof; or is a fragment thereof having peptidoglycan degradation activity. In another aspect, the polypeptide comprises or consists of the mature polypeptide shown in SEQ ID NO: 18. In another aspect, the polypeptide comprises or consists of amino acids 1 to 484 of SEQ ID NO: 17.

A polypeptide of the present invention preferably comprises or consists of the amino acid sequence of SEQ ID NO: 20 or an allelic variant thereof; or is a fragment thereof having peptidoglycan degradation activity. In another aspect, the polypeptide comprises or consists of the mature polypeptide shown in SEQ ID NO: 21. In another aspect, the polypeptide comprises or consists of amino acids 1 to 480 of SEQ ID NO: 20.

A polypeptide of the present invention preferably comprises or consists of the amino acid sequence of SEQ ID NO: 23 or an allelic variant thereof; or is a fragment thereof having peptidoglycan degradation activity. In another aspect, the polypeptide comprises or consists of the mature polypeptide shown in SEQ ID NO: 24. In another aspect, the polypeptide comprises or consists of amino acids 1 to 485 of SEQ ID NO: 23.

A polypeptide of the present invention preferably comprises or consists of the amino acid sequence of SEQ ID NO: 26 or an allelic variant thereof; or is a fragment thereof having peptidoglycan degradation activity. In another aspect, the polypeptide comprises or consists of the mature polypeptide shown in SEQ ID NO: 27. In another aspect, the polypeptide comprises or consists of amino acids 1 to 491 of SEQ ID NO: 26.

A polypeptide of the present invention preferably comprises or consists of the amino acid sequence of SEQ ID NO: 29 or an allelic variant thereof; or is a fragment thereof having peptidoglycan degradation activity. In another aspect, the polypeptide comprises or consists of the mature polypeptide shown in SEQ ID NO: 30. In another aspect, the polypeptide comprises or consists of amino acids 1 to 289 of SEQ ID NO: 29.

A polypeptide of the present invention preferably comprises or consists of the amino acid sequence of SEQ ID NO: 32 or an allelic variant thereof; or is a fragment thereof having peptidoglycan degradation activity. In another aspect, the polypeptide comprises or consists of the mature polypeptide shown in SEQ ID NO: 33. In another aspect, the polypeptide comprises or consists of amino acids 1 to 245 of SEQ ID NO: 32.

A polypeptide of the present invention preferably comprises or consists of the amino acid sequence of SEQ ID NO: 35 or an allelic variant thereof; or is a fragment thereof having peptidoglycan degradation activity. In another aspect, the polypeptide comprises or consists of the mature polypeptide shown in SEQ ID NO: 36. In another aspect, the polypeptide comprises or consists of amino acids 1 to 280 of SEQ ID NO: 35.

A polypeptide of the present invention preferably comprises or consists of the amino acid sequence of SEQ ID NO: 38 or an allelic variant thereof; or is a fragment thereof having peptidoglycan degradation activity. In another aspect, the polypeptide comprises or consists of the mature polypeptide shown in SEQ ID NO: 39. In another aspect, the polypeptide comprises or consists of amino acids 1 to 238 of SEQ ID NO: 38.

A polypeptide of the present invention preferably comprises or consists of the amino acid sequence of SEQ ID NO: 41 or an allelic variant thereof; or is a fragment thereof having peptidoglycan degradation activity. In another aspect, the polypeptide comprises or consists of the mature polypeptide shown in SEQ ID NO: 42. In another aspect, the polypeptide comprises or consists of amino acids 1 to 498 of SEQ ID NO: 41.

A polypeptide of the present invention preferably comprises or consists of the amino acid sequence of SEQ ID NO: 44 or an allelic variant thereof; or is a fragment thereof having peptidoglycan degradation activity. In another aspect, the polypeptide comprises or consists of the mature polypeptide shown in SEQ ID NO: 45. In another aspect, the polypeptide comprises or consists of amino acids 1 to 476 of SEQ ID NO: 44.

A polypeptide of the present invention preferably comprises or consists of the amino acid sequence of SEQ ID NO: 47 or an allelic variant thereof; or is a fragment thereof having peptidoglycan degradation activity. In another aspect, the polypeptide comprises or consists of the mature polypeptide shown in SEQ ID NO: 48. In another aspect, the polypeptide comprises or consists of amino acids 1 to 474 of SEQ ID NO: 47.

A polypeptide of the present invention preferably comprises or consists of the amino acid sequence of SEQ ID NO: 50 or an allelic variant thereof; or is a fragment thereof having peptidoglycan degradation activity. In another aspect, the polypeptide comprises or consists of the mature polypeptide shown in SEQ ID NO: 51. In another aspect, the polypeptide comprises or consists of amino acids 1 to 473 of SEQ ID NO: 50.

A polypeptide of the present invention preferably comprises or consists of the amino acid sequence of SEQ ID NO: 53 or an allelic variant thereof; or is a fragment thereof having peptidoglycan degradation activity. In another aspect, the polypeptide comprises or consists of the mature polypeptide shown in SEQ ID NO: 54. In another aspect, the polypeptide comprises or consists of amino acids 1 to 414 of SEQ ID NO: 53.

A polypeptide of the present invention preferably comprises or consists of the amino acid sequence of SEQ ID NO: 56 or an allelic variant thereof; or is a fragment thereof having peptidoglycan degradation activity. In another aspect, the polypeptide comprises or consists of the mature polypeptide shown in SEQ ID NO: 57. In another aspect, the polypeptide comprises or consists of amino acids 1 to 412 of SEQ ID NO: 56.

A polypeptide of the present invention preferably comprises or consists of the amino acid sequence of SEQ ID NO: 59 or an allelic variant thereof; or is a fragment thereof having peptidoglycan degradation activity. In another aspect, the polypeptide comprises or consists of the mature polypeptide shown in SEQ ID NO: 60. In another aspect, the polypeptide comprises or consists of amino acids 1 to 637 of SEQ ID NO: 59.

A polypeptide of the present invention preferably comprises or consists of the amino acid sequence of SEQ ID NO: 62 or an allelic variant thereof; or is a fragment thereof having peptidoglycan degradation activity. In another aspect, the polypeptide comprises or consists of the mature polypeptide shown in SEQ ID NO: 63. In another aspect, the polypeptide comprises or consists of amino acids 1 to 599 of SEQ ID NO: 62.

A polypeptide of the present invention preferably comprises or consists of the amino acid sequence of SEQ ID NO: 65 or an allelic variant thereof; or is a fragment thereof having peptidoglycan degradation activity. In another aspect, the polypeptide comprises or consists of the mature polypeptide shown in SEQ ID NO: 66. In another aspect, the polypeptide comprises or consists of amino acids 1 to 605 of SEQ ID NO: 65.

A polypeptide of the present invention preferably comprises or consists of the amino acid sequence of SEQ ID NO: 68 or an allelic variant thereof; or is a fragment thereof having peptidoglycan degradation activity. In another aspect, the polypeptide comprises or consists of the mature polypeptide shown in SEQ ID NO: 69. In another aspect, the polypeptide comprises or consists of amino acids 1 to 610 of SEQ ID NO: 68.

A polypeptide of the present invention preferably comprises or consists of the amino acid sequence of SEQ ID NO: 71 or an allelic variant thereof; or is a fragment thereof having peptidoglycan degradation activity. In another aspect, the polypeptide comprises or consists of the mature polypeptide shown in SEQ ID NO: 72. In another aspect, the polypeptide comprises or consists of amino acids 1 to 216 of SEQ ID NO: 71.

A polypeptide of the present invention preferably comprises or consists of the amino acid sequence of SEQ ID NO: 74 or an allelic variant thereof; or is a fragment thereof having peptidoglycan degradation activity. In another aspect, the polypeptide comprises or consists of the mature polypeptide shown in SEQ ID NO: 75. In another aspect, the polypeptide comprises or consists of amino acids 1 to 425 of SEQ ID NO: 74.

A polypeptide of the present invention preferably comprises or consists of the amino acid sequence of SEQ ID NO: 77 or an allelic variant thereof; or is a fragment thereof having peptidoglycan degradation activity. In another aspect, the polypeptide comprises or consists of the mature polypeptide shown in SEQ ID NO: 78. In another aspect, the polypeptide comprises or consists of amino acids 1 to 304 of SEQ ID NO: 77.

A polypeptide of the present invention preferably comprises or consists of the amino acid sequence of SEQ ID NO: 80 or an allelic variant thereof; or is a fragment thereof having peptidoglycan degradation activity. In another aspect, the polypeptide comprises or consists of the mature polypeptide shown in SEQ ID NO: 81. In another aspect, the polypeptide comprises or consists of amino acids 1 to 306 of SEQ ID NO: 80.

A polypeptide of the present invention preferably comprises or consists of the amino acid sequence of SEQ ID NO: 83 or an allelic variant thereof; or is a fragment thereof having peptidoglycan degradation activity. In another aspect, the polypeptide comprises or consists of the mature polypeptide shown in SEQ ID NO: 84. In another aspect, the polypeptide comprises or consists of amino acids 1 to 306 of SEQ ID NO: 83.

A polypeptide of the present invention preferably comprises or consists of the amino acid sequence of SEQ ID NO: 86 or an allelic variant thereof; or is a fragment thereof having peptidoglycan degradation activity. In another aspect, the polypeptide comprises or consists of the mature polypeptide shown in SEQ ID NO: 87. In another aspect, the polypeptide comprises or consists of amino acids 1 to 632 of SEQ ID NO: 86.

A polypeptide of the present invention preferably comprises or consists of the amino acid sequence of SEQ ID NO: 89 or an allelic variant thereof; or is a fragment thereof having peptidoglycan degradation activity. In another aspect, the polypeptide comprises or consists of the mature polypeptide shown in SEQ ID NO: 90. In another aspect, the polypeptide comprises or consists of amino acids 1 to 216 of SEQ ID NO: 89.

A polypeptide of the present invention preferably comprises or consists of the amino acid sequence of SEQ ID NO: 92 or an allelic variant thereof; or is a fragment thereof having peptidoglycan degradation activity. In another aspect, the polypeptide comprises or consists of the mature polypeptide shown in SEQ ID NO: 93. In another aspect, the polypeptide comprises or consists of amino acids 1 to 306 of SEQ ID NO: 92.

A polypeptide of the present invention preferably comprises or consists of the amino acid sequence of SEQ ID NO: 95 or an allelic variant thereof; or is a fragment thereof having peptidoglycan degradation activity. In another aspect, the polypeptide comprises or consists of the mature polypeptide shown in SEQ ID NO: 96. In another aspect, the polypeptide comprises or consists of amino acids 1 to 453 of SEQ ID NO: 95.

A polypeptide of the present invention preferably comprises or consists of the amino acid sequence of SEQ ID NO: 98 or an allelic variant thereof; or is a fragment thereof having peptidoglycan degradation activity. In another aspect, the polypeptide comprises or consists of the mature polypeptide shown in SEQ ID NO: 99. In another aspect, the polypeptide comprises or consists of amino acids 1 to 473 of SEQ ID NO: 98.

A polypeptide of the present invention preferably comprises or consists of the amino acid sequence of SEQ ID NO: 101 or an allelic variant thereof; or is a fragment thereof having peptidoglycan degradation activity. In another aspect, the polypeptide comprises or consists of the mature polypeptide shown in SEQ ID NO: 102. In another aspect, the polypeptide comprises or consists of amino acids 1 to 209 of SEQ ID NO: 101.

A polypeptide of the present invention preferably comprises or consists of the amino acid sequence of SEQ ID NO: 104 or an allelic variant thereof; or is a fragment thereof having peptidoglycan degradation activity. In another aspect, the polypeptide comprises or consists of the mature polypeptide shown in SEQ ID NO: 105. In another aspect, the polypeptide comprises or consists of amino acids 1 to 281 of SEQ ID NO: 104.

A polypeptide of the present invention preferably comprises or consists of the amino acid sequence of SEQ ID NO: 107 or an allelic variant thereof; or is a fragment thereof having peptidoglycan degradation activity. In another aspect, the polypeptide comprises or consists of the mature polypeptide shown in SEQ ID NO: 108. In another aspect, the polypeptide comprises or consists of amino acids 1 to 582 of SEQ ID NO: 107.

In another embodiment, the present invention relates to a polypeptide having peptidoglycan degradation activity encoded by a polynucleotide that hybridizes under very low stringency conditions, low stringency conditions, medium stringency conditions, medium-high stringency conditions, high stringency conditions, or very high stringency conditions with (i) SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103 or SEQ ID NO: 106, (ii) the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103 or SEQ ID NO: 106, or (iii) the full-length complement of (i) or (ii) (Sambrook et al., 1989, Molecular Cloning, A Laboratory Manual, 2d edition, Cold Spring Harbor, New York). Such polypeptides have preferably been isolated.

The polynucleotide of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106 or a subsequence thereof, as well as a fragment thereof may be used to design nucleic acid probes to identify and clone DNA encoding polypeptides having peptidoglycan degradation activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having peptidoglycan degradation activity. Genomic or other DNA from such other strains may be separated by agarose or polyacrylamide gel electrophoresis, or other separation techniques. DNA from the libraries or the separated DNA may be transferred to and immobilized on nitrocellulose or another suitable carrier material. In order to identify a clone or DNA that hybridizes with SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106 or a subsequence thereof, the carrier material is used in a Southern blot.

For purposes of the present invention, hybridization indicates that the polynucleotide hybridizes to a labeled nucleic acid probe corresponding to (i) SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103 or SEQ ID NO: 106; (ii) the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103 or SEQ ID NO: 106; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In another embodiment, the present invention relates to a polypeptide having peptidoglycan degradation activity encoded by a polynucleotide having a sequence identity to the mature polypeptide coding sequence of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 4 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 7 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 10 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 13 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 16 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 19 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 22 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 25 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 28 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 31 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 34 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 37 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 40 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 43 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 46 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 49 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 52 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 55 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 58 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 61 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 64 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 67 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 70 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 73 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 76 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 79 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 82 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 85 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 88 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 91 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 94 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 97 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 100 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 103 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having peptidoglycan degradation activity encoded by a polynucleotide having a sequence identity to the mature polypeptide coding sequence of SEQ ID NO: 106 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to variants of the polypeptide shown in SEQ ID NO: 3 comprising a substitution, deletion, and/or insertion at one or more positions. In an embodiment, the number of amino acid substitutions, deletions and/or insertions introduced into the polypeptide shown SEQ ID NO: 3 is up to 10, e.g., 1, 2, 3, 4, 5, 6, 7, 8, 9, or 10.

In another embodiment, the present invention relates to variants of the polypeptide shown in SEQ ID NO: 6 comprising a substitution, deletion, and/or insertion at one or more positions. In an embodiment, the number of amino acid substitutions, deletions and/or insertions introduced into the polypeptide shown SEQ ID NO: 6 is up to 10, e.g., 1, 2, 3, 4, 5, 6, 7, 8, 9, or 10.

In another embodiment, the present invention relates to variants of the polypeptide shown in SEQ ID NO: 9 comprising a substitution, deletion, and/or insertion at one or more positions. In an embodiment, the number of amino acid substitutions, deletions and/or insertions introduced into the polypeptide shown SEQ ID NO: 9 is up to 10, e.g., 1, 2, 3, 4, 5, 6, 7, 8, 9, or 10.

In another embodiment, the present invention relates to variants of the polypeptide shown in SEQ ID NO: 12 comprising a substitution, deletion, and/or insertion at one or more positions. In an embodiment, the number of amino acid substitutions, deletions and/or insertions introduced into the polypeptide shown SEQ ID NO: 12 is up to 10, e.g., 1, 2, 3, 4, 5, 6, 7, 8, 9, or 10.

In another embodiment, the present invention relates to variants of the polypeptide shown in SEQ ID NO: 15 comprising a substitution, deletion, and/or insertion at one or more positions. In an embodiment, the number of amino acid substitutions, deletions and/or insertions introduced into the polypeptide shown SEQ ID NO: 15 is up to 10, e.g., 1, 2, 3, 4, 5, 6, 7, 8, 9, or 10.

In another embodiment, the present invention relates to variants of the polypeptide shown in SEQ ID NO: 18 comprising a substitution, deletion, and/or insertion at one or more positions. In an embodiment, the number of amino acid substitutions, deletions and/or insertions introduced into the polypeptide shown SEQ ID NO: 18 is up to 10, e.g., 1, 2, 3, 4, 5, 6, 7, 8, 9, or 10.

In another embodiment, the present invention relates to variants of the polypeptide shown in SEQ ID NO: 21 comprising a substitution, deletion, and/or insertion at one or more positions. In an embodiment, the number of amino acid substitutions, deletions and/or insertions introduced into the polypeptide shown SEQ ID NO: 21 is up to 10, e.g., 1, 2, 3, 4, 5, 6, 7, 8, 9, or 10.

In another embodiment, the present invention relates to variants of the polypeptide shown in SEQ ID NO: 24 comprising a substitution, deletion, and/or insertion at one or more positions. In an embodiment, the number of amino acid substitutions, deletions and/or insertions introduced into the polypeptide shown SEQ ID NO: 24 is up to 10, e.g., 1, 2, 3, 4, 5, 6, 7, 8, 9, or 10.

In another embodiment, the present invention relates to variants of the polypeptide shown in SEQ ID NO: 27 comprising a substitution, deletion, and/or insertion at one or more positions. In an embodiment, the number of amino acid substitutions, deletions and/or insertions introduced into the polypeptide shown SEQ ID NO: 27 is up to 10, e.g., 1, 2, 3, 4, 5, 6, 7, 8, 9, or 10.

In another embodiment, the present invention relates to variants of the polypeptide shown in SEQ ID NO: 30 comprising a substitution, deletion, and/or insertion at one or more positions. In an embodiment, the number of amino acid substitutions, deletions and/or insertions introduced into the polypeptide shown SEQ ID NO: 30 is up to 10, e.g., 1, 2, 3, 4, 5, 6, 7, 8, 9, or 10.

In another embodiment, the present invention relates to variants of the polypeptide shown in SEQ ID NO: 33 comprising a substitution, deletion, and/or insertion at one or more positions. In an embodiment, the number of amino acid substitutions, deletions and/or insertions introduced into the polypeptide shown SEQ ID NO: 33 is up to 10, e.g., 1, 2, 3, 4, 5, 6, 7, 8, 9, or 10.

In another embodiment, the present invention relates to variants of the polypeptide shown in SEQ ID NO: 36 comprising a substitution, deletion, and/or insertion at one or more positions. In an embodiment, the number of amino acid substitutions, deletions and/or insertions introduced into the polypeptide shown SEQ ID NO: 36 is up to 10, e.g., 1, 2, 3, 4, 5, 6, 7, 8, 9, or 10.

In another embodiment, the present invention relates to variants of the polypeptide shown in SEQ ID NO: 39 comprising a substitution, deletion, and/or insertion at one or more positions. In an embodiment, the number of amino acid substitutions, deletions and/or insertions introduced into the polypeptide shown SEQ ID NO: 39 is up to 10, e.g., 1, 2, 3, 4, 5, 6, 7, 8, 9, or 10.

In another embodiment, the present invention relates to variants of the polypeptide shown in SEQ ID NO: 42 comprising a substitution, deletion, and/or insertion at one or more positions. In an embodiment, the number of amino acid substitutions, deletions and/or insertions introduced into the polypeptide shown SEQ ID NO: 42 is up to 10, e.g., 1, 2, 3, 4, 5, 6, 7, 8, 9, or 10.

In another embodiment, the present invention relates to variants of the polypeptide shown in SEQ ID NO: 45 comprising a substitution, deletion, and/or insertion at one or more positions. In an embodiment, the number of amino acid substitutions, deletions and/or insertions introduced into the polypeptide shown SEQ ID NO: 45 is up to 10, e.g., 1, 2, 3, 4, 5, 6, 7, 8, 9, or 10.

In another embodiment, the present invention relates to variants of the polypeptide shown in SEQ ID NO: 48 comprising a substitution, deletion, and/or insertion at one or more positions. In an embodiment, the number of amino acid substitutions, deletions and/or insertions introduced into the polypeptide shown SEQ ID NO: 48 is up to 10, e.g., 1, 2, 3, 4, 5, 6, 7, 8, 9, or 10.

In another embodiment, the present invention relates to variants of the polypeptide shown in SEQ ID NO: 51 comprising a substitution, deletion, and/or insertion at one or more positions. In an embodiment, the number of amino acid substitutions, deletions and/or insertions introduced into the polypeptide shown SEQ ID NO: 51 is up to 10, e.g., 1, 2, 3, 4, 5, 6, 7, 8, 9, or 10.

In another embodiment, the present invention relates to variants of the polypeptide shown in SEQ ID NO: 54 comprising a substitution, deletion, and/or insertion at one or more positions. In an embodiment, the number of amino acid substitutions, deletions and/or insertions introduced into the polypeptide shown SEQ ID NO: 54 is up to 10, e.g., 1, 2, 3, 4, 5, 6, 7, 8, 9, or 10.

In another embodiment, the present invention relates to variants of the polypeptide shown in SEQ ID NO: 57 comprising a substitution, deletion, and/or insertion at one or more positions. In an embodiment, the number of amino acid substitutions, deletions and/or insertions introduced into the polypeptide shown SEQ ID NO: 57 is up to 10, e.g., 1, 2, 3, 4, 5, 6, 7, 8, 9, or 10.

In another embodiment, the present invention relates to variants of the polypeptide shown in SEQ ID NO: 60 comprising a substitution, deletion, and/or insertion at one or more positions. In an embodiment, the number of amino acid substitutions, deletions and/or insertions introduced into the polypeptide shown SEQ ID NO: 60 is up to 10, e.g., 1, 2, 3, 4, 5, 6, 7, 8, 9, or 10.

In another embodiment, the present invention relates to variants of the polypeptide shown in SEQ ID NO: 63 comprising a substitution, deletion, and/or insertion at one or more positions. In an embodiment, the number of amino acid substitutions, deletions and/or insertions introduced into the polypeptide shown SEQ ID NO: 63 is up to 10, e.g., 1, 2, 3, 4, 5, 6, 7, 8, 9, or 10.

In another embodiment, the present invention relates to variants of the polypeptide shown in SEQ ID NO: 66 comprising a substitution, deletion, and/or insertion at one or more positions. In an embodiment, the number of amino acid substitutions, deletions and/or insertions introduced into the polypeptide shown SEQ ID NO: 66 is up to 10, e.g., 1, 2, 3, 4, 5, 6, 7, 8, 9, or 10.

In another embodiment, the present invention relates to variants of the polypeptide shown in SEQ ID NO: 69 comprising a substitution, deletion, and/or insertion at one or more positions. In an embodiment, the number of amino acid substitutions, deletions and/or insertions introduced into the polypeptide shown SEQ ID NO: 69 is up to 10, e.g., 1, 2, 3, 4, 5, 6, 7, 8, 9, or 10.

In another embodiment, the present invention relates to variants of the polypeptide shown in SEQ ID NO: 72 comprising a substitution, deletion, and/or insertion at one or more positions. In an embodiment, the number of amino acid substitutions, deletions and/or insertions introduced into the polypeptide shown SEQ ID NO: 72 is up to 10, e.g., 1, 2, 3, 4, 5, 6, 7, 8, 9, or 10.

In another embodiment, the present invention relates to variants of the polypeptide shown in SEQ ID NO: 75 comprising a substitution, deletion, and/or insertion at one or more positions. In an embodiment, the number of amino acid substitutions, deletions and/or insertions introduced into the polypeptide shown SEQ ID NO: 75 is up to 10, e.g., 1, 2, 3, 4, 5, 6, 7, 8, 9, or 10.

In another embodiment, the present invention relates to variants of the polypeptide shown in SEQ ID NO: 78 comprising a substitution, deletion, and/or insertion at one or more positions. In an embodiment, the number of amino acid substitutions, deletions and/or insertions introduced into the polypeptide shown SEQ ID NO: 78 is up to 10, e.g., 1, 2, 3, 4, 5, 6, 7, 8, 9, or 10.

In another embodiment, the present invention relates to variants of the polypeptide shown in SEQ ID NO: 81 comprising a substitution, deletion, and/or insertion at one or more positions. In an embodiment, the number of amino acid substitutions, deletions and/or insertions introduced into the polypeptide shown SEQ ID NO: 81 is up to 10, e.g., 1, 2, 3, 4, 5, 6, 7, 8, 9, or 10.

In another embodiment, the present invention relates to variants of the polypeptide shown in SEQ ID NO: 84 comprising a substitution, deletion, and/or insertion at one or more positions. In an embodiment, the number of amino acid substitutions, deletions and/or insertions introduced into the polypeptide shown SEQ ID NO: 84 is up to 10, e.g., 1, 2, 3, 4, 5, 6, 7, 8, 9, or 10.

In another embodiment, the present invention relates to variants of the polypeptide shown in SEQ ID NO: 87 comprising a substitution, deletion, and/or insertion at one or more positions. In an embodiment, the number of amino acid substitutions, deletions and/or insertions introduced into the polypeptide shown SEQ ID NO: 87 is up to 10, e.g., 1, 2, 3, 4, 5, 6, 7, 8, 9, or 10.

In another embodiment, the present invention relates to variants of the polypeptide shown in SEQ ID NO: 90 comprising a substitution, deletion, and/or insertion at one or more positions. In an embodiment, the number of amino acid substitutions, deletions and/or insertions introduced into the polypeptide shown SEQ ID NO: 90 is up to 10, e.g., 1, 2, 3, 4, 5, 6, 7, 8, 9, or 10.

In another embodiment, the present invention relates to variants of the polypeptide shown in SEQ ID NO: 93 comprising a substitution, deletion, and/or insertion at one or more positions. In an embodiment, the number of amino acid substitutions, deletions and/or insertions introduced into the polypeptide shown SEQ ID NO: 93 is up to 10, e.g., 1, 2, 3, 4, 5, 6, 7, 8, 9, or 10.

In another embodiment, the present invention relates to variants of the polypeptide shown in SEQ ID NO: 96 comprising a substitution, deletion, and/or insertion at one or more positions. In an embodiment, the number of amino acid substitutions, deletions and/or insertions introduced into the polypeptide shown SEQ ID NO: 96 is up to 10, e.g., 1, 2, 3, 4, 5, 6, 7, 8, 9, or 10.

In another embodiment, the present invention relates to variants of the polypeptide shown in SEQ ID NO: 99 comprising a substitution, deletion, and/or insertion at one or more positions. In an embodiment, the number of amino acid substitutions, deletions and/or insertions introduced into the polypeptide shown SEQ ID NO: 99 is up to 10, e.g., 1, 2, 3, 4, 5, 6, 7, 8, 9, or 10.

In another embodiment, the present invention relates to variants of the polypeptide shown in SEQ ID NO: 102 comprising a substitution, deletion, and/or insertion at one or more positions. In an embodiment, the number of amino acid substitutions, deletions and/or insertions introduced into the polypeptide shown SEQ ID NO: 102 is up to 10, e.g., 1, 2, 3, 4, 5, 6, 7, 8, 9, or 10.

In another embodiment, the present invention relates to variants of the polypeptide shown in SEQ ID NO: 105 comprising a substitution, deletion, and/or insertion at one or more positions. In an embodiment, the number of amino acid substitutions, deletions and/or insertions introduced into the polypeptide shown SEQ ID NO: 105 is up to 10, e.g., 1, 2, 3, 4, 5, 6, 7, 8, 9, or 10.

In another embodiment, the present invention relates to variants of the polypeptide shown in SEQ ID NO: 108 comprising a substitution, deletion, and/or insertion at one or more positions. In an embodiment, the number of amino acid substitutions, deletions and/or insertions introduced into the polypeptide shown SEQ ID NO: 108 is up to 10, e.g., 1, 2, 3, 4, 5, 6, 7, 8, 9, or 10.

The amino acid changes in any of the embodiments above or elsewhere herein may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tag, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for peptidoglycan degradation activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Peptidoglycan Degradation Activity

A polypeptide having peptidoglycan degradation activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly. In one aspect, the polypeptide is an Alicyclobacillus polypeptide. In one aspect, the polypeptide is a Tumebacillus polypeptide. In one aspect, the polypeptide is a Halomonas polypeptide. In one aspect, the polypeptide is a Kribbella polypeptide, e.g., a polypeptide obtained from Kribbella aluminosa. In one aspect, the polypeptide is a Streptomyces polypeptide, e.g., a polypeptide obtained from Streptomyces griseus. In one aspect, the polypeptide is a Nonomuraea polypeptide, e.g., a polypeptide obtained from Nonomuraea coxensis, Nonomuraea dietziae or Nonomuraea guangzhouensis. In one aspect, the polypeptide is a Micromonospora polypeptide, e.g., a polypeptide obtained from Micromonospora peuceia, Micromonospora fulvopurpurea or Micromonospora maritima. In one aspect, the polypeptide is a Laceyella polypeptide, e.g., a polypeptide obtained from Laceyella sacchari. In one aspect, the polypeptide is a Bacillus polypeptide, e.g., a polypeptide obtained from Bacillus sporothermodurans or Bacillus cohnii. In one aspect, the polypeptide is a Lysobacter polypeptide, e.g., a polypeptide obtained from Lysobacter antibioticus or Lysobacter capsica. In one aspect, the polypeptide is a Hamadaea polypeptide, e.g., a polypeptide obtained from Hamadaea tsunoensis. In one aspect, the polypeptide is a Paenibacillus polypeptide, e.g., a polypeptide obtained from Paenibacillus pini. In one aspect, the polypeptide is a Thermostaphylospora polypeptide, e.g., a polypeptide obtained from Thermostaphylospora chromogena. In one aspect, the polypeptide is a Pseudomonas polypeptide, e.g., a polypeptide obtained from Pseudomonas peli or Pseudomonas pseudoalcaligenes.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. Coli lac operon, E. Coli trc promoter (Egon et al., 1988, Gene 69: 301-315), Streptomyces coelicolor agarase gene (daga), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natf. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (g&aA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coliribosomal RNA (rmB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase 1, Trichoderma reesei cellobiohydrolase 11, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Huricola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (npr7), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bargene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobactenum, Helicobacter, Ilyobacter, Neissena, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus dausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacterol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacterol. 171: 3583-3585), or transduction (see, e.g., Burke et al., 2001, Proc. Natd. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32:1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dicdonary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecd (Blastomycetes). Since the classification of yeast may change in the future, for the purposes of this invention, yeast shall be defined as described in Biology and Activites of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina Ceriporiopsis caregiea, Ceriporiopsis gilvescens, Ceriporiopsis pannocinta, Ceriporiopsis rivulose, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginose, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6:1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153:163; and Hinnen et al., 1978, Proc. Nat. Acad. Sci. USA 75:1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide.

The present invention also relates to recombinant methods of producing a polypeptide of the present invention, comprising (a) cultivating a recombinant host cell of the present invention capable of expressing the polypeptide under conditions conducive for production of the polypeptide; and optionally, (b) recovering the polypeptide.

One embodiment of the invention relates to a method of producing a polypeptide, wherein the polypeptide is selected from the group consisting of: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO:96, SEQ ID NO: 99, SEQ ID NO: 102, SEQ ID NO: 105, SEQ ID NO: 108, and polypeptides having at least at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% hereto, comprising (a) cultivating a recombinant host cell of the present invention capable of expressing one of the polypeptides under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide. Another option is to use a supernatant in which the polypeptide has been expressed as a source of the polypeptide.

### Formulation of Enzyme in Granules

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono-, di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238216.

The composition(s) of the invention may be formulated as a granule, for example as a co-granule that combines one or more enzymes. Each enzyme will then be present in more granules, securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates is disclosed in WO 2013/188331, which relates to a detergent composition comprising (a) a multi-enzyme co-granule; (b) less than 10 wt % zeolite (anhydrous basis); and (c) less than 10 wt % phosphate salt (anhydrous basis), wherein said enzyme co-granule comprises from 10 to 98 wt % moisture sink component and the composition additionally comprises from 20 to 80 wt % detergent moisture sink component. WO 2013/188331 also relates to a method of treating and/or cleaning a surface, preferably a fabric surface comprising the steps of (i) contacting said surface with the detergent composition in aqueous wash liquor, (ii) rinsing and/or drying the surface.

A multi-enzyme co-granule may comprise an enzyme of the invention and one or more enzymes selected from the group consisting of proteases, lipases, cellulases, xyloglucanases, perhydrolases, peroxidases, lipoxygenases, laccases, hemicellulases, proteases, cellulases, cellobiose dehydrogenases, xylanases, phospholipases, esterases, cutinases, pectinases, mannanases, pectate lyases, keratinases, reductases, oxidases, phenoloxidases, ligninases, pullulanases, tannases, pentosanases, lichenases, glucanases, arabinosidases, hyaluronidases, chondroitinase, amylases, nucleases, hexosaminidases and mixtures thereof.

An embodiment of the invention relates to an enzyme granule/particle comprising the enzyme of the invention. The granule is composed of a core, and optionally one or more coatings (outer layers) surrounding the core. Typically, the granule/particle size, measured as equivalent spherical diameter (volume based average particle size), of the granule is 20-2000 μm, particularly 50-1500 μm, 100-1500 μm or 250-1200 μm.

The core may include additional materials such as fillers, fibre materials (cellulose or synthetic fibers), stabilizing agents, solubilizing agents, suspension agents, viscosity regulating agents, light spheres, plasticizers, salts, lubricants and fragrances.

The core may include binders, such as synthetic polymer, wax, fat, or carbohydrate.

The core may comprise a salt of a multivalent cation, a reducing agent, an antioxidant, a peroxide decomposing catalyst and/or an acidic buffer component, typically as a homogenous blend.

The core may consist of an inert particle with the enzyme absorbed into it, or applied onto the surface, e.g., by fluid bed coating.

The core may have a diameter of 20-2000 μm, particularly 50-1500 μm, 100-1500 μm or 250-1200 μm.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier.

The core of the enzyme granule/particle may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). Examples of enzyme granules with multiple coatings are shown in WO 93/07263 and WO 97/23606.

The coating may be applied in an amount of at least 0.1% by weight of the core, e.g., at least 0.5%, 1% or 5%. The amount may be at most 100%, 70%, 50%, 40% or 30%.

The coating is preferably at least 0.1 μm thick, particularly at least 0.5 μm, at least 1 μm or at least 5 μm. In a particular embodiment, the thickness of the coating is below 100 μm. In a more particular embodiment the thickness of the coating is below 60 μm. In an even more particular embodiment the total thickness of the coating is below 40 μm.

The coating should encapsulate the core unit by forming a substantially continuous layer. A substantially continuous layer is to be understood as a coating having few or no holes, so that the core unit it is encapsulating/enclosing has few or none uncoated areas. The layer or coating should be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight w/w of a salt, e.g., at least 65%, at least 70%, at least 75%, at least 80%, at least 85%, at least 90%, at least 95% or at least 99% by weight w/w.

The salt may be added from a salt solution where the salt is completely dissolved or from a salt suspension wherein the fine particles is less than 50 μm, such as less than 10 μm or less than 5 μm.

The salt coating may comprise a single salt or a mixture of two or more salts. The salt may be water soluble, in particular, having a solubility at least 0.1 grams in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710.

Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e., a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄·7H₂O), zinc sulfate heptahydrate (ZnSO₄·7H₂O), sodium phosphate dibasic heptahydrate (Na₂HPO₄·7H₂O), magnesium nitrate hexahydrate (Mg(NOs)₂(6H₂O)), sodium citrate dihydrate and magnesium acetate tetrahydrate. Preferably the salt is applied as a solution of the salt, e.g., using a fluid bed.

Thus, in a further aspect, the present invention provides a granule, which comprises:
(a) a core comprising an enzyme according to the invention,
(b) optionally a coating consisting of one or more layer(s) surrounding the core; and
(c) preferably the granule is a co-granulate comprising one or more additional enzyme, preferably selected from proteases, amylases, cellulases.

In one embodiment, the present invention provides a granule, which comprises:
(a) a core comprising a polypeptide having peptidoglycan removal activity, wherein the polypeptide comprises the motif N[IV]X[AG][GAS]A[AY][LV]L (SEQ ID NO: 111),
(b) optionally a coating consisting of one or more layer(s) surrounding the core; and
(c) preferably the granule is a co-granulate comprising one or more additional enzyme, preferably selected from proteases, amylases, cellulases.

In one embodiment, the present invention provides a granule, which comprises:
(a) a core comprising a polypeptide having peptidoglycan removal activity, wherein the polypeptide is selected from the group consisting of: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 105, SEQ ID NO: 108 and polypeptides having at least at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% hereto,
(b) optionally a coating consisting of one or more layer(s) surrounding the core; and
(c) preferably the granule is a co-granulate comprising one or more additional enzyme, preferably selected from proteases, amylases, cellulases.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains at least one organic acid, and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Compositions

The present invention also relates to compositions comprising a polypeptide of the present invention. Preferably, the compositions are enriched in such a polypeptide. The term “enriched” indicates that the peptidoglycan degradation activity of the composition has been increased, e.g., with an enrichment factor of at least 1.1.

The compositions may comprise a polypeptide of the present invention as the major enzymatic component, e.g., a mono-component composition. Alternatively, the compositions may comprise multiple enzymatic activities, such as one or more enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The invention relates to cleaning compositions, e.g., detergent compositions comprising peptidoglycan degradation enzyme in combination with one or more additional cleaning composition components. The choice of additional components is within the skill of the artisan and includes conventional ingredients, including the exemplary non-limiting components set forth below.

One aspect of the invention relates to a cleaning composition comprising a polypeptide having peptidoglycan degradation activity, wherein the polypeptide comprises the motif N[IV]X[AG][GAS]A[AY][LV]L (SEQ ID NO: 111), and at least one cleaning component.

One aspect of the invention relates to a cleaning composition comprising:
a) a polypeptide having peptidoglycan degradation activity, wherein the polypeptide is selected from the group consisting of:
   i. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 3,
   ii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 6,
   iii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 9,
   iv. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 12,
   v. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 15,
   vi. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 18,
   vii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 21,
   viii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 24,
   ix. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 27,
   x. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 30,
   xi. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 33,
   xii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 36,
   xiii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 39,
   xiv. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 42,
   xv. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 45,
   xvi. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 48,
   xvii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 51,
   xviii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 54,
   xix. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 57,
   xx. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 60,
   xxi. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 63,
   xxii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 66,
   xxiii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 69,
   xxiv. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 72,
   xxv. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 75,
   xxvi. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 78,
   xxvii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 81,
   xxviii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 84,
   xxix. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 87,
   xxx. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 90,
   xxxi. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 93,
   xxxii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 96,
   xxxiii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 99,
   xxxiv. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 102,
   xxxv. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 105, and
   xxxvi. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 108, and
b) at least one cleaning component, preferably selected from surfactants, builders, bleach components, polymers, dispersing agents and additional enzymes.

One embodiment relates to a cleaning composition comprising:
a) a polypeptide having peptidoglycan removal activity and which comprises the motif N[IV]X[AG][GAS]A[AY][LV]L (SEQ ID NO: 111), wherein the polypeptide is selected from the group consisting of: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 105, SEQ ID NO: 108 and polypeptides having at least at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% hereto; and
b) at least one cleaning component, preferably selected from surfactants, builders, bleach components, polymers, dispersing agents and additional enzymes.

The peptidoglycan degradation enzyme may be included in the cleaning composition of the present invention at a level of at least 0.0001 to at least 100, at least 0.001 to at least 100, at least 0.01 to at least 100, at least 0.02 to at least 100, at least 0.01 to at least 100, at least 0.1 to at least 100, at least 0.2 to at least 100, at least 0.5 to at least 100 mg/mL, preferably, the concentration of peptidoglycan degradation enzyme in the cleaning composition, e.g., detergent is in the range 0.01 to 100, 0.1 to 50 or 1 to 10 mg/ml. Thus, the detergent composition may comprise at least 0.00008%, preferably at least 0.002%, 0.003%, 0.004%, 0.005%, 0.006%, 0.008%, 0.01%, 0.02%, 0.03%, 0.05%, 0.1%, 0.2%, 0.3%, 0.4%, 0.6%, 0.7%, 0.8%, 0.9% or 1.0% of peptidoglycan degradation enzyme protein.

The choice of cleaning components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

### Surfactants

The cleaning composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 1% to 70% by weight, such as about 1 wt % to about 40 wt %, or about 3 wt % to about 20 wt %, or about 3 wt % to about 10 wt %.

The surfactant(s) is chosen based on the desired cleaning application, and may include any conventional surfactant(s) known in the art.

When included therein the detergent will usually contain from about 1% to about 70% by weight of an anionic surfactant, such as from about 5 wt % to about 50 wt %, including from about 5 wt % to about 20 wt %, or from about 15 wt % to about 20 wt %, or from about 20 wt % to about 25 wt % or at least 30 wt %, at least 40 wt % or at least 50 wt % of an anionic surfactant. Non-limiting examples of anionic surfactants include sulfates and sulfonates, in particular, alkylbenzenesulfonates, such as linear alkybenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weigh of a cationic surfactant, for example from about 0.5% to about 30%, in particular, from about 1% to about 20%, from about 3% to about 10%, such as from about 3% to about 5%, from about 8% to about 12% or from about 10% to about 12%. Non-limiting examples of cationic surfactants include alkykdimethylethanolamine quat (ADMEAQ), cetyltrimethylammonium bromide (CTAB), dimethykdistearylammonium chloride (DSDMAC), and alkylbenzyldimethylammonium, alkyl quatemary ammonium compounds, alkoxylated quatemary ammonium (AQA) compounds, ester quats, and combinations thereof.

When included therein, the detergent will usually contain from about 0.2% to about 40% by weight of a nonionic surfactant, for example from about 0.5 wt % to about 30 wt %, in particular from about 1 wt % to about 20 wt %, from about 3 wt % to about 10 wt %, such as from about 3% wt to about 5 wt %, from about 8 wt % to about 12 wt %, or from about 10 wt % to about 12 wt %. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.01% to about 10% by weight of a semipolar surfactant. Non-limiting examples of semipolar surfactants include amine oxides (AO) such as alkyldmethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 0.01% to about 10% by weight of a zwitterionic surfactant. Non-limiting examples of zwitterionic surfactants include betaines such as alkyldmethylbetaines, sulfobetaines, and combinations thereof.

Typically, more than one surfactant is present in the cleaning composition, e.g., at least one anionic and at least one non-ionic surfactant. Preferably the amount of all surfactant present (total amount) i.e., the amount of anionic, non-ionic, zwitterionic and cationic surfactant present is preferably from about 1 wt % to 80 wt % by weight, such as about 1 wt % to 70 wt %, such as about 1 wt % to 50 wt % such as about 1 wt % to about 40 wt %, or about 5 wt % to about 40 wt %, or about 10 wt % to about 60 wt %. The ratio between the surfactants present depends on the specific composition but the weight ratios may be when an anionic and non-ionic surfactant is included in the composition a weight ratio of the anionic to nonionic surfactant from; 30:1 to 10:1, 20:1 to 1:10, 25:1 to 1-2, 20:1 to 1:5.

One embodiment relates to a cleaning composition comprising a peptidoglycan degrading enzyme, preferably having N-acetylmuramyl-L-alanine amidase and peptidoglycan lyase activity and wherein the cleaning component is at least one surfactant, preferably anionic and/or nonionic, preferably wherein the composition comprises from 1 to 70 wt %, preferably from 5 to 40 wt % surfactant, wherein the surfactant preferably is selected from alkylbenzenesulfonates, e.g., LAS, alkyl sulfates (AS) and mixtures thereof, preferably the cleaning composition comprises at least 20 wt % alkylbenzenesulfonate surfactant.

One embodiment relates to a cleaning composition comprising a peptidoglycan degrading enzyme, preferably having N-acetylmuramyl-L-alanine amidase and peptidoglycan lyase activity, wherein the cleaning composition comprises at least one anionic surfactant and wherein the cleaning composition additionally comprises a nonionic surfactant, and preferably wherein the weight ratio of the anionic to nonionic surfactant is from 25:1 to 1:2 or from 1.5:1 to 1:10.

### Builders and Co-Builders

The cleaning composition may contain about 0-65% by weight, such as about 5% to about 50%, such as about 0.5% to about 20% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis-(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 2009/102854, U.S. Pat. No. 5,977,053.

### Bleaching Systems

The cleaning composition may contain 0-30% by weight, such as about 1% to about 20%, such as about 0.01% to about 10% of a bleaching system. Any bleaching system comprising components known in the art for use in cleaning detergents may be utilized. Suitable bleaching system components include sources of hydrogen peroxide; sources of peracids; and bleach catalysts or boosters.

### Sources of Hydrogen Peroxide:

Suitable sources of hydrogen peroxide are inorganic persalts, including alkali metal salts such as sodium percarbonate and sodium perborates (usually mono- or tetrahydrate), and hydrogen peroxide-urea (1/1).

### Sources of Peracids:

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.
a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.
b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO 98/17767. A particular family of bleach activators of interest was disclosed in EP 624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

### Bleach Catalysts and Boosters

The bleaching system may also include a bleach catalyst or booster. Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese(III). The bleach catalysts may also be other metal compounds; such as iron or cobalt complexes.

In some embodiments, where a source of a peracid is included, an organic bleach catalyst or bleach booster may be used having one of the following formulae:
(i)

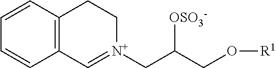

(ii)

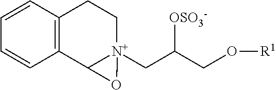

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described in, e.g., WO 2007/087258, WO 2007/087244, WO 2007/087259, EP 1867708 (Vitamin K) and WO 2007/087242. Suitable photobleaches may for example be sulfonated zinc or aluminum phthalocyanines.

### Metal Care Agents

Metal care agents may prevent or reduce the tarnishing, corrosion or oxidation of metals, including aluminium, stainless steel and non-ferrous metals, such as silver and copper. Suitable examples include one or more of the following:
(a) benzatriazoles, including benzotriazole or bis-benzotriazole and substituted derivatives thereof. Benzotriazole derivatives are those compounds in which the available substitution sites on the aromatic ring are partially or completely substituted. Suitable substituents include linear or branch-chain Ci-C20- alkyl groups (e.g., C1-C20- alkyl groups) and hydroxyl, thio, phenyl or halogen such as fluorine, chlorine, bromine and iodine.
(b) metal salts and complexes chosen from the group consisting of zinc, manganese, titanium, zirconium, hafnium, vanadium, cobalt, gallium and cerium salts and/or complexes, the metals being in one of the oxidation states II, Ill, IV, V or VI. In one aspect, suitable metal salts and/or metal complexes may be chosen from the group consisting of Mn(II) sulphate, Mn(II) citrate, Mn(II) stearate, Mn(II) acetylacetonate, K\hat{ }TiF6 (e.g., K2TiF6), K\hat{ }ZrF6 (e.g., K2ZrF6), CoSO4, Co(NOs)2 and Ce(NOs)3, zinc salts, for example zinc sulphate, hydrozincite or zinc acetate;
(c) silicates, including sodium or potassium silicate, sodium disilicate, sodium metasilicate, crystalline phyllosilicate and mixtures thereof.

Further suitable organic and inorganic redox-active substances that act as silver/copper corrosion inhibitors are disclosed in WO 94/26860 and WO 94/26859. Preferably, the composition of the invention comprises from 0.1 to 5% by weight of the composition of a metal care agent, preferably the metal care agent is a zinc salt.

### Hydrotropes

The cleaning composition may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Polymers

The cleaning composition may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Suitable examples include PVP-K15, PVP-K30, ChromaBond S-400, ChromaBond S-403E and Chromabond S-100 from Ashland Aqualon, and SokalanM HP 165, SokalanO HP 50 (Dispersing agent), Sokalan® HP 53 (Dispersing agent), SokalanM HP 59 (Dispersing agent), SokalanS HP 56 (dye transfer inhibitor), SokalanO HP 66 K (dye transfer inhibitor) from BASF. Further exemplary polymers include sulfonated polycarboxylates, polyethylene oxide and polypropylene oxide (PEO-PPO) and diquatemium ethoxy sulfate. Other exemplary polymers are disclosed in, e.g., WO 2006/130575. Salts of the above-mentioned polymers are also contemplated. Particularly preferred polymer is ethoxylated homopolymer SokalanM HP 20 from BASF, which helps to prevent redeposition of soil in the wash liquor.

### Fabric Hueing Agents

The cleaning compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/003274, WO 2005/003275, WO 2005/003276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g., WO 2007/087257 and WO 2007/087243.

### Dispersants

The cleaning compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The cleaning compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The cleaning compositions of the present invention will preferably also contain additional components that may tint articles being cleaned, such as fluorescent whitening agents or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostibene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stibene-2,2′-disulfonate and sodium 5-(2H-naphtho[1,2-d][1,2,3]triazol-2-yl)-2-[(E)-2-phenylvinyl]-benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

### Soil Release Polymers

The cleaning compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers. Suitable graft co-polymers are described in more detail in WO 2007/138054, WO 2006/108856 and WO 2006/113314 (hereby incorporated by reference). Suitable polyethylene glycol polymers include random graft co-polymers comprising: (i) hydrophilic backbone comprising polyethylene glycol; and (ii) side chain(s) selected from the group consisting of: C4-C25 alkyl group, polypropylene, polybutylene, vinyl ester of a saturated C1-C6 mono-carboxylic acid, CI-C 6 alkyl ester of acrylic or methacrylic acid, and mixtures thereof. Suitable polyethylene glycol polymers have a polyethylene glycol backbone with random grafted polyvinyl acetate side chains. The average molecular weight of the polyethylene glycol backbone can be in the range of from 2,000 Da to 20,000 Da, or from 4,000 Da to 8,000 Da. The molecular weight ratio of the polyethylene glycol backbone to the polyvinyl acetate side chains can be in the range of from 1:1 to 1:5, or from 1:1.2 to 1-2. The average number of graft sites per ethylene oxide units can be less than 1, or less than 0.8, the average number of graft sites per ethylene oxide units can be in the range of from 0.5 to 0.9, or the average number of graft sites per ethylene oxide units can be in the range of from 0.1 to 0.5, or from 0.2 to 0.4. A suitable polyethylene glycol polymer is Sokalan HP22. Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The cleaning compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The cleaning compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable cleaning composition components include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Polymers

The cleaning composition may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquatemium ethoxy sulfate. Other exemplary polymers are disclosed in, e.g., WO 2006/130575. Salts of the above-mentioned polymers are also contemplated. Particularly preferred polymer is ethoxylated homopolymer Sokalan® HP 20 from BASF, which helps to prevent redeposition of soil in the wash liquor.

### Enzymes

The cleaning composition may comprise one or more additional enzymes such as one or more lipase, cutinase, an amylase, carbohydrase, cellulase, pectinase, mannanase, arabinase, galactanase, xylanase, oxidase, e.g., a laccase, and/or peroxidase. In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Cellulases

Suitable cellulases include complete cellulases or mono-component endoglucanases of bacterial or fungal origin. Chemically or genetically modified mutants are included. The cellulase may for example be a mono-component or a mixture of mono-component endo-1,4-beta-glucanase often just termed endoglucanases. Suitable cellulases include a fungal cellulase from Humicola insolens (U.S. Pat. No. 4,435,307) or from Trichoderma, e.g., T. reesei or T. viride. Examples of cellulases are described in EP 495257. Other suitable cellulases are from Thielavia, e.g., Thielavia terrestris as described in WO 96/29397 or Fusarium oxysporum as described in WO 91/17244 or from Bacillus as described in, WO 02/099091 and JP 2000210081. Other examples are cellulase variants such as those described in WO 94/07998, EP 531315, U.S. Pat. Nos. 5,457,046, 5,686,593, 5,763,254, WO 95/24471, WO 98/12307 Commercially available cellulases include Carezyme®, Celluzyme®, Celluclean®, Celluclast® and Endolase®; Renozyme®; Whitezyme® (Novozymes A/S) Puradax®, Puradax HA, and Puradax EG (available from Genencor).

### Proteases

Suitable proteases may be of any origin, but are preferably of bacterial or fungal origin, optionally in the form of protein engineered or chemically modified mutants. The protease may be an alkaline protease, such as a serine protease or a metalloprotease. A serine protease may for example be of the S1 family, such as trypsin, or the S8 family such as a subtilisin. A metalloprotease may for example be a thermolysin, e.g., from the M4 family, or another metalloprotease such as those from the M5, M7 or M35 families.

The term “subtilases” refers to a sub-group of serine proteases according to Siezen et al., 1991, Protein Eng. 4: 719-737 and Siezen et al., 1997, Protein Sci. 6: 501-523. Serine proteases are a subgroup of proteases characterized by having a serine in the active site, which forms a covalent adduct with the substrate. The subtilases may be divided into six subdivisions, the Subtilisin family, the Thermitase family, the Proteinase K family, the Lantibiotic peptidase family, the Kexin family and the Pyrolysin family.

Although proteases suitable for detergent use may be obtained from a variety of organisms, including fungi such as Aspergillus, detergent proteases have generally been obtained from bacteria and in particular from Bacillus. Examples of Bacillus species from which subtilases have been derived include Bacillus lentus, Bacillus alkalophilus, Bacillus subtilis, Bacillus amyloliquefaciens, Bacillus licheniformis, Bacillus punmilus and Bacillus gibsonii. Particular subtilisins include subtilisin lentus, subtilisin Novo, subtilisin Carlsberg, subtilisin BPN′, subtilisin 309, subtilisin 147 and subtilisin 168 and, e.g., protease PD138 (described in WO 93/18140). Other useful proteases are, e.g., those described in WO 01/16285 and WO 02/16547.

Examples of trypsin-like proteases include the Fusarium protease described in WO 94/25583 and WO 2005/040372, and the chymotrypsin proteases derived from Cellumonas described in WO 2005/052161 and WO 2005/052146.

Examples of metalloproteases include the neutral metalloproteases described in WO 2007/044993 such as those derived from Bacillus amyloliquefaciens, as well as, e.g., the metalloproteases described in WO 2015/158723 and WO 2016/075078.

Examples of useful proteases are the protease variants described in WO 89/06279 WO 92/19729, WO 96/34946, WO 98/20115, WO 98/20116, WO 99/11768, WO 01/44452, WO 2003/006602, WO 2004/003186, WO 2004/041979, WO 2007/006305, WO 2011/036263, WO 2014/207227, WO 2016/087617 and WO 2016/174234. Preferred protease variants may, for example, comprise one or more of the mutations selected from the group consisting of: S3T, V4I, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V1021, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, Al88P, G189E, V193M, N198D, V1991, Q200L, Y203W, S206G, L211Q, L211D, N212D, N212S, M216S, A226V, K229L, Q230H, Q239R, N246K, S253D, N255W, N255D, N255E, L256E, L256D T268A and R269H, wherein position numbers correspond to positions of the Bacillus lentus protease shown in SEQ ID NO: 1 of WO 2016/001449. Protease variants having one or more of these mutations are preferably variants of the Bacillus lentus protease (Savinase®, also known as subtilisin 309) shown in SEQ ID NO: 1 of WO 2016/001449 or of the Bacillus amyloliquefaciens protease (BPN′) shown in SEQ ID NO: 2 of WO 2016/001449. Such protease variants preferably have at least 80% sequence identity to SEQ ID NO: 1 or to SEQ ID NO: 2 of WO 2016/001449.

Another protease of interest is the alkaline protease from Bacillus lentus DSM 5483, as described for example in WO 91/02792, and variants thereof which are described for example in WO 92/21760, WO 95/23221, EP 1921147, EP 1921148 and WO 2016/096711.

The protease may alternatively be a variant of the TY145 protease having SEQ ID NO: 1 of WO 2004/067737, for example a variant comprising a substitution at one or more positions corresponding to positions 27, 109, 111, 171, 173, 174, 175, 180, 182, 184, 198, 199 and 297 of SEQ ID NO: 1 of WO 2004/067737, wherein said protease variant has a sequence identity of at least 75% but less than 100% to SEQ ID NO: 1 of WO 2004/067737. TY145 variants of interest are described in, e.g., WO 2015/014790, WO 2015/014803, WO 2015/014804, WO 2016/097350, WO 2016/097352, WO 2016/097357 and WO 2016/097354.

Examples of preferred proteases include:
(a) variants of SEQ ID NO: 1 of WO 2016/001449 comprising two or more substitutions selected from the group consisting of S9E, N43R, N76D, Q206L, Y209W, S259D and L262E, for example a variant with the substitutions S9E, N43R, N76D, V2051, Q206L, Y209W, S259D, N261W and L262E, or with the substitutions S9E, N43R, N76D, N185E, S188E, Q191N, A194P, Q206L, Y209W, S259D and L262E, wherein position numbers are based on the numbering of SEQ ID NO: 2 of WO 2016/001449;
(b) a variant of the polypeptide of SEQ ID NO: 1 of WO 2016/001449 with the mutation S99SE, wherein position numbers are based on the numbering of SEQ ID NO: 2 of WO 2016/001449;
(c) a variant of the polypeptide of SEQ ID NO: 1 of WO 2016/001449 with the mutation S99AD, wherein position numbers are based on the numbering of SEQ ID NO: 2 of WO 2016/001449;
(d) a variant of the polypeptide of SEQ ID NO: 1 of WO 2016/001449 with the substitutions Y167A+R170S+A194P, wherein position numbers are based on the numbering of SEQ ID NO: 2 of WO 2016/001449;
(e) a variant of the polypeptide of SEQ ID NO: 1 of WO 2016/001449 with the substitutions S9R+A15T+V68A+N218D+Q245R, wherein position numbers are based on the numbering of SEQ ID NO: 2 of WO 2016/001449;
(f) a variant of the polypeptide of SEQ ID NO: 1 of WO 2016/001449 with the substitutions S9R+A15T+G61E+V68A+A194P+V2051+Q245R+N261D, wherein position numbers are based on the numbering of SEQ ID NO: 2 of WO 2016/001449;
(g) a variant of the polypeptide of SEQ ID NO: 1 of WO 2016/001449 with the substitutions S99D+S101R/E+S103A+V104I+G160S; for example a variant of SEQ ID NO: 1 of WO 2016/001449 with the substitutions S3T+V41+S99D+S101EE+S103A+V1041+G160S+V205I, wherein position numbers are based on the numbering of SEQ ID NO: 2 of WO 2016/001449;
(h) a variant of the polypeptide of SEQ ID NO: 2 of WO 2016/001449 with the substitutions S24G+S53G+S78N+S101N+G128A/S+Y217Q, wherein position numbers are based on the numbering of SEQ ID NO: 2 of WO 2016/001449;
(i) the polypeptide disclosed in GENESEQP under accession number BER84782, corresponding to SEQ ID NO: 302 in WO 2017/210295;
(j) a variant of the polypeptide of SEQ ID NO: 1 of WO 2016/001449 with the substitutions S99D+S101E+S103A+V104I+S156D+G160S+L262E, wherein position numbers are based on the numbering of SEQ ID NO: 2 of WO 2016/001449;
(k) a variant of the polypeptide of SEQ ID NO: 1 of WO 2016/001449 with the substitutions S9R+A15T+G61E+V68A+N76D+S99G+N218D+Q245R, wherein position numbers are based on the numbering of SEQ ID NO: 2 of WO 2016/001449;
(l) a variant of the polypeptide of SEQ ID NO: 1 of WO 2016/001449 with the substitutions V68A+S106A, wherein position numbers are based on the numbering of SEQ ID NO: 2 of WO 2016/001449; and
(m) a variant of the polypeptide of SEQ ID NO: 1 of WO 2004/067737 with the substitutions S27K+N109K+S111E+S171E+S173P+G174K+S175P+F180Y+G182A+L184F+Q198E+N199+T297P, wherein position numbers are based on the numbering of SEQ ID NO: 1 of WO 2004/067737.

Suitable commercially available protease enzymes include those sold under the trade names Alcalase®, Duralase™, Durazym™, Relase®, Relase® Ultra, Savinase®, Savinase® Ultra, Primase™, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Blaze Evity® 200T, Neutrase®, Everlase®, Esperase®, Progress® Uno, Progress® In and Progress® Excel (Novozymes A/S), those sold under the tradename Maxatase™, Maxacal™, Maxapem®, Purafect® Ox, Purafect® OxP, Puramax®, FN2™, FN3™, FN4^{ex}™, Excellase®, Excellenz™ P1000, Excellenz™ P1250, Eraserrm, Preferenz® P100, Purafect Prime, Preferenz P110™, Effecten™ P1000™, Purafect®, Effecten™ P1050™, Purafect® Ox, Effecten™ P2000, Purafast™, Properase®, Opticlean™ and Optimase® (Danisco/DuPont), BLAP (sequence shown in FIG. 29 of U.S. Pat. No. 5,352,604) and variants hereof (Henkel AG), and KAP (Bacillus alkalophilus subtilisin) from Kao.

### Lipases and Cutinases

Suitable lipases and cutinases include those of bacterial or fungal origin. Chemically modified or protein engineered mutant enzymes are included. Examples include lipase from Thermomyces, e.g., from T. lanuginosus (previously named Humicola lanuginosa) as described in EP 258068 and EP 305216, cutinase from Huricola, e.g., H. insolens (WO 96/13580), lipase from strains of Pseudomonas (some of these now renamed to Burkholderia), e.g., P. alcaligenes or P. pseudoalcaligenes (EP 218272), P. cepacia (EP 331376), P. sp. strain SD705 (WO 95/06720 & WO 96/27002), P. wisconsinensis (WO 96/12012), GDSL-type Streptomyces lipases (WO 2010/065455), cutinase from Magnaporthe grisea (WO 2010/107560), cutinase from Pseudomonas mendocina (U.S. Pat. No. 5,389,536), lipase from Thermobifida fusca (WO 2011/084412), Geobacillus stearothermophilus lipase (WO 2011/084417), lipase from Bacillus subtilis (WO 2011/084599), and lipase from Streptomyces griseus (WO 2011/150157) and S. pristinaespiralis (WO 2012/137147).

Other examples are lipase variants such as those described in EP 407225, WO 92/05249, WO 94/01541, WO 94/25578, WO 95/14783, WO 95/30744, WO 95/35381, WO 95/22615, WO 96/00292, WO 97/04079, WO 97/07202, WO 00/34450, WO 00/60063, WO 01/92502, WO 2007/087508 and WO 2009/109500.

Preferred commercial lipase products include Lipolase™, Lipex™; Upolex™ and Lipoclean™ (Novozymes A/S), Lumafast (originally from Genencor) and Lipomax (originally from Gist-Brocades). Still other examples are lipases sometimes referred to as acyltransferases or perhydrolases, e.g., acyltransferases with homology to Candida antarctica lipase A (WO 2010/111143), acyltransferase from Mycobacterium smegmatis (WO 2005/056782), perhydrolases from the CE 7 family (WO 2009/067279), and variants of the M. smegmatis perhydrolase in particular the S54V variant used in the commercial product Gentle Power Bleach from Huntsman Textile Effects Pte Ltd (WO 2010/100028).

### Amylases

Suitable amylases may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/19467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/10355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:
M197T;
H156Y+A181T+N190F+A209V+Q264S; or
G48A+T49I+G107A+H156Y+A181T+N190F+I201F+A209V+Q264S.

Further amylases which are suitable are amylases having SEQ ID NO: 6 in WO 99/19467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/23873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID NO: 2 of WO 96/23873 for numbering. More preferred variants are those having a deletion in two positions selected from 181, 182, 183 and 184, such as 181 and 182,182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 2008/153815, SEQ ID NO: 10 in WO 01/66712 or variants thereof having 90% sequence identity to SEQ ID NO: 2 of WO 2008/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 2009/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:
N128C+K178L+T182G+Y305R+G475K;
N128C+K178L+T182G+F202Y+Y305R+D319T+G475K;
S125A+N128C+K178L+T182G+Y305R+G475K; or
S125A+N128C+T131I+T165I+K178L+T182G+Y305R+G475K;
   wherein the variants are C-terminally truncated and optionally further comprise a substitution at position 243 and/or a deletion at position 180 and/or position 181.

Further suitable amylases are amylases having SEQ ID NO: 1 of WO 2013/184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:
E187P+I203Y+G476K
E187P+I203Y+R458N+T459S+D460T+G476K
   wherein the variants optionally further comprise a substitution at position 241 and/or a deletion at position 178 and/or position 179.

Further suitable amylases are amylases having SEQ ID NO: 1 of WO 2010/104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of 1181 and/or G182. Most preferred amylase variants of SEQ ID NO: 1 are those having the substitutions:
N21D+D97N+V128I,
   wherein the variants optionally further comprise a substitution at position 200 and/or a deletion at position 180 and/or position 181.

Other suitable amylases are the alpha-amylase having SEQ ID NO: 12 in WO 01/66712 or a variant having at least 90% sequence identity to SEQ ID NO: 12. Preferred amylase variants are those having a substitution, a deletion or an insertion in one of more of the following positions of SEQ ID NO: 12 in WO 01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO 2011/098531, WO 2013/001078 and WO 2013/001087.

Commercially available amylases are Duramy™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase, Preferen™ S1000, Preferen™ S100, Preferen™ S110 and Preferen™ S210 (from Genencor International Inc./DuPont).

### Peroxidases/Oxidases

A peroxidase may be an enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity. Suitable peroxidases include those of plant, bacterial or fungal origin. Chemically modified or protein engineered mutants are included. Examples of useful peroxidases include peroxidases from Coprinopsis, e.g., from C. cinerea (EP 179486), and variants thereof as those described in WO 93/24618, WO 95/10602, and WO 98/15257. A peroxidase may also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. The haloperoxidase may be a chloroperoxidase. Preferably, the haloperoxidase is a vanadium haloperoxidase, i.e., a vanadate-containing haloperoxidase. In a preferred method the vanadate-containing haloperoxidase is combined with a source of chloride ion. Haloperoxidases have been isolated from many different fungi, in particular from the fungus group dematiaceous hyphomycetes, such as Caldariomyces, e.g., C. fumago, Altemara, Curvularia, e.g., C. verruculosa and C. inaequalis, Drechslera, Ulocladium and Botrytis. Haloperoxidases have also been isolated from bacteria such as Pseudomonas, e.g., P. pyrrocinia and Streptomyces, e.g., S. aureofaciens. The haloperoxidase may be derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeolrichoconis crotalarie as described in WO 01/79461, or Geniculosporium sp. as described in WO 01/79460.

Oxidases include any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5). Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g., N. crassa, Podospora, Borytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila Schytalidium, e.g., S. thermophilum, Polyporus, e.g., P. pinsitus, Phlebia, e.g., P. radiata (WO 92/01046), or Coriolus, e.g., C. hirsutus (JP 2238885). Suitable examples from bacteria include a laccase derivable from a strain of Bacillus. A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Microorganisms

The detergent additive as well as the detergent composition may also comprise one or more microorganisms, such as one or more fungi, yeast, or bacteria. In an embodiment, the one or more microorganisms are dehydrated (for example by lyophilization) bacteria or yeast, such as a strain of Lactobacillus. In another embodiment, the microorganisms are one or more microbial spores (as opposed to vegetative cells), such as bacterial spores; or fungal spores, conidia, hypha. Preferably, the one or more spores are Bacillus endospores; even more preferably the one or more spores are endospores of Bacillus subtilis, Bacillus licheniformis, Bacillus amyloliquefaciens, or Bacillus megaterum. The microorganisms may be included in the detergent composition or additive in the same way as enzymes (see above).

### Formulation of Detergent Products

The cleaning composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations. In a specific aspect, the present invention provides a detergent additive comprising one or more enzymes as described herein. The cleaning composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for holding the composition, e.g., without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water-soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water-soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water-soluble film. The compartment for liquid components can be different in composition than compartments containing solids: US 2009/0011970.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

### Uses

The present invention is also directed to methods for using the compositions thereof. Laundry/textile/fabric (Household laundry washing, Industrial laundry washing). Hard surface cleaning (ADW, car wash, Industrial surface).

### Use of Cleaning Composition

The detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations. In a specific aspect, the present invention provides a detergent additive comprising one or more enzymes as described herein.

### Methods

The invention further relates to a method of treating a method of treating a fabric comprising:
(a) contacting the fabric with an aqueous solution of peptidoglycan degradation enzyme, preferably having N-acetylmuramyl-L-alanine amidase and peptidoglycan lyase activity; and optionally
(b) rinsing and drying the textile.

The invention further relates to a method for cleaning or laundering an item comprising the steps of:
a. exposing an item to a wash liquor comprising at least one peptidoglycan degradation enzyme, preferably having N-acetylmuramyl-L-alanine amidase and/or peptidoglycan lyase activity or a detergent composition comprising such enzyme;
b. completing at least one wash cycle; and optionally
c. rinsing the item,
   wherein the item is a fabric.

The invention further relates to a method for cleaning or laundering an item comprising the steps of:
a. exposing an item to a wash liquor comprising a polypeptide or a detergent composition comprising a polypeptide, preferably wherein the polypeptide has N-acetylmuramyl-L-alanine amidase and/or peptidoglycan lyase activity;
b. completing at least one wash cycle; and optionally
c. rinsing the item,
   wherein the item is a fabric.

The invention further relates to a method for cleaning or laundering an item comprising the steps of:
a. exposing an item to a wash liquor comprising a polypeptide or a detergent composition comprising a polypeptide, preferably wherein the polypeptide has N-acetylmuramyl-L-alanine amidase and/or peptidoglycan lyase activity and wherein the polypeptide comprises the motif N[IV]X[AG][GAS]A[AY][LV]L (SEQ ID NO: 111);
b. completing at least one wash cycle; and optionally
c. rinsing the item,
   wherein the item is a fabric.

The invention further relates to a method for cleaning or laundering an item comprising the steps of:
a. exposing an item to a wash liquor comprising a polypeptide or a detergent composition comprising a polypeptide, preferably wherein the is selected from the group consisting of: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 105, SEQ ID NO: 108 and polypeptides having at least at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% hereto;
b. completing at least one wash cycle; and optionally
c. rinsing the item,
   wherein the item is a fabric.

The pH of the liquid solution is in the range of 1 to 11, such as in the range 5.5 to 11, such as in the range of 7 to 9, in the range of 7 to 8 or in the range of 7 to 8.5.

The wash liquor may have a temperature in the range of 5° C. to 95° C., or in the range of 10° C. to 80° C., in the range of 10° C. to 70° C., in the range of 10° C. to 60° C., in the range of 10° C. to 50° C., in the range of 15° C. to 40° C. or in the range of 20° C. to 30° C. In one aspect, the temperature of the wash liquor is 30° C.

The concentration of the peptidoglycan degradation enzyme in the wash liquor is typically in the range of at least 0.00001 ppm to at least 10 ppm, at least 0.00002 ppm to at least 10 ppm, at least 0.0001 ppm to at least 10 ppm, at least 0.0002 ppm to at least 10 ppm, at least 0.001 ppm to at least 10 ppm, at least 0.002 ppm to at least 10 ppm, at least 0.01 ppm to at least 10 ppm, at least 0.02 ppm to at least 10 ppm, at least 0.1 ppm to at least 10 ppm, at least 0.2 ppm to at least 10 ppm, at least 0.5 ppm to at least 5 ppm.

The present invention is further described by the following examples that should not be construed as limiting the scope of the invention.

### EXAMPLES

### Media and Solutions

Model detergent A: 12 wt % LAS, 1.1 wt % AEO Biosoft N25-7 (NI), 7 wt % AEOS (SLES), 6 wt % MPG, 3 wt % ethanol, 3 wt % TEA (triethanolamine), 2.75 wt % cocoa soap, 2.75 wt % soya soap, 2 wt % glycerol, 2 wt % sodium hydroxide, 2 wt % sodium citrate, 1 wt % sodium formiate, 0.2 wt % DTMPA, 0.2 wt % PCA.

Model detergent O: 4 wt % sodium dodecylbenezenesulfonate (LAS), 8 wt % sodium lauryl ether sulfate (SLES/AEOS), 1 wt % soap (soy fatty acid), 4 wt % alcohol ethoxylate (AEO), 0.4 wt % triethanolamine (TEA), 2 wt % sodium citrate, 0.02 wt % calcium chloride dihydrate.

For Example 4, a wash liquor of model detergent A was prepared by dissolving 3.33 g/l of the detergent in water with a hardness of 150 dH.

For Example 5, 2.67 g/I model 0 and 0.44 g/I model A, respectively, were dissolved in tap water.

### Assays

### Peptidoglycan-Degrading Activity Measurement

The peptidoglycan-degrading activity was estimated using the Invitrogen™ EnzChek™ Lysozyme Assay Kit (ThermoFisher, E22013) as recommended by the manufacturer. The DQ™ substrate supplied with the kit was dissolved in miliQ-H₂O to yield a 1.0 mg/ml substrate stock solution. This solution was further diluted to 50 μg/ml by mixing 50 μl stock substrate solution with 950 μl 1× Reaction buffer supplied with the kit. Concentrated enzyme solution was diluted to 2 μg/ml in the 1× Reaction buffer. 50 μl of the 50 μg/ml substrate solution was mixed with either 50 μl 1× Reaction buffer or 50 μl 2 μg/ml enzyme solution to yield a final enzyme concentration in the reaction of 1 μg/ml. The sample was incubated at 37° C. and fluorescence development was measured using a POLARstar Omega plate reader spectrophotometer (BMG LABTECH) with an excitation wavelength of 485 nm emission wavelength of 520 nm and a gain of 1500.

Fluorescence units were plotted against time and the initial slope was estimated. The results are given in the table below. Clear peptidoglycan-degrading enzyme activity is observed for the enzyme.

| Enzyme | Initial slope (fluorescence units/min) |
|---|---|
| No enzyme | −189.64 |
| SEQ ID NO: 6 | 13228 |

### Example 1: Cloning and Expression of Polypeptides: Strains and DNA

DNA encoding the genes of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103 and SEQ ID NO: 106 was isolated from bacterial strains and environmental bacterial communities isolated from soil samples collected in different countries (see Table 1). Chromosomal DNA from the different strains and bacterial communities was subjected to full genome sequencing using Illumina technology. The genome sequences were analyzed for protein sequences that contained an Amidase_2 domain, as defined in PFAM (PF01510, Pfam version 31.0 Finn (2016). Nucleic Acids Research, Database Issue 44: D279-D285).

TABLE 1

| Enzyme | Donor | Country of origin |
|---|---|---|
| SEQ | Alicyclobacillus sp. | Denmark |
| ID NO: | | |
| 3 | Hamadaea tsunoensis | Japan |
| 6 | Micromonospora maritima | United States |
| 9 | Paenibacillus sp. | United States |
| 12 | Nonomuraea sp. | United Kingdom |
| 15 | Lysobacter antibioticus | China |
| 18 | Micromonospora sp. | United Kingdom |
| 21 | Nonomuraea coxensis | Philippines 1990 |
| 24 | Micromonospora fulvopurpurea | unknown strain |
| | | isolated 1970 |
| 27 | Alicyclobacillus sp. | Denmark |
| 30 | Halomonas sp. | United States |
| 33 | Pseudomonas peli | United States |
| 36 | Halomonas sp. | United States |
| 39 | Pseudomonas pseudoalcaligenes | United States |
| 42 | Tumebacillus sp. | United States |
| 45 | Nonomuraea dietziae | United Kingdom |
| 48 | Laceyella sacchari | Denmark |
| 51 | Thermostaphylospora | Unknown, date of sampling |
| | chromogena | 22 Aug. 1990 |
| 54 | Kribbella aluminosa | China |
| 57 | Streptomyces griseus | United States |
| 60 | Micromonospora peucetia | United Kingdom |
| 63 | Bacillus sp. | Japan |
| 66 | Bacillus sporothermodurans | Denmark |
| 69 | Paenibacillus pini | Sweden |
| 72 | Bacillus cohnii | United States |
| 75 | Kribbella sp. | United Kingdom |
| 78 | Bacillus sp. | United States |
| 81 | Bacillus sp. | United States |
| 84 | Bacillus sp. | United States |
| 87 | Streptomyces sp. | China |
| 90 | Bacillus sp. | United States |
| 93 | Bacillus sp. | United States |
| 96 | Nonomuraea guangzhouensis | United Kingdom |
| 99 | Nonomuraea guangzhouensis | United Kingdom |
| 102 | Bacillus cohnii | Denmark |
| 105 | Halomonas sp. | United States |
| 108 | Lysobacter capsica | United States |

### Example 2: Cloning and Expression of Polypeptides of the Invention

DNA encoding the mature peptides of peptidoglycan degradation enzyme genes SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103 and SEQ ID NO: 106 was amplified from the genomic DNA of the corresponding bacterial strains by standard PCR techniques using specific primers containing an overhang to cloning vector. The amplified PCR fragments were inserted into a Bacillus expression vector as described in WO 2012/025577. Briefly, the DNA encoding the mature peptide of the gene was cloned in frame to a Bacillus clausii secretion signal (BcSP; with the following amino acid sequence: MKKPLGKIVASTALLISVAFSSSIASA (SEQ ID NO: 109). BcSP replaced the native secretion signal in the gene. Downstream of the BcSP sequence, an affinity tag sequence was introduced to ease the purification process (His-tag; with the following amino acid sequence: HHHHHHPR (SEQ ID NO: 110) The gene that was expressed therefore comprised the BcSP sequence followed by the His-tag sequence followed by the mature wild type gene sequence. The final expression plasmid (BcSP-His-tag-PGLGene) was transformed into a Bacillus subtilis expression host. The BcSP-fusion gene was integrated by homologous recombination into the Bacillus subtilis host cell genome upon transformation. The gene construct was expressed under the control of a triple promoter system (as described in WO 99/43835). The gene coding for chloramphenicol acetyltransferase was used as maker (as described in (Diderichsen et al., 1993, Plasmid 30: 312-315)). Transformants were selected on LB media agar supplemented with 6 microgram of chloramphenicol per ml. One recombinant Bacillus subtilis clone containing the PGL expression construct was selected and was cultivated on a rotary shaking table in 500 ml baffled Erlenmeyer flasks each containing 100 ml yeast extract-based media. After 3-5 days' cultivation time at 30° C. to 37° C., the enzyme-containing supernatant was harvested by centrifugation and the enzymes were purified by His-tag purification.

### Example 3: His Tag Purification Method

The His-tagged enzymes were purified by immobilized metal chromatography (IMAC) using Ni²⁺ as the metal ion on 5 mL HisTrap Excel columns (GE Healthcare Life Sciences). The purification took place at pH 7 and the bound protein was eluted with imidazole. The purity of the purified enzymes was checked by SDS-PAGE and the concentration of the enzyme determined by Absorbance 280 nm after a buffer exchange in 50 mM HEPES, 100 mM NaCl pH 7.0.

### Example 4: Attachment of Micrococcus luteus

M. luteus is taken from −80° C. frozen stock and grown on TSA plates for 3 days. From here one colony is inoculated per 10 ml TSB glass tube, whirl mixed and incubated over night at 30° C. with 200 rpm shaking. Then the bacteria are transferred to 50 ml Falcon tubes at 3000 rpm 20° C. for 5 min in a Sorvall centrifuge. The supernatant is removed and re-suspended in 10 ml PBS per tube. Cells are washed twice, added to a 50 ml tube and mixed. A solution of the culture is made with an OD600 of 0.5 as measured in a CLARIOstar® reader. 100 mL is prepared and kept under constant stirring throughout the test.

A detergent solution containing 3.33 g/L model A detergent is prepared by mixing 0.167 g Model A detergent with 50 mL of tap water in a 100 mL BlueCap bottle, stirring for 2-5 min before use. This solution is used for the first and last rows in the setup where no bacteria is added (see below). A detergent solution with bacteria is made by preparing a tap water solution containing M. luteus with an OD600 of 0.5 and 0.33 g Model A detergent, stirring for 2-5 min. This is used as a mix for wells of 48-well plates (Thermo Scientific, Nunc A/S, non-treated, PS, sterile, cat.no. 150787). A setup of blanks and enzymes is created so as to randomize the positions for repetitions in the plates to account for the systematic molding variation. An example may look like the following:

| | 1 | 2 | 3 | 4 | 5 | 6 | 7 | 8 |
|---|---|---|---|---|---|---|---|---|
| | No bacteria | bacteria | bacteria | bacteria | bacteria | bacteria | bacteria | No bacteria |
| | added | added | added | added | added | added | added | added |
| A | Control, no | Blank, no | Enz1 | Enz3 | Enz2 | Enz4 | Ref | Control, no |
| | enzyme | enzyme | | | | | enzyme | enzyme |
| B | Control, no | Blank, no | Enz1 | Enz3 | Enz2 | Enz4 | Ref | Control, no |
| | enzyme | enzyme | | | | | enzyme | enzyme |
| C | Control, no | Blank, no | Enz1 | Enz3 | Enz2 | Enz4 | Ref | Control, no |
| | enzyme | enzyme | | | | | enzyme | enzyme |
| D | Control, no | Ref | Enz2 | Enz4 | Enz1 | Enz3 | Blank, no | Control, no |
| | enzyme | enzyme | | | | | enzyme | enzyme |
| E | Control, no | Ref | Enz2 | Enz4 | Enz1 | Enz3 | Blank, no | Control, no |
| | enzyme | enzyme | | | | | enzyme | enzyme |
| F | Control, no | Ref | Enz2 | Enz4 | Enz1 | Enz3 | Blank, no | Control, no |
| | enzyme | enzyme | | | | | enzyme | enzyme |

Detergent mix+/−bacteria is added to the wells by adding 0.5 mL M. luteus test solution to each well. 10 μl enzyme solution with the prepared concentration is added according to the setup. The plate is allowed to incubate at 30° C. for 1.5 h. After incubation, the solution is removed from the plates by turning the plate upside down on paper towel in a zip-lock bag. The plate is turned and punched two times, then rinsed with 0.75 mL 0.9% NaCl solution. Solution is removed from the plates by turning the plate upside down on paper towel in a zip-lock bag. The plate is turned and punched two times, and the rinsing and punching step is repeated. 0.5 mL crystal violet 0.095% is added to each well. It is allowed to incubate 15 min on the table, then the supernatant is removed from the plates by turning the plate upside down on paper towel in a zip-lock bag and punching the plate hard twice to secure best removal, repeating until drops of unbound dye solution are removed, followed by gentle rinsing with 1 mL 0.9% NaCl. The rinse solution is removed and punched as described earlier. The color in the wells is dissolved with 0.5 mL 96% ethanol, giving a quick shake by hand until the liquid is clear. Absorbance 595 is measured in the CLARIOstar® reader and if it is higher than 3 the samples are diluted in new wells.

The results, measured as follows, are provided in Table 2 below:

$$Y \% attachment inhibition from {A}_{590} = ( 1 - ( {A}_{590 control} / {A}_{590 enzx} ) ) * 100 \% .$$

$${A}_{590} control = {A}_{590} attachment in detergent solution of M . luteus ;$$

$${A}_{590} enzx = {A}_{590} attachment in detergent + enzyme .$$

TABLE 2
Inhibition of attachment of M. luteus

| | | | |
|---|---|---|---|
| | Model A | Model A | 1 × PBS |
| | pH 7.0 Y % | pH 7.8 Y % | pH 6.0 Y % |
| | attachment | attachment | attachment |
| Enzyme | inhibition | inhibition | inhibition |

Day 1

| | | | |
|---|---|---|---|
| SEQ ID NO: 6 (1 ppm) | 46.9 | 43 | 30.5 |
| SEQ ID NO: 30 (10 ppm) | — | −3.5 | 39.9 |

Day 4

| | | | |
|---|---|---|---|
| SEQ ID NO: 6 (1 ppm) | 41.8 | 45.6 | 22.4 |
| SEQ ID NO: 12 (1 ppm) | 40.9 | 17.5 | — |
| SEQ ID NO: 12 (10 ppm) | 40.8 | 33.9 | 72.9 |

Day 14

| | | | |
|---|---|---|---|
| SEQ ID NO: 6 (1 ppm) | — | 52.4 | 43.2 |
| SEQ ID NO: 15 (0.01 ppm) | — | 23.5 | — |
| SEQ ID NO: 15 (0.1 ppm) | — | 51.5 | — |
| SEQ ID NO: 15 (0.5 ppm) | — | 60.1 | — |
| SEQ ID NO: 15 (1 ppm) | — | 65.2 | — |
| SEQ ID NO: 15 (2 ppm) | — | 59.1 | — |
| SEQ ID NO: 15 (5 ppm) | — | 62.6 | 17.5 |

Day 22

| | | | |
|---|---|---|---|
| SEQ ID NO: 6 (1 ppm) | — | 50.7 | — |
| SEQ ID NO: 21 (1 ppm) | — | 5.4 | — |
| SEQ ID NO: 21 (10 ppm) | — | 34.8 | — |

Day 23

| | | | |
|---|---|---|---|
| SEQ ID NO: 6 (1 ppm) | — | 57.1 | — |
| SEQ ID NO: 99 (1 ppm) | — | 42.7 | — |
| SEQ ID NO: 99 (10 ppm) | — | 47.5 | — |

Day 25

| | | | |
|---|---|---|---|
| SEQ ID NO: 6 (1 ppm) | — | 73.9 | — |
| SEQ ID NO: 18 (1 ppm) | — | 67.1 | — |
| SEQ ID NO: 18 (10 ppm) | — | 88.2 | — |

Day 28

| | | | |
|---|---|---|---|
| SEQ ID NO: 6 (1 ppm) | — | 32.5 | — |
| SEQ ID NO: 108 (1 ppm) | — | 13.3 | — |
| SEQ ID NO: 108 (2.5 ppm) | — | 17.1 | — |
| SEQ ID NO: 108 (5 ppm) | — | 21.2 | — |
| SEQ ID NO: 108 (10 ppm) | — | 24.6 | — |

Day 29

| | | | |
|---|---|---|---|
| SEQ ID NO: 6 (1 ppm) | — | 31.8 | — |
| SEQ ID NO: 9 (1 ppm) | — | 17.8 | — |
| SEQ ID NO: 9 (10 ppm) | — | 52 | — |

Day 30

| | | | |
|---|---|---|---|
| SEQ ID NO: 6 (1 ppm) | — | 34.5 | — |
| SEQ ID NO: 27 (1 ppm) | — | 1.3 | — |
| SEQ ID NO: 27 (10 ppm) | — | 4.2 | — |
| SEQ ID NO: 45 (1 ppm) | — | 20.1 | — |
| SEQ ID NO: 45 (10 ppm) | — | 27.3 | — |

Day 31

| | | | |
|---|---|---|---|
| SEQ ID NO: 6 (1 ppm) | — | 30.7 | — |
| SEQ ID NO: 48 (10 ppm) | — | 12 | — |
| SEQ ID NO: 69 (10 ppm) | — | 3.2 | — |

Day 32

| | | | |
|---|---|---|---|
| SEQ ID NO: 6 (1 ppm) | — | 34.4 | — |
| SEQ ID NO: 87 (1 ppm) | — | 4.1 | — |
| SEQ ID NO: 87 (10 ppm) | — | 10 | — |

The test results have a certain day to day variation, due to, e.g., fluctuations in lab temperature and humidity as well as slight variations in day-to-day cell viability, and the attachment inhibition results should therefore be compared for the same day. Our experience with the assay and the enzymes has shown that the day-to-day fluctuations in attachment and inhibition patterns in general give the same pattern in performance between the enzymes when they are repeated another day. Some enzymes work better at pH 6.0 and 7.0 and others perform optimally at pH 7.8, which has been tested for selected enzymes. SEQ ID NO: 6 is used as a reference enzyme to control the assay and measure the day to day variation.

Conclusion: In this experiment, SEQ ID NO: 6 shows robust inhibitory effects of M. luteus attachment at pH 6, pH 7 and pH 7.8. 10 ppm of SEQ ID NO: 30 shows an inhibitory effect on M. luteus on par with the effect of SEQ ID NO: 6 at a concentration of 1 ppm at pH 6, but no performance at pH 7.8. SEQ ID NO: 12 shows good performance at pH 7 compared to the control enzyme SEQ ID NO: 6. The performance of SEQ ID NO: 12 is less prominent at pH 7.8, but there is still a significant effect. SEQ ID NO: 15 can be dosed very low (0.01 ppm) and still give robust anti-attachment benefits at pH 7.8. At 0.5 ppm and 1.0 ppm there is a tendency for the inhibitory effect of SEQ ID NO: 15 to be higher than that of SEQ ID NO: 6 at a pH of 7.8. At pH 6 performance seems to be lower for SEQ ID NO: 15 at 5 ppm compared to SEQ ID NO: 6 at 1 ppm. SEQ ID Nos: 21, 99, 108, 9, 27, 45, 48, 69 and 87 also show inhibitory attachment benefits. SEQ ID NO: 18 gives very high anti-attachment performance on M. luteus at pH 7.8 using 1 ppm and it increases using 10 ppm.

### Example 5: Preparation of Crude Cell Wall Extracts from Micrococcus luteus and OD Drop Activity Assay

### Preparation of Cell Wall Extracts

Cell wall extracts from Micrococcus luteus were prepared following the protocol described by Mukamolova et al., 2006, Molecular Microbiology 59(1): 84-98. Briefly, M. luteus cells grown overnight in 1L LB medium were centrifuged at 10,000 g for 30 minutes, washed with deionized water, resuspended in 200 ml 5% (w/v) SDS and boiled for 20 minutes. Following centrifugation, the pellet was resuspended in 100 ml 4% (w/v) SDS and boiled again for 20 min. The pellet was then thoroughly washed six times with 100 ml hot (65° C.) water to remove the SDS. Finally, it was washed with 10 ml acetone, air dried overnight, weighed and stored at −20° C.

OD Drop Assay Using Crude Cell Wall Extracts from M. luteus

0.6 g of M. luteus cell wall extracts prepared as described above were resuspended in 15 ml of deionized water (stock solution 40 mg/ml) and passed through a syringe needle to disrupt the large flakes. This stock solution was stored at 4° C.

A cell wall extract working solution was prepared from the stock solution at 0.75 mg/ml in 50 mM MES (2-(N-morpholino) ethanesulfonic acid) pH 6 buffer and two model detergents, model O and A (2.67 g/L model O and 0.44 g/L model A, respectively, in tap water). These working solutions were prepared fresh each time when running an OD drop assay.

Next, 150 μL aliquots of the crude cell wall extract working solution were dispensed in the wells of a 96-well microtiter plate (Thermo Scientific, Nunclon Delta Surface, cat #167008) and mixed with 50 μL of a solution containing 80 ppm of a purified enzyme (3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105 or 108) in 50 mM HEPES 100 mM NaCl pH7 buffer, or the HEPES buffer as a control, and incubated at 30° C. with shaking at 600 rpm in an Eppendorf ThermoMixer C. The absorbance of the samples was measured at 600 nm in a SpectraMax M3 instrument at time=0 and after overnight incubation with the enzyme. Enzymes were tested in duplicate. The average of the OD drop measurements (calculated by the OD obtained after overnight incubation minus OD at time 0) are listed in Table 3 below.

TABLE 3
OD drop values (OD after overnight incubation minus OD time 0)

| SEQ ID NO: | MES + HEPES | Model A | Model O |
|---|---|---|---|
| 3 | 0.27 | 0.03 | 0.00 |
| 6 | 0.42 | 0.13 | 0.21 |
| 9 | 0.20 | 0.03 | 0.00 |
| 12 | 0.35 | 0.20 | 0.46 |
| 15 | 0.65 | 0.79 | 0.54 |
| 18 | 0.71 | 0.90 | 0.82 |
| 21 | 0.10 | 0.02 | 0.00 |
| 24 | 0.12 | 0.00 | 0.00 |
| 27 | 0.08 | 0.03 | 0.00 |
| 30 | 0.15 | 0.49 | 0.50 |
| 33 | 0.36 | 0.15 | 0.32 |
| 36 | 0.12 | 0.00 | 0.00 |
| 39 | 0.36 | 0.25 | 0.36 |
| 42 | 0.21 | 0.02 | 0.00 |
| 45 | 0.30 | 0.03 | 0.00 |
| 48 | 0.10 | 0.03 | 0.00 |
| 51 | 0.23 | 0.00 | 0.00 |
| 54 | 0.03 | 0.17 | 0.23 |
| 57 | 0.23 | 0.33 | 0.58 |
| 60 | 0.11 | 0.23 | 0.30 |
| 63 | 0.18 | 0.15 | 0.30 |
| 66 | 0.47 | 0.16 | 0.40 |
| 69 | 0.46 | 0.16 | 0.23 |
| 72 | 0.45 | 0.17 | 0.29 |
| 75 | 0.53 | 0.20 | 0.55 |
| 78 | 0.29 | 0.19 | 0.32 |
| 81 | 0.64 | 0.16 | 0.27 |
| 84 | 0.60 | 0.15 | 0.35 |
| 87 | 0.35 | 0.17 | 0.30 |
| 90 | 0.18 | 0.16 | 0.23 |
| 93 | 0.78 | 0.19 | 0.10 |
| 96 | 0.36 | 0.15 | 0.14 |
| 99 | 0.27 | 0.72 | 0.66 |
| 102 | 0.25 | 0.16 | 0.21 |
| 105 | 0.10 | 0.15 | 0.25 |
| 108 | 0.60 | 0.03 | 0.16 |

The results in Table 3 show that enzymes giving an OD drop can hydrolyze cell wall extracts present in the solution.

### Example 6: Construction of the PGL Domain, Clades and Phylogenetic Trees

The polypeptides of the invention have hydrolase activity and comprise the Amidase_2 domain as well as clusters such as clades. A phylogenetic tree was constructed from polypeptide sequences containing an Amidase_2 domain, as defined in PFAM (PF01510, Pfam version 31.0 Finn (2016). Nucleic Acids Research, Database Issue 44: D279-D285). The phylogenetic tree was constructed from a multiple alignment of mature polypeptide sequences containing at least one Amidase_2 domain. The sequences were aligned using the MUSCLE algorithm version 3.8.31 (Edgar, 2004, Nucleic Acids Research 32(5): 1792-1797), and a tree was constructed using FastTree version 2.1.8 (Price et al., 2010, PloS one 5(3)) and visualized using iTOL (Letunic & Bork, 2007. Bioinformatics 23(1): 127-128).

Analysis of the phylogenetic tree showed that the polypeptides containing an Amidase_2 domain may be separated into distinct sub-clusters. The sub-clusters are defined by one or more short sequence motifs, as well as by containing an Amidase_2 domain as defined in PFAM (PF01510, Pfam version 31.0). We denoted one sub-cluster comprising the motif N[IV]X[AG][GAS]A[AY][LV]L (SEQ ID NO: 111), situated in positions corresponding to positions 324 to 328 in Micromonospora maritima (SEQ ID NO: 6), as the PGL clade. All polypeptide sequences containing an Amidase_2 domain as well as the motif will be denoted as belonging to the PGL clade. Polypeptides included in the clade are SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 42, SEQ ID NO: 45, SEQ ID NO: 48, SEQ ID NO: 51, SEQ ID NO: 54, SEQ ID NO: 60, SEQ ID NO: 63, SEQ ID NO: 66, SEQ ID NO: 69, SEQ ID NO: 75, SEQ ID NO: 87, SEQ ID NO: 96, SEQ ID NO: 99 and SEQ ID NO: 108.

### Example 7: N-Acetylmuramyl-L-Alanine Amidase Assay

### Substrate Synthesis

The organic syntheses of peptidoglycan fragments (1) and (2) was performed in three steps from commercially available methyl 2-acetamido-4,6-O-benzylidene-2-deoxy-α-D-glucopyranoside and the appropriate peptide sequences. Both peptides used (here named S. aureus peptide and M. luteus peptide; see below) were synthesized and provided by TAG Copenhagen A/S. In the structural formulas below, an asterisk (*) denotes D-stereochemistry.

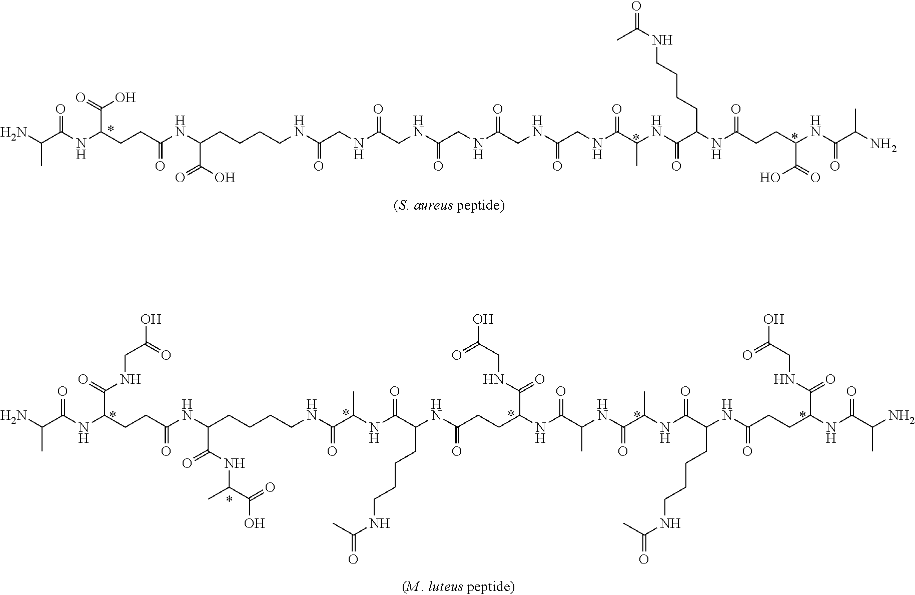 [SMILES: CC(=O)NCCCCC(NC(=O)CCC(NC(=O)C(C)N)C(=O)O)C(=O)NC(C)C(=O)NCC(=O)NCC(=O)NCC(=O)NCC(=O)NCC(=O)NCCCCC(NC(=O)CCC(NC(=O)C(C)N)C(=O)O)C(=O)O ; CC(=O)NCCCCC(NC(=O)CCC(NC(=O)C(C)NC(=O)C(C)NC(=O)C(CCCCNC(C)=O)NC(=O)CCC(NC(=O)C(C)N)C(=O)NCC(=O)O)C(=O)NCC(=O)O)C(=O)NC(C)C(=O)NCCCCC(NC(=O)CCC(NC(=O)C(C)N)C(=O)NCC(=O)O)C(=O)NC(C)C(=O)O]

Synthesis of the peptides modified with muramic acid derivatives was performed, cf. the schematic overview below, by initially coupling methyl 2-acetamido-4,6-O-benzylidene-2-deoxy-α-D-glucopyranoside to (S)-2-chloropropionic acid using the protocol from Hesek et al., J. Org. Chem. 2004, 69, 778-784 to result in compound (3). Then the corresponding muramic acid NHS-ester derivative (4) was synthesized by treating 200 mg of (3) with N-(3-Dimethylaminopropyl)-N-ethylcarbodiimide hydrochloride (EDC-HCl, 1.5 eq.) and N-hydroxysuccinimide (1.15 eq.) in anhydrous dichloromethane (DCM, 2 mL) at room temperature (rt) for 4 hours before the solution was diluted with DCM (10 mL), washed with 2.5% NaHSO₄ and brine, dried over anhydrous NaSO₄, filtered and concentrated in vacuo. The desired product (4) was used without further purification in 2.1 eq. to couple to a peptide (20 mg) in anhydrous dimethylformamide (DMF, 300 μL) at room temperature in the presence of triethylamine (TEA, 3.5 eq.) by overnight reaction. The desired products (S. aureus substrate 1, or M. luteus substrate 2) were used without further purification for amidase assessment.

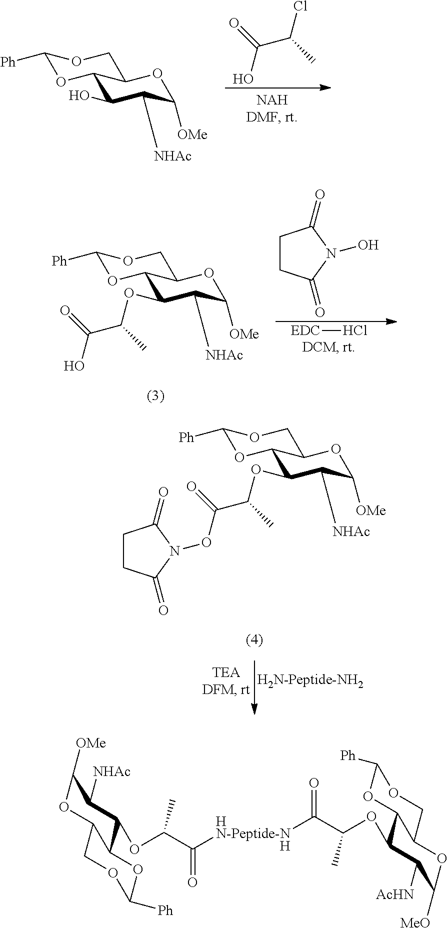

The result is the following modified peptides, S. aureus substrate 1 and M. luteus substrate 2:

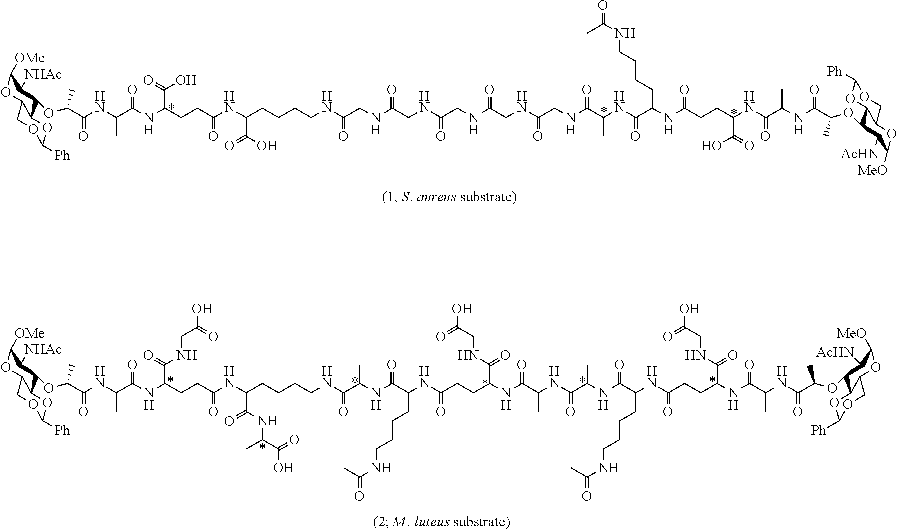

### Amidase Assay

The amidases cleave between the peptide and the muramic acid (MurNAc) motifs to liberate one or two new N-termini, as illustrated in the following:

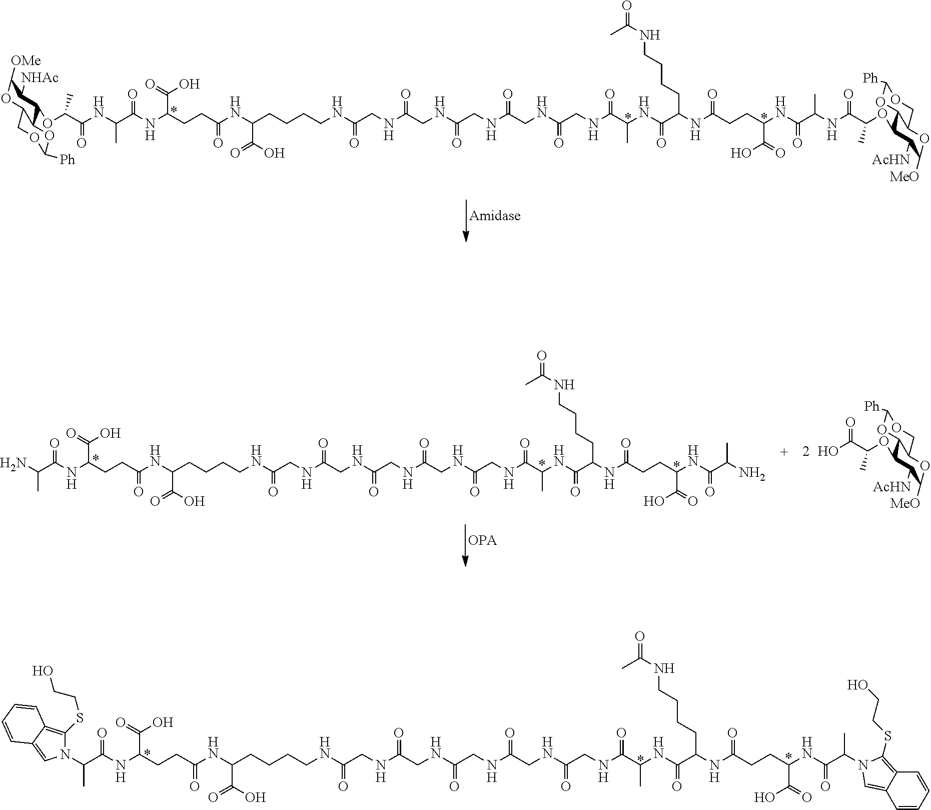

When exposed to standard o-phthaldehyde (OPA) assay conditions, the newly formed peptide amines (N-termini) react to yield a fluorescent readout (excitation=340 nm, emission=455 nm). OPA solution is prepared by dissolving 800 mg o-phthaldehyde in 10 mL 95% EtOH followed by addition of 1 L 0.5 M borate buffer (pH 9.0) containing 2 mL 2-mercaptoethanol.

Amidase reactions were performed by incubating and shaking the amidase (20 pg/mL final conc.) with the MurNAc-peptide-MurNAc substrate (substrate 1 or 2, 5 mM final conc.) in 50 mM MES buffer (pH 6.0, 100 mM NaCl) at 37° C. overnight, before the reaction products were analyzed by OPA assay and MALDI-TOF.

Amidase assessment (OPA assay) was performed by adding 100 μL OPA solution to 10 μL reaction sample. The mixture was transferred to a 96-well plate and monitored in a spectrophotometer (excitation=340 nm, emission=455 nm) after 5 min of incubation. The non-modified S. aureus and M. luteus peptides (i.e., with amino termini as shown above) were included as controls.

### Results

Table 4 below shows the measured fluorescence for the S. aureus and M. luteus peptides (with amino termini) and substrates (with MurNAc termini) alone and after treatment with the amidases of SEQ ID NO: 6 or SEQ ID NO: 33, as well as for the amidases alone (negative control).

It can be seen that the MurNAc-peptide-MurNAc substrates (S. aureus substrate and M. luteus substrate) yield minimal OPA response until treated with the amidase of SEQ ID NO: 6 or SEQ ID NO: 33, where the fluorescent response increases significantly. The amidases appear to have no activity on the non-modified peptides (S. aureus peptide and M. luteus peptide), which was as expected.

MALDI-TOF MS analyses (data not shown) before and after treatment of the S. aureus and M. luteus substrates with the amidase of SEQ ID NO: 6 or SEQ ID NO: 33 confirmed that the OPA response is a result of enzymatic cleavage between the peptide and the MurNAc motifs to liberate the MurNAc motifs, yielding the free peptide N-termini.

TABLE 4

| | Fluorescence |
|---|---|
| Peptide/substrate + enzyme | (RFU; 340, 455 nm) |
| SEQ ID NO: 6 | 29 |
| SEQ ID NO: 33 | 27 |
| S. aureus peptide | 8420 |
| S. aureus peptide + SEQ ID NO: 6 | 8240 |
| S. aureus peptide + SEQ ID NO: 33 | 8547 |
| S. aureus substrate | 515 |
| S. aureus substrate + SEQ ID NO: 6 | 7077 |
| S. aureus substrate + SEQ ID NO: 33 | 5704 |
| M. luteus peptide | 3447 |
| M. luteus peptide + SEQ ID NO: 6 | 3340 |
| M. luteus peptide + SEQ ID NO: 33 | 3298 |
| M. luteus substrate | 381 |
| M. luteus substrate + SEQ ID NO: 6 | 3166 |
| M. luteus substrate + SEQ ID NO: 33 | 1853 |

### Example 8: N-Acetylmuramyl-L-Alanine Amidase Assay, Test of Additional Amidases

Several other enzymes within the same family as the amidase of SEQ ID NO: 6 were tested against the S. aureus substrate (substrate 1) as described in Example 7. This revealed that the amidases of SEQ ID NOs: 3, 45, 24 and 12 had comparable activity towards the S. aureus substrate; see the results in Table 5 below, where the individual enzymes (without peptide or substrate) are included as negative controls.

TABLE 5

| | Fluorescence |
|---|---|
| Peptide/substrate + enzyme | (RFU; 340, 455 nm) |
| S. aureus peptide | 8458 |
| S. aureus substrate | 735 |
| SEQ ID NO: 3 | 33 |
| SEQ ID NO: 6 | 21 |
| SEQ ID NO: 12 | 54 |
| SEQ ID NO: 24 | 108 |
| SEQ ID NO: 45 | 44 |
| S. aureus substrate + SEQ ID NO: 3 | 6810 |
| S. aureus substrate + SEQ ID NO: 6 | 7584 |
| S. aureus substrate + SEQ ID NO: 24 | 6903 |
| S. aureus substrate + SEQ ID NO: 12 | 6602 |
| S. aureus substrate + SEQ ID NO: 45 | 6851 |

The enzyme of SEQ ID NO: 105 was tested in a similar experiment and had comparable activity towards the S. aureus substrate; see Table 6.

TABLE 6

| | Fluorescence |
|---|---|
| Peptide/substrate + enzyme | (RFU; 340, 455 nm) |
| SEQ ID NO: 105 | 37 |
| S. aureus substrate | 756 |
| S. aureus substrate + SEQ ID NO: 105 | 6328 |

1. A cleaning composition comprising a peptidoglycan degradation enzyme, at least one surfactant, and at least one additional cleaning component selected from builders and bleach components.

2. The cleaning composition of claim 1, comprising a peptidoglycan degradation enzyme, at least 5 wt % anionic surfactants, and at least one additional cleaning component selected from at least one builder and at least one bleach component.

3. The cleaning composition according to claim 1, wherein the peptidoglycan degrading enzyme has N-acetylmuramyl-L-alanine amidase activity.

4. The cleaning composition according to claim 1, wherein the peptidoglycan degrading enzyme has peptidoglycan lyase activity.

5. The cleaning composition according to claim 3, wherein the peptidoglycan degradation enzyme has N-acetylmuramyl-L-alanine amidase activity and peptidoglycan lyase activity.

6. The cleaning composition according to claim 1, wherein the peptidoglycan degradation enzyme comprises the motif N[IV]X[AG][GAS]A[AY][LV]L (SEQ ID NO: 111).

7. The cleaning composition of claim 6, wherein the peptidoglycan degradation enzyme is selected from the group consisting of: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 42, SEQ ID NO: 45, SEQ ID NO: 48, SEQ ID NO: 51, SEQ ID NO: 54, SEQ ID NO: 60, SEQ ID NO: 63, SEQ ID NO: 66, SEQ ID NO: 69, SEQ ID NO: 75, SEQ ID NO: 87, SEQ ID NO: 96, SEQ ID NO: 99 and SEQ ID NO: 108, and polypeptides having at least at least 60% sequence identity thereto, and wherein the enzyme has peptidoglycan degradation activity.

8. A cleaning composition according to claim 1, comprising about 5 to about 60 wt % of at least one surfactant, and further comprising:
a. about 5 wt % to about 50 wt % of at least one builder ; and/or
b. about 1 wt % to about 20 wt % of at least one bleach component.

9. A cleaning composition according to claim 1, wherein the polypeptide having peptidoglycan degradation activity is selected from the group consisting of:
i. a polypeptide having at least at least 60% sequence identity to SEQ ID NO: 3,
ii. a polypeptide having at least at least 60% sequence identity to SEQ ID NO: 6,
iii. a polypeptide having at least at least 60% sequence identity to SEQ ID NO: 9,
iv. a polypeptide having at least at least 60% sequence identity to SEQ ID NO: 12,
v. a polypeptide having at least at least 60% sequence identity to SEQ ID NO: 15,
vi. a polypeptide having at least at least 60% sequence identity to SEQ ID NO: 18,
vii. a polypeptide having at least at least 60% sequence identity to SEQ ID NO: 21,
viii. a polypeptide having at least at least 60% sequence identity to SEQ ID NO: 24,
ix. a polypeptide having at least at least 60% sequence identity to SEQ ID NO: 27,
x. a polypeptide having at least at least 60% sequence identity to SEQ ID NO: 30,
xi. a polypeptide having at least at least 60% sequence identity to SEQ ID NO: 33,
xii. a polypeptide having at least at least 60% sequence identity to SEQ ID NO: 36,
xiii. a polypeptide having at least at least 60% sequence identity to SEQ ID NO: 39,
xiv. a polypeptide having at least at least 60% sequence identity to SEQ ID NO: 42,
xv. a polypeptide having at least at least 60% sequence identity to SEQ ID NO: 45,
xvi. a polypeptide having at least at least 60% sequence identity to SEQ ID NO: 48,
xvii. a polypeptide having at least at least 60% sequence identity to SEQ ID NO: 51,
xviii. a polypeptide having at least at least 60% sequence identity to SEQ ID NO: 54,
xix. a polypeptide having at least at least 60% sequence identity to SEQ ID NO: 57,
xx. a polypeptide having at least at least 60% sequence identity to SEQ ID NO: 60,
xxi. a polypeptide having at least at least 60% sequence identity to SEQ ID NO: 63,
xxii. a polypeptide having at least at least 60% sequence identity to SEQ ID NO: 66,
xxiii. a polypeptide having at least at least 60% sequence identity to SEQ ID NO: 69,
xxiv. a polypeptide having at least at least 60% sequence identity to SEQ ID NO: 72,
xxv. a polypeptide having at least at least 60% sequence identity to SEQ ID NO: 75,
xxvi. a polypeptide having at least at least 60% sequence identity to SEQ ID NO: 78,
xxvii. a polypeptide having at least at least 60% sequence identity to SEQ ID NO: 81,
xxviii. a polypeptide having at least at least 60% sequence identity to SEQ ID NO: 84,
xxix. a polypeptide having at least at least 60% sequence identity to SEQ ID NO: 87
xxx. a polypeptide having at least at least 60% sequence identity to SEQ ID NO: 90,
xxxi. a polypeptide having at least at least 60% sequence identity to SEQ ID NO: 93,
xxxii. a polypeptide having at least at least 60% sequence identity to SEQ ID NO: 96,
xxxiii. a polypeptide having at least at least 60% sequence identity to SEQ ID NO: 99,
xxxiv. a polypeptide having at least at least 60% sequence identity to SEQ ID NO: 102,
xxxv. a polypeptide having at least at least 60% sequence identity to SEQ ID NO: 105, and
xxxvi. a polypeptide having at least at least 60% sequence identity to SEQ ID NO: 108.

10. The cleaning composition according to claim 9, wherein the polypeptide has N-acetylmuramyl-L-alanine amidase and peptidoglycan lyase activity.

11-12. (canceled)

13. A method of cleaning on an item, comprising
a) contacting the item with a solution comprising a peptidoglycan degradation enzyme having peptidoglycan lyase activity and N-acetylmuramyl-L-alanine amidase activity, and a cleaning component, wherein the cleaning component is selected from 5 to 60 wt % of at least one surfactant; 5 to 50 wt % of at least one builder; and 1 to 20 wt % of at least one bleach component, and optionally
b) rinsing the item;
wherein the item is a textile.


SEQUENCE LISTING (111 sequences)

SEQ ID NO: 1 - DNA, 1380 bp, Hamadaea tsunoensis
  sig_peptide 1..87
  mat_peptide 88..1380
  source 1..1380: mol_type = genomic DNA
  CDS 1..1380
atgacagtaccacctacacgtcgtgctactttcatgttaggagcgttgatcttagccgctggtgtagctgcaccgccagcaagcgctgctgctgctccagtagcgcctgacgcattaacagtagcgtcaactccagcatcaactggtagccttactgctgcttttgatgctgcagcaacacgttatggagttccacgtgacttattgatcgctattggttacgctgaaacacaccttgatggacacgcaggcactccatctgcagctggtggatatggtgttatgaatcttactggcaatccggctgttcacactcttgctgaagcttctcgcttaactggccttaaagctagcacgttagagaataaccaagctgctaacatccttggtgcggctgcagttttacgctcttacgctgctgatttgaaaactgctcaacgtgactctgtagacaattggtacgctgctgtagctcgttatggtggcgcaactgatccgtctgttgctcgcctttatgctgatacagtttacgatcttcttgcgacaggtttcggagttccagctaaaggcgttagcgttacagctcgtgctgtagcacctcaacgtggtactcttgctactgcgcgtgcatcattggactctgcagactacggtcctgctgcttgggctccagcatctacatctaactacacagttgcaaaccgcgaaacagactataacatcaattacattgttatccacgtaactcaaggatcttacgctggctctatttcttggttccaaaatcctgcggctcaggtttctgctcattacgttgtacgttcatctgatggtgccatcacgcagtctgttcgtgaaaaagatatcgcttggcacgcaggcaactggacatacaacacgcaagctatcggcatcgaacatgaaggctatatcgacgacccttcatggttcactgacgcaatgtatcgttcttcagcagctcttacacgttcacttacaacgaaatacgctattcctcgtgaccgcagccacatcatcggtcatatcgaagttccttctgcaacgcacacggatcctggtcagtattggaactggacttactacatgcaattggtaaacggagtaacgggtatcggaacaggaacggtaaacgtatctggctcattgaacattcgctcaggtcctggtacaggctacgctgttgctggatctcttgcgaacggagcgggagtttctgtttactgccaagctacgggcacgacagtaacgggtacttacggcactagcaacatctgggaccgtatcggtacgaataagtatgttgcagatgcgtatgtattgactggttctgacggctttatccctggtgtaccacgttgc

SEQ ID NO: 2 - protein, 460 aa, Hamadaea tsunoensis
MTVPPTRRATFMLGALILAAGVAAPPASAAAAPVAPDALTVASTPASTGSLTAAFDAAATRYGVPRDLLIAIGYAETHLDGHAGTPSAAGGYGVMNLTGNPAVHTLAEASRLTGLKASTLENNQAANILGAAAVLRSYAADLKTAQRDSVDNWYAAVARYGGATDPSVARLYADTVYDLLATGFGVPAKGVSVTARAVAPQRGTLATARASLDSADYGPAAWAPASTSNYTVANRETDYNINYIVIHVTQGSYAGSISWFQNPAAQVSAHYVVRSSDGAITQSVREKDIAWHAGNWTYNTQAIGIEHEGYIDDPSWFTDAMYRSSAALTRSLTTKYAIPRDRSHIIGHIEVPSATHTDPGQYWNWTYYMQLVNGVTGIGTGTVNVSGSLNIRSGPGTGYAVAGSLANGAGVSVYCQATGTTVTGTYGTSNIWDRIGTNKYVADAYVLTGSDGFIPGVPRC

SEQ ID NO: 3 - protein, 431 aa, Hamadaea tsunoensis
AAAPVAPDALTVASTPASTGSLTAAFDAAATRYGVPRDLLIAIGYAETHLDGHAGTPSAAGGYGVMNLTGNPAVHTLAEASRLTGLKASTLENNQAANILGAAAVLRSYAADLKTAQRDSVDNWYAAVARYGGATDPSVARLYADTVYDLLATGFGVPAKGVSVTARAVAPQRGTLATARASLDSADYGPAAWAPASTSNYTVANRETDYNINYIVIHVTQGSYAGSISWFQNPAAQVSAHYVVRSSDGAITQSVREKDIAWHAGNWTYNTQAIGIEHEGYIDDPSWFTDAMYRSSAALTRSLTTKYAIPRDRSHIIGHIEVPSATHTDPGQYWNWTYYMQLVNGVTGIGTGTVNVSGSLNIRSGPGTGYAVAGSLANGAGVSVYCQATGTTVTGTYGTSNIWDRIGTNKYVADAYVLTGSDGFIPGVPRC

SEQ ID NO: 4 - DNA, 1548 bp, Micromonospora maritima
  sig_peptide 1..90
  mat_peptide 91..1545
  source 1..1548: mol_type = genomic DNA
  CDS 1..1545
gtgacgattcggagaccctcgcgtcgggtgagtctgctcggcggcgccatgatcctcatgatcggcctgaccggccagccggcccaggccgcgcctgcacaccgcgcgcagcctctcgccgcggccttcgcgcaggccgcggccgattccgacgtgccgcgcgacctgctcgccgcgctcgggtacgccgagacccgcctggacggccacggcggcgcgcccagcgcctccggcgggtacggcgtgatgcacctgaccagcaacccgaaggtgcggacgctcgacgaggccgcgcgccggacccggctggaccgcgccgagctgcgtacccgggacgcggcgaacgtggccggcgcggcggcggtgctgcgttcctacgccgacgaggccgggctcagcgcggcgcagcgcgacgacgtcaaccagtggtacggcccgatcgcccgctacggcggcgcgaccgacggggccaccgcccggctgtacgccgactccgtgtacgacctgctcgcccggggcttcatcgcgaccacggccggcggcgaggtcagcgtggacggccgtccggtcgcaccgcagcggggccggtacgccgacgtggcgccgctgggcaccggtgacttcggcaccctgagcaccgactacggcccggcggcctgggtgccggccaactcgtccaactacacggtctccagccgcgagtcggcgtacccgatcaactacatcgtcatccacaccatgcagggcagctacgccggctcgatcagctggttccagaacgccgccgccggcaccagcgcgcactacctgctccgctcctccgacggtgcggtgacccagatggtgcgggacaaggacatcgcctggcacgccggcaactggacctacaacacccagtcgatcggcatcgagcacgaggggtacgtcgacaacgcctcctggtacaccgacgcgatgtaccggtcgtcggcggcgctgacccggtacctgtgcgacaagtacggcatcccgaagacccgcaccaacatcatcgggcacaaccaggtgccgggcgccacgcacaccgacccgggtccgaactggaactggacctactacatgcagctcgtcaccggcggcaccacgcccccgccgaccgactggtcgacgatcgtggacaacaccaccgccggccggttcaccgcgagcgccaactggggcacctcgacgtactcggcgcagcgctacggcgccgactaccggtacgccaaccccgtcgcggccagcgacaccgcctggtacaaggtgaacatcccggcgaccgccacctaccgggtggaggtctggtatccggccgtggccggctacaacacctccacgccgtacatcgtggcgacgaccagcggcaaccagacggtctcggtgaaccagacggcgaacggcggcgggtggcggtcgctgggcaccttcaccctggccgccggggacgccaacaaggtgggtgtcagcaggtggtccggcagcaccgggtacgtgatcgccgacgccatccgcgtcacccgcgtctag

SEQ ID NO: 5 - protein, 515 aa, Micromonospora maritima
VTIRRPSRRVSLLGGAMILMIGLTGQPAQAAPAHRAQPLAAAFAQAAADSDVPRDLLAALGYAETRLDGHGGAPSASGGYGVMHLTSNPKVRTLDEAARRTRLDRAELRTRDAANVAGAAAVLRSYADEAGLSAAQRDDVNQWYGPIARYGGATDGATARLYADSVYDLLARGFIATTAGGEVSVDGRPVAPQRGRYADVAPLGTGDFGTLSTDYGPAAWVPANSSNYTVSSRESAYPINYIVIHTMQGSYAGSISWFQNAAAGTSAHYLLRSSDGAVTQMVRDKDIAWHAGNWTYNTQSIGIEHEGYVDNASWYTDAMYRSSAALTRYLCDKYGIPKTRTNIIGHNQVPGATHTDPGPNWNWTYYMQLVTGGTTPPPTDWSTIVDNTTAGRFTASANWGTSTYSAQRYGADYRYANPVAASDTAWYKVNIPATATYRVEVWYPAVAGYNTSTPYIVATTSGNQTVSVNQTANGGGWRSLGTFTLAAGDANKVGVSRWSGSTGYVIADAIRVTRV

SEQ ID NO: 6 - protein, 485 aa, Micromonospora maritima
APAHRAQPLAAAFAQAAADSDVPRDLLAALGYAETRLDGHGGAPSASGGYGVMHLTSNPKVRTLDEAARRTRLDRAELRTRDAANVAGAAAVLRSYADEAGLSAAQRDDVNQWYGPIARYGGATDGATARLYADSVYDLLARGFIATTAGGEVSVDGRPVAPQRGRYADVAPLGTGDFGTLSTDYGPAAWVPANSSNYTVSSRESAYPINYIVIHTMQGSYAGSISWFQNAAAGTSAHYLLRSSDGAVTQMVRDKDIAWHAGNWTYNTQSIGIEHEGYVDNASWYTDAMYRSSAALTRYLCDKYGIPKTRTNIIGHNQVPGATHTDPGPNWNWTYYMQLVTGGTTPPPTDWSTIVDNTTAGRFTASANWGTSTYSAQRYGADYRYANPVAASDTAWYKVNIPATATYRVEVWYPAVAGYNTSTPYIVATTSGNQTVSVNQTANGGGWRSLGTFTLAAGDANKVGVSRWSGSTGYVIADAIRVTRV

SEQ ID NO: 7 - DNA, 1527 bp, Paenibacillus sp.
  sig_peptide 1..78
  mat_peptide 79..1527
  source 1..1527: mol_type = genomic DNA
  CDS 1..1527
atgggaattcgtcgcgtttctcgccttcttagcatctctctttctgccatgcttcttcttccatttacagttccagctgcatcatttgcagcagacgacgctgcggtatcagcgaactctgcatctgcaggaaaaggttctcttcaaaaagctttcgaagccgctagccaagagtttggcgttccagtagagatccttcttggccttagctacgctgagactcgctggaacgaccacgagggaaagccttctcaacttaacggatatggccttatgcaccttgctgagaacccgaaaaactcttcacttagcacagctgctgagcaacttaaagtagacaaacaacttcttaagacagacaaagcagttaacattcgcggatctgcggctgtacttgcaggccttgcaaaggcgaaaaacaacggaaaacttccggcttctcttgctgactggtatacgacagtagcagcgtattctggaatcgacgaccttccgcttgctcgcgtttacgctgacgaggttttcaaagttatcaacgagggaaaacaagcgcttagcggcacagagatccttcaccttaaccctacgccagttactccgaatcgcgctgagtacacgcaagctactcttgctgcgactgctatggactaccctggtgctatctggaacgctgcttactctggcaactactctgtaggctctcgtggcccaggagacatctctaacatcgtaatccatactacacaaggttcatatgctggcacaatcaactggttcaaagaccctgctgctgtagtttcagcgcattatgttgttcgcagctctgacggccagatcacacaaatggtacgcgacaaagacatcgcataccatgctcgctctgcaaacagcacatcacttggcatcgagcatgagggctatgtaactgaccctgcatggtatactgactcaatgtatcgctcatctgcagctcttactcgctggctttgcgaccagtatggcatcccaaaaacacgcacagctatcaaaggccattctgagatgcctggtaacgaccacacagaccctggcagcaactgggactggacttactacatgtcacttgttaaccctccagtaactggtggcatcatcgtagacaacgcaacagctggcgctttcacggcgagcgcaaactggggtacagctacttggaacactgagaagtatggctctgactatcgctacacaacacctcaagctgtttcagacccagcatggttccaagcgacaatccctacagctggctcttatgacgtttatgcttggtggccttcaaacgctgcgtataacgacaaaacgccgtttatcatcagcacatctactggtaaccaaacagttaacgttaaccagcaagctaacggtggtaaatggatgcttcttggtaagtacactcttaacagcggcacgtataacgtagttggcatctctcgctggacatcaggaactggcaacatcttcgccgacgctatccgccttgtaatcaaa

SEQ ID NO: 8 - protein, 509 aa, Paenibacillus sp.
MGIRRVSRLLSISLSAMLLLPFTVPAASFAADDAAVSANSASAGKGSLQKAFEAASQEFGVPVEILLGLSYAETRWNDHEGKPSQLNGYGLMHLAENPKNSSLSTAAEQLKVDKQLLKTDKAVNIRGSAAVLAGLAKAKNNGKLPASLADWYTTVAAYSGIDDLPLARVYADEVFKVINEGKQALSGTEILHLNPTPVTPNRAEYTQATLAATAMDYPGAIWNAAYSGNYSVGSRGPGDISNIVIHTTQGSYAGTINWFKDPAAVVSAHYVVRSSDGQITQMVRDKDIAYHARSANSTSLGIEHEGYVTDPAWYTDSMYRSSAALTRWLCDQYGIPKTRTAIKGHSEMPGNDHTDPGSNWDWTYYMSLVNPPVTGGIIVDNATAGAFTASANWGTATWNTEKYGSDYRYTTPQAVSDPAWFQATIPTAGSYDVYAWWPSNAAYNDKTPFIISTSTGNQTVNVNQQANGGKWMLLGKYTLNSGTYNVVGISRWTSGTGNIFADAIRLVIK

SEQ ID NO: 9 - protein, 483 aa, Paenibacillus sp.
ASFAADDAAVSANSASAGKGSLQKAFEAASQEFGVPVEILLGLSYAETRWNDHEGKPSQLNGYGLMHLAENPKNSSLSTAAEQLKVDKQLLKTDKAVNIRGSAAVLAGLAKAKNNGKLPASLADWYTTVAAYSGIDDLPLARVYADEVFKVINEGKQALSGTEILHLNPTPVTPNRAEYTQATLAATAMDYPGAIWNAAYSGNYSVGSRGPGDISNIVIHTTQGSYAGTINWFKDPAAVVSAHYVVRSSDGQITQMVRDKDIAYHARSANSTSLGIEHEGYVTDPAWYTDSMYRSSAALTRWLCDQYGIPKTRTAIKGHSEMPGNDHTDPGSNWDWTYYMSLVNPPVTGGIIVDNATAGAFTASANWGTATWNTEKYGSDYRYTTPQAVSDPAWFQATIPTAGSYDVYAWWPSNAAYNDKTPFIISTSTGNQTVNVNQQANGGKWMLLGKYTLNSGTYNVVGISRWTSGTGNIFADAIRLVIK

SEQ ID NO: 10 - DNA, 1479 bp, Nonomuraea sp.
  sig_peptide 1..66
  mat_peptide 67..1479
  source 1..1479: mol_type = genomic DNA
  CDS 1..1479
atgagccgtcttgcagcaatcgtttttgctgtacttcttgctttcggtttttctcctgcgtacgcagcggcagacccacttacagaagctttcgaccgtgctgcggctgctcacgacgtacctcgtgaccttcttgttgctttggcttacgctgaaacacacttaaacggccacaacggcgaaccatctgcatcaggtggctatggaatgatgcaccttgtttctaatccaacaacaaaagctcttgctaaagctgccgaacttacaggattacctgcagctgagttacgtgctgacgatgcagcgaacatcttaggtggtgcagcagtacttcgtagccatgctgatgctcttggtttggacgaagcagcacgtaaagatgctggccgttggtaccaagcagttgcggaatacggtaacgcatctacaccagagactgcacgtctttatgcggatgcagtatacgaattccttggcaaaggcttcgaggctgcaggtgttaaagtagctccacaagaagtaactgccgaccgtggtgcgtacgctaagacacgcgagttaacagctgcggctagcccagactaccctgacggcacatgggttgctgcttcttcttctaactacactgcatcttctcgcccatcaagctacgcgatcgatcgtgttgtaatccacgtaactcagggatcatatgctggcagcatcagctggtttcaaaacccttctgctggcgtttctgcacactacgttattcgttcttctgatggtgctgttactcaaatggttcgtaacaaagatgttgcttggcatgcaggcaactggggctataacacacgttctatcggcattgaacatgaaggatgggtatcagatgcttcttggttcactgaagctatgtatcgtagctctggtgcattgactcgttacatttgtgacaaatacggcatccctaaagatcgttcacacatcattggtcacaaccaagtaccaggagcgactcatacagacccaggtagccattgggattggacgaagtatatgtcttatgttaatggtggaggaggaacaccatcttggtctgttactgtagacaatactacagcaggcaaattcacagcttcagcaaactggggtacttctgcctattctggccaacgccatggtgctgactaccgtttcgcaactcctcttgcagcatctgatcctgcttggttcaaagctaacatcccttctgcaggttcttatcgtgttgaagtttggtatccgtctgaccctggctataactcttcagctccttacatcgtagctgcttctggcggtaaccaaacagttttcgttgatcaacgttcaggaggcggtggatggcgcactcttggaacgttctctttgacggctggcgaacacgacgttgtaggagttagccgctggacatctggcacgggctatgtagtagcagatgctgtacgcatttcacattta

SEQ ID NO: 11 - protein, 493 aa, Nonomuraea sp.
MSRLAAIVFAVLLAFGFSPAYAAADPLTEAFDRAAAAHDVPRDLLVALAYAETHLNGHNGEPSASGGYGMMHLVSNPTTKALAKAAELTGLPAAELRADDAANILGGAAVLRSHADALGLDEAARKDAGRWYQAVAEYGNASTPETARLYADAVYEFLGKGFEAAGVKVAPQEVTADRGAYAKTRELTAAASPDYPDGTWVAASSSNYTASSRPSSYAIDRVVIHVTQGSYAGSISWFQNPSAGVSAHYVIRSSDGAVTQMVRNKDVAWHAGNWGYNTRSIGIEHEGWVSDASWFTEAMYRSSGALTRYICDKYGIPKDRSHIIGHNQVPGATHTDPGSHWDWTKYMSYVNGGGGTPSWSVTVDNTTAGKFTASANWGTSAYSGQRHGADYRFATPLAASDPAWFKANIPSAGSYRVEVWYPSDPGYNSSAPYIVAASGGNQTVFVDQRSGGGGWRTLGTFSLTAGEHDVVGVSRWTSGTGYVVADAVRISHL

SEQ ID NO: 12 - protein, 471 aa, Nonomuraea sp.
AADPLTEAFDRAAAAHDVPRDLLVALAYAETHLNGHNGEPSASGGYGMMHLVSNPTTKALAKAAELTGLPAAELRADDAANILGGAAVLRSHADALGLDEAARKDAGRWYQAVAEYGNASTPETARLYADAVYEFLGKGFEAAGVKVAPQEVTADRGAYAKTRELTAAASPDYPDGTWVAASSSNYTASSRPSSYAIDRVVIHVTQGSYAGSISWFQNPSAGVSAHYVIRSSDGAVTQMVRNKDVAWHAGNWGYNTRSIGIEHEGWVSDASWFTEAMYRSSGALTRYICDKYGIPKDRSHIIGHNQVPGATHTDPGSHWDWTKYMSYVNGGGGTPSWSVTVDNTTAGKFTASANWGTSAYSGQRHGADYRFATPLAASDPAWFKANIPSAGSYRVEVWYPSDPGYNSSAPYIVAASGGNQTVFVDQRSGGGGWRTLGTFSLTAGEHDVVGVSRWTSGTGYVVADAVRISHL

SEQ ID NO: 13 - DNA, 1920 bp, Lysobacter antibioticus
  sig_peptide 1..117
  mat_peptide 118..1917
  source 1..1920: mol_type = genomic DNA
  CDS 1..1917
atgaacgaatattccattgcgcgccatggggccggtaccggcgtgcgttcgctgtcgtggtcgctgaccctcgccctgctggcgctcgccgcgccgtgggccgcgcaggcccaggccgcacccgaagaccgcgccctggcccagcgcctgcagatcgaggaatcgctgcaacgcgtcgaccgcgcgctgtacgcggactacttccgccaggcctatgcgcgttacccgtcgatcccggccggcaccctggaatcgatcgcctacgtgatgagccgctggcagcaactgcagcccggcccggccgcgggctatggcgaacagcaccagcacatgccgcgctcgtacggcgtcatgggcctgtaccacggcgagggtttcgccgatcaagtcagcgaaggcgcgcgcctgatcggcgtgcccgcggcgcgcgtgcagcgcgatccgctcagcaacatcctcgcctcggcggccttgctcgatcgcgagctgcgcgccgacggggtcggtgccaagtcggcgatcgaagccacgcgtccggcgctggagcgctacgccggtttcgccggcaatgcgggcaagagcgcgatccaggatcacgcccgttccagtttcgccttcgacgtgctgctggcgcaggacaagggcgtcaacgaccgcggcatcgtcgtgcccacgcgcgcggtcgcctgggaacgcgccttcgatgcgcgcaagctggtgcagctgcgcgcgccgttcgtgcgtctggacgtgagccgcgatcgggtcgaggccggcgcgttgagggacgacggcgcgttcgcgatcgatccgctcagcgaaaccctgcgcgcgccatcgctgaccgcggccgacgaaaagagcaccgattacggcccggcgctgtgggtcgcttcgccttaccactccacccgcacgtcgtacgactcggtgaccatccacacgatgcagggctattacgccggcagcatctcgtggttccagaacaaccccaacagcgtcagcgcgcattacctgatccgcagctccgacggccagatcacccagatggtgcgcgagaaccgcgcggcccatcacgtcggcgtgcataacaagaccacgctcggcatcgagcacgaaggcttcatcaacaacgcgagttggtacaccgcggcgatgtacaacgcctcggcggcgttgacccggcacttctgcgcgacctacagcgcgatcagctgcgcgagcgcgttcaagggcccggccggcagcggcatcaacgtgttgccggccagcgtcaaggtcaagggccaccagcactacagcagccagacccacaccgatccgggcatcaactgggattgggcgcgctactacaacctgctcaatccgggcaatccgcccggcggcggcagcgtgatcgacagtttcgaaagcacggtcgggcatttcgacaccggcccggcgtattcgggcagcaccaccggcatcgccgcgacctcgctgagcgaacgcaactgcaccacgcgcaagaacggcgagtgctcgctgcggctgctgctgaaagacgatgccgccagcgccgatgcctgggcggtgcggctgttgtcgggcagcggcaatccgggcagcaacgcggccctgacgcgcgccaacggcaaggtcggtttctgggtcttcaccggcgcgaccgggatgagcgcggcgatcggcatcgacgacagcgacggcaccgagcgctcggtgagccgcgcgattgcggccaacacctggacctatctggagtggagcctgaccgacgacgcgcagtgggatgcgtgggtcggcggcgccaacggcgcgatcaccgccgcgtcggtgaagctcgatgcggtgtggttctaccgcgatcagacctcgttcgacgtgaacgtgtacgtcgacgatgtgcaggtgaagaactga

SEQ ID NO: 14 - protein, 639 aa, Lysobacter antibioticus
MNEYSIARHGAGTGVRSLSWSLTLALLALAAPWAAQAQAAPEDRALAQRLQIEESLQRVDRALYADYFRQAYARYPSIPAGTLESIAYVMSRWQQLQPGPAAGYGEQHQHMPRSYGVMGLYHGEGFADQVSEGARLIGVPAARVQRDPLSNILASAALLDRELRADGVGAKSAIEATRPALERYAGFAGNAGKSAIQDHARSSFAFDVLLAQDKGVNDRGIVVPTRAVAWERAFDARKLVQLRAPFVRLDVSRDRVEAGALRDDGAFAIDPLSETLRAPSLTAADEKSTDYGPALWVASPYHSTRTSYDSVTIHTMQGYYAGSISWFQNNPNSVSAHYLIRSSDGQITQMVRENRAAHHVGVHNKTTLGIEHEGFINNASWYTAAMYNASAALTRHFCATYSAISCASAFKGPAGSGINVLPASVKVKGHQHYSSQTHTDPGINWDWARYYNLLNPGNPPGGGSVIDSFESTVGHFDTGPAYSGSTTGIAATSLSERNCTTRKNGECSLRLLLKDDAASADAWAVRLLSGSGNPGSNAALTRANGKVGFWVFTGATGMSAAIGIDDSDGTERSVSRAIAANTWTYLEWSLTDDAQWDAWVGGANGAITAASVKLDAVWFYRDQTSFDVNVYVDDVQVKN

SEQ ID NO: 15 - protein, 600 aa, Lysobacter antibioticus
APEDRALAQRLQIEESLQRVDRALYADYFRQAYARYPSIPAGTLESIAYVMSRWQQLQPGPAAGYGEQHQHMPRSYGVMGLYHGEGFADQVSEGARLIGVPAARVQRDPLSNILASAALLDRELRADGVGAKSAIEATRPALERYAGFAGNAGKSAIQDHARSSFAFDVLLAQDKGVNDRGIVVPTRAVAWERAFDARKLVQLRAPFVRLDVSRDRVEAGALRDDGAFAIDPLSETLRAPSLTAADEKSTDYGPALWVASPYHSTRTSYDSVTIHTMQGYYAGSISWFQNNPNSVSAHYLIRSSDGQITQMVRENRAAHHVGVHNKTTLGIEHEGFINNASWYTAAMYNASAALTRHFCATYSAISCASAFKGPAGSGINVLPASVKVKGHQHYSSQTHTDPGINWDWARYYNLLNPGNPPGGGSVIDSFESTVGHFDTGPAYSGSTTGIAATSLSERNCTTRKNGECSLRLLLKDDAASADAWAVRLLSGSGNPGSNAALTRANGKVGFWVFTGATGMSAAIGIDDSDGTERSVSRAIAANTWTYLEWSLTDDAQWDAWVGGANGAITAASVKLDAVWFYRDQTSFDVNVYVDDVQVKN

SEQ ID NO: 16 - DNA, 1548 bp, Micromonospora sp.
  sig_peptide 1..93
  mat_peptide 94..1545
  source 1..1548: mol_type = genomic DNA
  CDS 1..1545
gtgactgttcggagaccctcgcgtcgggtgagccaactgctcggcggcgcagcgattttgatgatcggcctgaccagccagccggcccaggccgcgccgcagcagggcgcggagacgctcgccgccgccttcgaccaggcggcggcccgctccgacgtgccgcgcgacctgctcgccgcgctcgggtacgcggagacccggctggacggtcacaacggcgagcccagcgcctccggcgggtacggcgtgatgcacctgaccagcaacccgaaggtgcggaccctcgacgaggccgcgcgccgtgcccgactggaccgcaccgaactgcgcacccgtgacgcggcgaacgtcgccggcgcggcggcggtgctccgctcgtacgccgatcaggccgggctcaccgcgaagcagcgcgacgacgtcaaccagtggtacggcttgatcgcccgctacggcggttcgtcggacaaggccaccgcccggctgtacgccgacgccgtgtacgacctgctcggcagcggcttcagggcgaccacggccaccggcgaggtcaccgtggacggccgtccggtcgcgccccagcggggcgactacgccgcggtggcgccgctgggcgccgccgacatgggcacccagagcaccgactacggcccggcggcctgggtggcggcgaactcgtccaactacacggcctccagccgcgagtcgtcgtacccgatcaactacatcatcatccacaccatgcagggcagctacgccggctcgatcagctggttccagaacgcagcggccggcaccagcgcgcactacctgctccgttcctcggacggcgcggtgacccagatggtgcgggacaaggacgtcgcctggcacgccggcaactggacctacaacacccagtcgatcggtatcgagcacgagggctacgtcgacaacgcctcctggtacaccgacgcgatgtaccggtcgtcggccgcgctgacgcggcacctggccgacaagtacggcatcccgaagacccgcagcaacatcatcggtcacaaccaggtgccgggcgccacgcacaccgacccgggtccgaactggaactggacctactacatgcagctcgtcaccggcaccacgacgccgccgccgacctggtcgaccatcgtggacaacaccaccgccggccggttcaccgcgagcgccaactggagcacctcgtcgtactcgtcgcagcgctacggggccgactaccgctacgccaaccctgtcgcggccagcgacgccgcctggtacaaggtgaacatcccggccaccgccacctaccgggtggaggtctggtacccggccgtcgccggttacaacgccaccacgccgtacatcgtggcgaccagcagcggcaaccagacggtcaacgtgaaccagtcggccaacggtggtggttggcgctcgctgggcaacttcaccctggccgccggggacgccaacaaggtgggcatcagccgttggtccggtagcaccggctacgtgatcgccgacgcgatccgcatcacccgcgtctaa

SEQ ID NO: 17 - protein, 515 aa, Micromonospora sp.
VTVRRPSRRVSQLLGGAAILMIGLTSQPAQAAPQQGAETLAAAFDQAAARSDVPRDLLAALGYAETRLDGHNGEPSASGGYGVMHLTSNPKVRTLDEAARRARLDRTELRTRDAANVAGAAAVLRSYADQAGLTAKQRDDVNQWYGLIARYGGSSDKATARLYADAVYDLLGSGFRATTATGEVTVDGRPVAPQRGDYAAVAPLGAADMGTQSTDYGPAAWVAANSSNYTASSRESSYPINYIIIHTMQGSYAGSISWFQNAAAGTSAHYLLRSSDGAVTQMVRDKDVAWHAGNWTYNTQSIGIEHEGYVDNASWYTDAMYRSSAALTRHLADKYGIPKTRSNIIGHNQVPGATHTDPGPNWNWTYYMQLVTGTTTPPPTWSTIVDNTTAGRFTASANWSTSSYSSQRYGADYRYANPVAASDAAWYKVNIPATATYRVEVWYPAVAGYNATTPYIVATSSGNQTVNVNQSANGGGWRSLGNFTLAAGDANKVGISRWSGSTGYVIADAIRITRV

SEQ ID NO: 18 - protein, 484 aa, Micromonospora sp.
APQQGAETLAAAFDQAAARSDVPRDLLAALGYAETRLDGHNGEPSASGGYGVMHLTSNPKVRTLDEAARRARLDRTELRTRDAANVAGAAAVLRSYADQAGLTAKQRDDVNQWYGLIARYGGSSDKATARLYADAVYDLLGSGFRATTATGEVTVDGRPVAPQRGDYAAVAPLGAADMGTQSTDYGPAAWVAANSSNYTASSRESSYPINYIIIHTMQGSYAGSISWFQNAAAGTSAHYLLRSSDGAVTQMVRDKDVAWHAGNWTYNTQSIGIEHEGYVDNASWYTDAMYRSSAALTRHLADKYGIPKTRSNIIGHNQVPGATHTDPGPNWNWTYYMQLVTGTTTPPPTWSTIVDNTTAGRFTASANWSTSSYSSQRYGADYRYANPVAASDAAWYKVNIPATATYRVEVWYPAVAGYNATTPYIVATSSGNQTVNVNQSANGGGWRSLGNFTLAAGDANKVGISRWSGSTGYVIADAIRITRV

SEQ ID NO: 19 - DNA, 1533 bp, Nonomuraea coxensis
  sig_peptide 1..90
  mat_peptide 91..1530
  source 1..1533: mol_type = genomic DNA
  CDS 1..1530
atggaactcgcccgcacgcgattaacagccctcgccacctccttcctcgccgtcctcgtcctcgtcgccacgggccgccccgccctggccgcgcccacggccgcggacggcaccctcaccgccgccttcgccaaggcggcggccgcgtacgacgtgccccgcgacctgctggtcgccctcggctacgccgagacccacctcgacggtcacgacggcaagcccagcgccagcggcggctacggcgtcatgcacctggtcagcaaccccaccaaccactccctggagaaggccgccgagctcaccggccgctccaccggcgaactgcgcgccgacgacgccgccaacgtgctcggcggcgccgccgtactccgctcccacgccgacgccctcggcctcgacgaaacggccaggaaggacgccggacgctggtaccaggccgtcgccaagtacggcaacgccacctcgcccgagctcgcccgcctctacgccgacgccgtctacgagagcctcggcctcggcatcgacatccgcggcgtccaggtcaagccccaggaggtcaccgccgaccgcggcgagtacgccaaggcccgcgacctcaacgccaaggccgacgccggcgtcctcagcaccgactacggccccgccgcctgggtcgccgccagctccagcaactacaccgcctccagccgcccgtcgagctacgccatcgaccgcgtgatcatccacgtgacgcagggctcgtacgccggcaccatctcctggttccagaacccctccgcccaggtctcggcccactacgtggtcaagtcctccaacggcgccatcacccagatggtgcgcgagaaggacgtcgcctggcacgccggcaactggacctacaacacccggtcgatcggcatcgagcacgagggctacgtcaacgacgcctcgtggttcaccgacgcgatgtaccgggcgtccgcggccctcaccaagaacatctgcgagaagtacggcatccccaaggaccgcagccacatcatcggccacaaccaggtgcccggcgccacccacaccgacccgggctccaactggaactggaccacgtacatgaactacgtgaccggtggcggcggcaccccctcgtggaccaccacgatcgacaacgccacctccggccagttcaccgccagcgccaactggggcacctccacctactccagccagcgctacggctccgactaccgcttcgccgacccggtctccgccagcgacgcggcgtggtactcggccgccatccccagcgcgggcacctaccgcgtcgaggtctggtatccggccgacgcgggctacaacagctcggcgccgtacatcgtggcgacgtcaggcggcaaccagaccgtctacgtcgaccaacgcagcggcggcggctcctggaagagcatcggcacgttctcgctgaacgcggggacgtacaacgtggtcggaatcagccggtggaccgccggcaccggctacgtcatcgccgacgccgtccgcatcagccgcgtctga

SEQ ID NO: 20 - protein, 510 aa, Nonomuraea coxensis
MELARTRLTALATSFLAVLVLVATGRPALAAPTAADGTLTAAFAKAAAAYDVPRDLLVALGYAETHLDGHDGKPSASGGYGVMHLVSNPTNHSLEKAAELTGRSTGELRADDAANVLGGAAVLRSHADALGLDETARKDAGRWYQAVAKYGNATSPELARLYADAVYESLGLGIDIRGVQVKPQEVTADRGEYAKARDLNAKADAGVLSTDYGPAAWVAASSSNYTASSRPSSYAIDRVIIHVTQGSYAGTISWFQNPSAQVSAHYVVKSSNGAITQMVREKDVAWHAGNWTYNTRSIGIEHEGYVNDASWFTDAMYRASAALTKNICEKYGIPKDRSHIIGHNQVPGATHTDPGSNWNWTTYMNYVTGGGGTPSWTTTIDNATSGQFTASANWGTSTYSSQRYGSDYRFADPVSASDAAWYSAAIPSAGTYRVEVWYPADAGYNSSAPYIVATSGGNQTVYVDQRSGGGSWKSIGTFSLNAGTYNVVGISRWTAGTGYVIADAVRISRV

SEQ ID NO: 21 - protein, 480 aa, Nonomuraea coxensis
APTAADGTLTAAFAKAAAAYDVPRDLLVALGYAETHLDGHDGKPSASGGYGVMHLVSNPTNHSLEKAAELTGRSTGELRADDAANVLGGAAVLRSHADALGLDETARKDAGRWYQAVAKYGNATSPELARLYADAVYESLGLGIDIRGVQVKPQEVTADRGEYAKARDLNAKADAGVLSTDYGPAAWVAASSSNYTASSRPSSYAIDRVIIHVTQGSYAGTISWFQNPSAQVSAHYVVKSSNGAITQMVREKDVAWHAGNWTYNTRSIGIEHEGYVNDASWFTDAMYRASAALTKNICEKYGIPKDRSHIIGHNQVPGATHTDPGSNWNWTTYMNYVTGGGGTPSWTTTIDNATSGQFTASANWGTSTYSSQRYGSDYRFADPVSASDAAWYSAAIPSAGTYRVEVWYPADAGYNSSAPYIVATSGGNQTVYVDQRSGGGSWKSIGTFSLNAGTYNVVGISRWTAGTGYVIADAVRISRV

SEQ ID NO: 22 - DNA, 1548 bp, Micromonospora fulvopurpurea
  sig_peptide 1..93
  mat_peptide 94..1548
  source 1..1548: mol_type = genomic DNA
  CDS 1..1548
atgacgatccgtcgtccaccacgtcgcgtttcacaccttttaggaggagcaatgattcttatgattggattaactggtcaacctgcacaggctgcgccagctcatggagctcaacctttggctgccgcattcgaccgtgcagctgcgtcatcagatgttcctcgtgacgttcttgcggctcttggatacgcggaaactcgcttggatggtcatggtggtgagccgtctgtatcaggtggatacggtgtaatgcatcttacatcaaacccaaaagttcgtactttagatgaagctgcgcgtcgcacacgtttagaccgtgcagaccttcgtactcgtgacgctgcgaacgtagctggagctgctgcagtacttcgctcttacgcggatgaagcgggtcttacggctgctcagcgcgacgacgtaaaccagtggtatggcccaattgctcgttatggcggaagcactgatgctgcaactgctcgtctttacgcagattctgtatatgacttgcttgcgcgtggattcatcgcgactactgcaggtggcgaagtttctgtagatggccgtccggttgcgcctcaacgtggtcgttatgctgctgtagctccacttggcactggtgatttcggtactctttctactgattacggtccagctgcttgggttccagcgaacagctctaactacacagtttcatctcgtgaatcagcatacccgatcaattacattgttatccatactatgcaaggttcatacgcaggctcaatttcttggtttcagaacgctgctgctggcacatctgctcactaccttcttcgttcttcagatggtgcggttactcaaatggtacgtgacaaagatattgcatggcacgctggcaattggacttacaacactcagtctatcggaatcgagcatgaaggatatgtagacaatgcgtcttggtacacagacgcaatgtatcgttcttctgctgcgcttacacgctacttatgcgacaagtatggaatcccgaaaacacgcactaacattatcggtcataatcaagttcctggagcaactcatactgatccaggccctaactggaactggacgtattacatgcaacttgttactggtggcacgacgccaccgcctacaacatggtctacagttgttgataacacaactgctggtcgcttcactgcatctgctaactggtctacatctacatacagcgctcaacgctatggaactgattatcgttacgcgaatcctgtagctgctagcgatacagcatggtataaggttaacattcctgccacggccacgtatcgtgttgaagtatggtatccagcagtagctggctataacacttctacaccatacatcgtagctacaacaagcggaaaccaaacggtatctgttaaccaaacagctaatggcggtacttggcgttcattaggtactttcacacttgccgctggtgatgcgaacaaggtaggtgtatctcgttggtcaggatctacgggatacgtaatcgccgatgctattcgtgttactcgcgta

SEQ ID NO: 23 - protein, 516 aa, Micromonospora fulvopurpurea
MTIRRPPRRVSHLLGGAMILMIGLTGQPAQAAPAHGAQPLAAAFDRAAASSDVPRDVLAALGYAETRLDGHGGEPSVSGGYGVMHLTSNPKVRTLDEAARRTRLDRADLRTRDAANVAGAAAVLRSYADEAGLTAAQRDDVNQWYGPIARYGGSTDAATARLYADSVYDLLARGFIATTAGGEVSVDGRPVAPQRGRYAAVAPLGTGDFGTLSTDYGPAAWVPANSSNYTVSSRESAYPINYIVIHTMQGSYAGSISWFQNAAAGTSAHYLLRSSDGAVTQMVRDKDIAWHAGNWTYNTQSIGIEHEGYVDNASWYTDAMYRSSAALTRYLCDKYGIPKTRTNIIGHNQVPGATHTDPGPNWNWTYYMQLVTGGTTPPPTTWSTVVDNTTAGRFTASANWSTSTYSAQRYGTDYRYANPVAASDTAWYKVNIPATATYRVEVWYPAVAGYNTSTPYIVATTSGNQTVSVNQTANGGTWRSLGTFTLAAGDANKVGVSRWSGSTGYVIADAIRVTRV

SEQ ID NO: 24 - protein, 485 aa, Micromonospora fulvopurpurea
APAHGAQPLAAAFDRAAASSDVPRDVLAALGYAETRLDGHGGEPSVSGGYGVMHLTSNPKVRTLDEAARRTRLDRADLRTRDAANVAGAAAVLRSYADEAGLTAAQRDDVNQWYGPIARYGGSTDAATARLYADSVYDLLARGFIATTAGGEVSVDGRPVAPQRGRYAAVAPLGTGDFGTLSTDYGPAAWVPANSSNYTVSSRESAYPINYIVIHTMQGSYAGSISWFQNAAAGTSAHYLLRSSDGAVTQMVRDKDIAWHAGNWTYNTQSIGIEHEGYVDNASWYTDAMYRSSAALTRYLCDKYGIPKTRTNIIGHNQVPGATHTDPGPNWNWTYYMQLVTGGTTPPPTTWSTVVDNTTAGRFTASANWSTSTYSAQRYGTDYRYANPVAASDTAWYKVNIPATATYRVEVWYPAVAGYNTSTPYIVATTSGNQTVSVNQTANGGTWRSLGTFTLAAGDANKVGVSRWSGSTGYVIADAIRVTRV

SEQ ID NO: 25 - DNA, 1560 bp, Alicyclobacillus sp.
  sig_peptide 1..84
  mat_peptide 85..1557
  source 1..1560: mol_type = genomic DNA
  CDS 1..1557
atgaagaaaacttgggttacggtcctggctaccaccgcattgaccttctccgtcggcgcaagctacgcaactccctcgtttgcagcgaaatcggacgcgcagtcgcaaacgaccgcaccggcgttggacaatgcgttcaccgcagcggcgaaggaattcaaagtcccgaaagatctgctgatggcgatctcctacgcggaatcccgttggcaagtgccggctgagacggtcgccgatgacgaccatgcgcacggcaaaggcttgatgcacttgacggacaactcgttcaaaaagaacctgagcgacacggcgaaaggtcttgggatcacggcgaagcaactggaggacaacgcagggctgaacatccgcggcggcgcgtacctgctcgccaaagcgcaacatgagctcggcaaaccgctctccgacaacgtcaacgactggtacgaagcggtcgcttcctttgaaggcgcttctgacaagagcgacaaagtgctgttcgccgatgaagtctatcgcatcctgaaggacgggacctcgcttgccatcgacggcggcaccctctcgatcttcccgaacaaaaccgtcgctccggtcaaaggacaactgtcggacgtcacctacggcgtgacgaccaacccgtcggcgaccccggactactccggcgcgatctggaatgcggcgagcacatcgaactaccaagtggcgagccgtccgacttccaacccgatcacctacgtggtcatccacgataccgagggttcctactccggcacgatcaactggttcaaagacccgagcgcgcaagtttcggcacactatgtcgtccgttcttccgacggccagatcacgcagatggtgcaggacaaggacatcgcttggcatgcgcgtaccttcaacacgaacggcatcggcatcgagcatgaaggatatgaagcgcaaaccggttggtacaccgacgcgatgtacacccaatcggcggcgttgacccgtgcgatctgccaaaaatacggcattccgatggaccgcgaccacatcctcggccactccgaactgtggggcaatgaccacaccgatccgggcgtgaactgggattggaacaaatacatgaccaaagtcaccggcgtctccaagaactggaccgtcgtcgacgtcgatgacaaagacacggcatccggcgccttcaccatgtacggcgcttcgcaatactggcacccggtctccggctatggcgtgcacaacgaaatcaactacaccaacggcaacggcgcgacgatctacaactacgcgatctggaagccgacgatcccggtcgccggcaactacgaagtcaaagtgttcgtcccgtccaactacgcgggcacgaccagcgcgaagtatgaaatccactacaacggcggcgtcgtaaccaagaccgtctcgcaaagcgcctactccaaccaatgggtatcgcttggcacctacaacttcgcaaccggcaccggcggctacgtcaagttgggcgacaacaccggcgacaccaacacgatcgccttcgatacgatccgcttcatgggtcaataa

SEQ ID NO: 26 - protein, 519 aa, Alicyclobacillus sp.
MKKTWVTVLATTALTFSVGASYATPSFAAKSDAQSQTTAPALDNAFTAAAKEFKVPKDLLMAISYAESRWQVPAETVADDDHAHGKGLMHLTDNSFKKNLSDTAKGLGITAKQLEDNAGLNIRGGAYLLAKAQHELGKPLSDNVNDWYEAVASFEGASDKSDKVLFADEVYRILKDGTSLAIDGGTLSIFPNKTVAPVKGQLSDVTYGVTTNPSATPDYSGAIWNAASTSNYQVASRPTSNPITYVVIHDTEGSYSGTINWFKDPSAQVSAHYVVRSSDGQITQMVQDKDIAWHARTFNTNGIGIEHEGYEAQTGWYTDAMYTQSAALTRAICQKYGIPMDRDHILGHSELWGNDHTDPGVNWDWNKYMTKVTGVSKNWTVVDVDDKDTASGAFTMYGASQYWHPVSGYGVHNEINYTNGNGATIYNYAIWKPTIPVAGNYEVKVFVPSNYAGTTSAKYEIHYNGGVVTKTVSQSAYSNQWVSLGTYNFATGTGGYVKLGDNTGDTNTIAFDTIRFMGQ

SEQ ID NO: 27 - protein, 491 aa, Alicyclobacillus sp.
AKSDAQSQTTAPALDNAFTAAAKEFKVPKDLLMAISYAESRWQVPAETVADDDHAHGKGLMHLTDNSFKKNLSDTAKGLGITAKQLEDNAGLNIRGGAYLLAKAQHELGKPLSDNVNDWYEAVASFEGASDKSDKVLFADEVYRILKDGTSLAIDGGTLSIFPNKTVAPVKGQLSDVTYGVTTNPSATPDYSGAIWNAASTSNYQVASRPTSNPITYVVIHDTEGSYSGTINWFKDPSAQVSAHYVVRSSDGQITQMVQDKDIAWHARTFNTNGIGIEHEGYEAQTGWYTDAMYTQSAALTRAICQKYGIPMDRDHILGHSELWGNDHTDPGVNWDWNKYMTKVTGVSKNWTVVDVDDKDTASGAFTMYGASQYWHPVSGYGVHNEINYTNGNGATIYNYAIWKPTIPVAGNYEVKVFVPSNYAGTTSAKYEIHYNGGVVTKTVSQSAYSNQWVSLGTYNFATGTGGYVKLGDNTGDTNTIAFDTIRFMGQ

SEQ ID NO: 28 - DNA, 927 bp, Halomonas sp.
  sig_peptide 1..57
  mat_peptide 58..924
  source 1..927: mol_type = genomic DNA
  CDS 1..924
gtgctgcgccattggaccgccgtgatgggcatcgccatggcaagtgcatggctggccggctgcgcctccccggaacacctggagcgacgcgacggctacgtggtggatcacactcacctttcgccgtcgcacaccagccgggtgcgccacttggtgatgcactacaccgacgtcgacgaggccgagtcgctggccgtgctcaccggtcctcacgtcagcgcccactacgtactgccgttgccgccccgggagcgtcgaggcctgccgctggtctatcagctcgtcgacgaggagcgccgcgcctggcacgccggcgccagcgcgtggaaaggccgcccccatatcaacgacacttcgatcggcatcgagatcgtcaataccgggcccgaccgaccctacatcgaggtggagcggttgctggaagggcatcccgaggacgccgtggaagtgaactgggcaccttatccagacgcgcagatcgaggcgctgattgcgctgtcgcgcgacatcatcgagcgccacgacatccaccccaccgacgttgtcgcccactccgacatcgcgccgacgcgcaagatcgaccccggcccacgcttcccctggcgcaaactctaccaggcaggcatcggcgtctggccggaggaggaagcggtatcgcactggcaggctcgcttcgaggcagagcagctaccgctcgccactctgcagcaggcgcttcgcgcctggggctatccgctggaggcgacgggcgagctggaccgccagacccgcgcggtgctacgcgccttccagatgcgcttccggcctgccgactaccgcggcaagccggacgcagagagcgccgccattctctgggcgctgctggaagcgtatcgccccctcgagctggaacggctcgaaggagcgatgacgcagccggaaagctag

SEQ ID NO: 29 - protein, 308 aa, Halomonas sp.
VLRHWTAVMGIAMASAWLAGCASPEHLERRDGYVVDHTHLSPSHTSRVRHLVMHYTDVDEAESLAVLTGPHVSAHYVLPLPPRERRGLPLVYQLVDEERRAWHAGASAWKGRPHINDTSIGIEIVNTGPDRPYIEVERLLEGHPEDAVEVNWAPYPDAQIEALIALSRDIIERHDIHPTDVVAHSDIAPTRKIDPGPRFPWRKLYQAGIGVWPEEEAVSHWQARFEAEQLPLATLQQALRAWGYPLEATGELDRQTRAVLRAFQMRFRPADYRGKPDAESAAILWALLEAYRPLELERLEGAMTQPES

SEQ ID NO: 30 - protein, 289 aa, Halomonas sp.
GCASPEHLERRDGYVVDHTHLSPSHTSRVRHLVMHYTDVDEAESLAVLTGPHVSAHYVLPLPPRERRGLPLVYQLVDEERRAWHAGASAWKGRPHINDTSIGIEIVNTGPDRPYIEVERLLEGHPEDAVEVNWAPYPDAQIEALIALSRDIIERHDIHPTDVVAHSDIAPTRKIDPGPRFPWRKLYQAGIGVWPEEEAVSHWQARFEAEQLPLATLQQALRAWGYPLEATGELDRQTRAVLRAFQMRFRPADYRGKPDAESAAILWALLEAYRPLELERLEGAMTQPES

SEQ ID NO: 31 - DNA, 783 bp, Pseudomonas peli
  sig_peptide 1..45
  mat_peptide 46..780
  source 1..783: mol_type = genomic DNA
  CDS 1..780
atgagaattcttgctcttgccctgctgatcaccctgttgactgcctgcaccagcggcctgcccatcgacacccgctatgaggcgctagggcagaacagccgggtgcagtacatcatcctgcactacacctcgaccaacctgcagcactccctggagctgctgacccagggcgaggtgagcagccattacctgatcggcgagaacccgccgaccatctaccgcctggtggatgagaatcggcgcgcctggcatgccggggtcagtcagtggcagggacgcacctggctcaatggcaccagcatcggtatcgaactggtcaaccagggtttctatgatggccccaatgggcgctactggcagccctatgcgccggcgcagattgatgcgctgatcctcctgctcaaggacatcatgcagcgtcatgagctgcccctgggcagcatcattggtcatagcgatatcgccccccagcgcaaggtcgatccgggcccgttattcccctggcagcgtctggccgaggccggattgataccctggccggaagccggtgcagtggcgcgccagcaggccgtgtacgagcagcaactgcctgatgtggcctggtttcagcagcaactggccagccacggctacgaggtgcccagccatggcgagctggatcaggccacgcgcaatgtgatcgccgccttccagatgaaataccgccaggccaactatgacggcgagccggacagcgaaactggcgccctgctctgggtgctaaacaatagcgccagccgctga

SEQ ID NO: 32 - protein, 260 aa, Pseudomonas peli
MRILALALLITLLTACTSGLPIDTRYEALGQNSRVQYIILHYTSTNLQHSLELLTQGEVSSHYLIGENPPTIYRLVDENRRAWHAGVSQWQGRTWLNGTSIGIELVNQGFYDGPNGRYWQPYAPAQIDALILLLKDIMQRHELPLGSIIGHSDIAPQRKVDPGPLFPWQRLAEAGLIPWPEAGAVARQQAVYEQQLPDVAWFQQQLASHGYEVPSHGELDQATRNVIAAFQMKYRQANYDGEPDSETGALLWVLNNSASR

SEQ ID NO: 33 - protein, 245 aa, Pseudomonas peli
CTSGLPIDTRYEALGQNSRVQYIILHYTSTNLQHSLELLTQGEVSSHYLIGENPPTIYRLVDENRRAWHAGVSQWQGRTWLNGTSIGIELVNQGFYDGPNGRYWQPYAPAQIDALILLLKDIMQRHELPLGSIIGHSDIAPQRKVDPGPLFPWQRLAEAGLIPWPEAGAVARQQAVYEQQLPDVAWFQQQLASHGYEVPSHGELDQATRNVIAAFQMKYRQANYDGEPDSETGALLWVLNNSASR

SEQ ID NO: 34 - DNA, 900 bp, Halomonas sp.
  sig_peptide 1..57
  mat_peptide 58..897
  source 1..900: mol_type = genomic DNA
  CDS 1..897
atgtggcgaaagcgtgttgtcgttgcatccctgaccctgctgctcactgcctgtgcggggcccggccaccgggaacaacgcaatggttatgtggtggaccacacccatgtggcaccttcccacaacagccgagtacggcacctggtgatgcactacacggatgtggatgaagcggagtcgctcgccgtgctcaccggcccccaggtcagcagccactacgtgctgccgctaccggcacgggagcatcgcggccagccgctggtctaccagctcgtcgacgaggagcgccgcgcctggcacgccggggccagcgcctggaagcgccgcaccaatatcaacgatacgtccatcggcatcgagatcgtcaataccggccccgatcgcccctacgccgaggtggagcggctgctggagcagcaccccgaggcgacggtggagatcggttgggcaccctaccccgaggcacagatccaggcgctgatcgccctgtcgcgggatatcatcgagcgccacaatatccatcccaccgacgtggtggcccactcggatatctcgccgacgcgcaagatcgacccgggcccggcgtttccctggcatgccctttacgaagcgggtatcggcgtatggcccgaagaggccaccgtggcgcgctatcgcgaccgcttcgaccaggcgctgcccgagctctccacgctgcaggcggcacttcatgcctggggctatccgctggtggtaagcgacgaactggattcacagactcgcgcggtacttcgcgccttccagatgcgctttcgccccaccgactatcgcggcctgcccgatgccgagaccgccgcaatcctctgggcacttctggcacgctatcgacccgatgaactcaccgcgctggagccccgatag

SEQ ID NO: 35 - protein, 299 aa, Halomonas sp.
MWRKRVVVASLTLLLTACAGPGHREQRNGYVVDHTHVAPSHNSRVRHLVMHYTDVDEAESLAVLTGPQVSSHYVLPLPAREHRGQPLVYQLVDEERRAWHAGASAWKRRTNINDTSIGIEIVNTGPDRPYAEVERLLEQHPEATVEIGWAPYPEAQIQALIALSRDIIERHNIHPTDVVAHSDISPTRKIDPGPAFPWHALYEAGIGVWPEEATVARYRDRFDQALPELSTLQAALHAWGYPLVVSDELDSQTRAVLRAFQMRFRPTDYRGLPDAETAAILWALLARYRPDELTALEPR

SEQ ID NO: 36 - protein, 280 aa, Halomonas sp.
GPGHREQRNGYVVDHTHVAPSHNSRVRHLVMHYTDVDEAESLAVLTGPQVSSHYVLPLPAREHRGQPLVYQLVDEERRAWHAGASAWKRRTNINDTSIGIEIVNTGPDRPYAEVERLLEQHPEATVEIGWAPYPEAQIQALIALSRDIIERHNIHPTDVVAHSDISPTRKIDPGPAFPWHALYEAGIGVWPEEATVARYRDRFDQALPELSTLQAALHAWGYPLVVSDELDSQTRAVLRAFQMRFRPTDYRGLPDAETAAILWALLARYRPDELTALEPR

SEQ ID NO: 37 - DNA, 783 bp, Pseudomonas pseudoalcaligenes
  sig_peptide 1..66
  mat_peptide 67..780
  source 1..783: mol_type = genomic DNA
  CDS 1..780
atgaagtctctttgccttgccctcgcccttgttctgcttgccggttgcaccggtggtctacgtatcgatgacagccacacggcgaccggccagaacagtcgtgtgcaatacgtcgtgctgcactacacctccgctgacctgcagcgctcgctcgacctgctgacgcagaccgaggtgagcagccactacctgatcggggatgcaccaccgaccgtctaccgcctggtggatgagaaccgtcgcgcctggcacgtgggtgtcagcgagtggaaggggcgcacctggctcaacagcaccacgatcggcatcgagctggtcaaccagggctactaccagacgccggccggccgctactggcagcctttcgcgccgcagcagatcgataccctgatcgtgctgctcaaggacatcgtcaagcgtcaccagctaccgctgggctcgatcatcgcgcacagcgatgtggcgccgcagcgcaaggtcgatccgggccctttgttcccctggaagcgtctggccgacgagggcctggtgccctggccgaacgaggacgccgtggcgcgccagcaggcgctgttcagcaccagcctgcccagcgtgcagtggttccaggagcagttggcgcaaaacggctacacggtgccgcagcatggcgagctggatgaggcaacgcgcaatgtcattgccgctttccagatgaaatatcgtccggccaactacgacggccagccggacgccgaaactgcagcgcggttgctggtgctcaatctgcaggcggcaggatag

SEQ ID NO: 38 - protein, 260 aa, Pseudomonas pseudoalcaligenes
MKSLCLALALVLLAGCTGGLRIDDSHTATGQNSRVQYVVLHYTSADLQRSLDLLTQTEVSSHYLIGDAPPTVYRLVDENRRAWHVGVSEWKGRTWLNSTTIGIELVNQGYYQTPAGRYWQPFAPQQIDTLIVLLKDIVKRHQLPLGSIIAHSDVAPQRKVDPGPLFPWKRLADEGLVPWPNEDAVARQQALFSTSLPSVQWFQEQLAQNGYTVPQHGELDEATRNVIAAFQMKYRPANYDGQPDAETAARLLVLNLQAAG

SEQ ID NO: 39 - protein, 238 aa, Pseudomonas pseudoalcaligenes
DDSHTATGQNSRVQYVVLHYTSADLQRSLDLLTQTEVSSHYLIGDAPPTVYRLVDENRRAWHVGVSEWKGRTWLNSTTIGIELVNQGYYQTPAGRYWQPFAPQQIDTLIVLLKDIVKRHQLPLGSIIAHSDVAPQRKVDPGPLFPWKRLADEGLVPWPNEDAVARQQALFSTSLPSVQWFQEQLAQNGYTVPQHGELDEATRNVIAAFQMKYRPANYDGQPDAETAARLLVLNLQAAG

SEQ ID NO: 40 - DNA, 1566 bp, Tumebacillus sp.
  sig_peptide 1..69
  mat_peptide 70..1563
  source 1..1566: mol_type = genomic DNA
  CDS 1..1563
atgaacaaaacgtgggtatcggtcctcgcgaccaccgcactgaccctgtcggtcagcagcgtgtatgcgcaaccaacgcatgcagccaaactcgacaaccaaacggtcgcgacagcaccggttttggaaagcaccttcagctctgctgcgaaggaatttggcgttccgaaggaactgctgatggcgatctcctacagcgaatcgcgctggcagatcgcgccagaggaaacacatctcaccgctgagccggacaaaaacaacggcaaaggcctgatgcacctcaacgacaactccttcaaaaaaggcctgagcgatgctgcaaaagcgctcggcgtctccaagaaacagatggaagacgatgtgcaactgaacatccgcggcggtgcttacctgctcgcgaaggcacaaaaagacctcggcaaggcgcttacatccaacgtcaacgattggtatgaagcggttgcttcctttgaaggtgccaaggacaaggacgttgccgccttgtttgcagatgaagtctaccgtgtgctacaagaaggaaccgcactggcgatcgaaggcggcacgctgaccctcgacccgaactccaaagtcgatccgagcaaaggcgtctacgcaggcttgaccaacggcggcaccaactatggactgactccggactactccggcgcgatctggaacccggcgagcacgtccaactatgccgtcgcctctcgcccgacttcgaacccgatcaactcggtcatcatccatgacaccgagggttcctactccggttcgatcaactggttcaaagacccggcggcacaagtttccgcacactacatcgtccgttcctccgatggccaaatcacccagttggtacaggacaaagacatcgcatggcatgcacgcagcttcaacaccaacggaatcggcatcgaacacgaaggctatgcggcacaaaccggctggtacaccgacgcgatgtacaccgcatcggcggcgctcacccgtgctgtctgcctcaaatacaacatcccgatggaccgcgaccacatcctcgctcactccgaactgtggggcaatgaccacaccgatccgggcgtgaactgggattggaacaaatacatgagcaaagtgaccggtgtgacgaaaaactacgcggcggtactggtcgacaacaccgacgcatcctccggcttcacgctgggtggcccgtcccaatactggcacccgaccgcagggtacggcatccacaaccagatgacgtacacgatgggcaacggcaccaacccgatctccaactacgcgacgtggaagccgacgatcccgactgctggcaactaccaagtcaaagtcttcatcccgtccaacttcgcagcgaccaccaacgcgaagtatgaaatccactacaacggtggcgtcatcaccaaaacgatctcccaagcagcctactccaaccaatgggtgagccttggcacgtacagcttcgcagcaggcaccgcaggctacgtgaaactcggtgacaacaccggcgacacggcgtacgtcggcatcgacggcatgcgtttcctcgctcaataa

SEQ ID NO: 41 - protein, 521 aa, Tumebacillus sp.
MNKTWVSVLATTALTLSVSSVYAQPTHAAKLDNQTVATAPVLESTFSSAAKEFGVPKELLMAISYSESRWQIAPEETHLTAEPDKNNGKGLMHLNDNSFKKGLSDAAKALGVSKKQMEDDVQLNIRGGAYLLAKAQKDLGKALTSNVNDWYEAVASFEGAKDKDVAALFADEVYRVLQEGTALAIEGGTLTLDPNSKVDPSKGVYAGLTNGGTNYGLTPDYSGAIWNPASTSNYAVASRPTSNPINSVIIHDTEGSYSGSINWFKDPAAQVSAHYIVRSSDGQITQLVQDKDIAWHARSFNTNGIGIEHEGYAAQTGWYTDAMYTASAALTRAVCLKYNIPMDRDHILAHSELWGNDHTDPGVNWDWNKYMSKVTGVTKNYAAVLVDNTDASSGFTLGGPSQYWHPTAGYGIHNQMTYTMGNGTNPISNYATWKPTIPTAGNYQVKVFIPSNFAATTNAKYEIHYNGGVITKTISQAAYSNQWVSLGTYSFAAGTAGYVKLGDNTGDTAYVGIDGMRFLAQ

SEQ ID NO: 42 - protein, 498 aa, Tumebacillus sp.
QPTHAAKLDNQTVATAPVLESTFSSAAKEFGVPKELLMAISYSESRWQIAPEETHLTAEPDKNNGKGLMHLNDNSFKKGLSDAAKALGVSKKQMEDDVQLNIRGGAYLLAKAQKDLGKALTSNVNDWYEAVASFEGAKDKDVAALFADEVYRVLQEGTALAIEGGTLTLDPNSKVDPSKGVYAGLTNGGTNYGLTPDYSGAIWNPASTSNYAVASRPTSNPINSVIIHDTEGSYSGSINWFKDPAAQVSAHYIVRSSDGQITQLVQDKDIAWHARSFNTNGIGIEHEGYAAQTGWYTDAMYTASAALTRAVCLKYNIPMDRDHILAHSELWGNDHTDPGVNWDWNKYMSKVTGVTKNYAAVLVDNTDASSGFTLGGPSQYWHPTAGYGIHNQMTYTMGNGTNPISNYATWKPTIPTAGNYQVKVFIPSNFAATTNAKYEIHYNGGVITKTISQAAYSNQWVSLGTYSFAAGTAGYVKLGDNTGDTAYVGIDGMRFLAQ

SEQ ID NO: 43 - DNA, 1506 bp, Pseudomonas pseudoalcaligenes
  sig_peptide 1..75
  mat_peptide 76..1503
  source 1..1506: mol_type = genomic DNA
  CDS 1..1503
atgactcgattagccgctcttgtcgcctcagccctctccgccctcctcatcctcgccggtcagcccgccgccgccgaccgggcgaccccgctcggcgccgccttcgacaaggccgccgccgcccaggacgtcccccgtgacctgctcgccgcgatcgcctacgccgagacccacctcgacggtcacaacggcgagccgagcgccagcggcggctacggtgtgatgcacctggtcagcaaccccaccacgcacaccctggagaaggccgccgagctcacgggcctgcccgcggagaagctgcgcgccgacaccgaggccaacatcctcggcggcgcggccgtcctgcgctccatcgccgacgggctcggcctcgacgaggccgccaggaaggaggagggccgctggtacgaggccgtcgccacgtacggcaacgcctcctcgccggagctggcccgcctctacgccgacgccgtctacgagctgcttggcctcggcttcaaggccaagggcctgcgtgtggccccccgcgaggtcaccgccgacaggggagtgtacgcaggggccagggacctgaacgccaaggactccaacgccctggccgccgccggccctgactacccgaacgccagctgggtgcccgccagttcgagcaactacaccgtgtcgagccgtccttcgagctacgccatcgaccgggtggtcatccacgtgacccagggctcctacgccgggtccatctcctggttccagaacccgagcgcccaggtctccgcgcactacgtgatccgttcctcggacggcgccatcacccagatggtccgtgagaaggacgtcgcatggcacgcgggcaactggagctacaacacccgctccgtcggcatcgagcacgagggcttcgtcaacgacgcctcctggttcaccgacgcgatgtaccgcgcgtccgccgccctgacccgcaacatctgcgacaagtacggcatcccgaaggaccgcagccacatcatcggccacgtggaggtccccggctccacgcacaccgaccccggcccgcactggaactggaacacctacatgtcgtacgtgaccggtggcggcggcggctcgtggtccaccacggtcgacaacaccggcgcgttcaccgcgagcggcaactggggcacttccagctactccgcccagcgctacggcgccgactaccgcttcgccaacccggtggccgccagcgaccccgcctggtaccgggcgaacctgcccagcacgggcagctaccgggtcgaggtctggtacccctccgaccccggctacaacagctcggccccctacatcgtcgccgcctcgggcggcaaccagacggtgtacgtcgaccagcgatccggcggcggtggctggcgcaccatcggcaccttcaccctcaacgccggtgaccgagacgtcgtgggtgtcagccgctggacctcgggcaccggctacgtcgtcgccgacgccgtccgcatcacccgcctgtaa

SEQ ID NO: 44 - protein, 501 aa, Nonomuraea dietziae
MTRLAALVASALSALLILAGQPAAADRATPLGAAFDKAAAAQDVPRDLLAAIAYAETHLDGHNGEPSASGGYGVMHLVSNPTTHTLEKAAELTGLPAEKLRADTEANILGGAAVLRSIADGLGLDEAARKEEGRWYEAVATYGNASSPELARLYADAVYELLGLGFKAKGLRVAPREVTADRGVYAGARDLNAKDSNALAAAGPDYPNASWVPASSSNYTVSSRPSSYAIDRVVIHVTQGSYAGSISWFQNPSAQVSAHYVIRSSDGAITQMVREKDVAWHAGNWSYNTRSVGIEHEGFVNDASWFTDAMYRASAALTRNICDKYGIPKDRSHIIGHVEVPGSTHTDPGPHWNWNTYMSYVTGGGGGSWSTTVDNTGAFTASGNWGTSSYSAQRYGADYRFANPVAASDPAWYRANLPSTGSYRVEVWYPSDPGYNSSAPYIVAASGGNQTVYVDQRSGGGGWRTIGTFTLNAGDRDVVGVSRWTSGTGYVVADAVRITRL

SEQ ID NO: 45 - protein, 476 aa, Nonomuraea dietziae
DRATPLGAAFDKAAAAQDVPRDLLAAIAYAETHLDGHNGEPSASGGYGVMHLVSNPTTHTLEKAAELTGLPAEKLRADTEANILGGAAVLRSIADGLGLDEAARKEEGRWYEAVATYGNASSPELARLYADAVYELLGLGFKAKGLRVAPREVTADRGVYAGARDLNAKDSNALAAAGPDYPNASWVPASSSNYTVSSRPSSYAIDRVVIHVTQGSYAGSISWFQNPSAQVSAHYVIRSSDGAITQMVREKDVAWHAGNWSYNTRSVGIEHEGFVNDASWFTDAMYRASAALTRNICDKYGIPKDRSHIIGHVEVPGSTHTDPGPHWNWNTYMSYVTGGGGGSWSTTVDNTGAFTASGNWGTSSYSAQRYGADYRFANPVAASDPAWYRANLPSTGSYRVEVWYPSDPGYNSSAPYIVAASGGNQTVYVDQRSGGGGWRTIGTFTLNAGDRDVVGVSRWTSGTGYVVADAVRITRL

SEQ ID NO: 46 - DNA, 1509 bp, Laceyella sacchari
  sig_peptide 1..84
  mat_peptide 85..1506
  source 1..1509: mol_type = genomic DNA
  CDS 1..1506
ttgagaaagcgtttgctctcttggttagctatttgctgtctgctcactctgcctttcctttcagctccaccgcccgttgcagccgaggaacagccgtctttgtcccatgtcttcgagatggccgccgcagagtttgaggtgcccgttgaagtgttgttggctattggctatgccgagacgaggtggatggaccatcagggccaacccagccagctcaacggatacggcattatgcatttggccgaaaaccccaccaatgactcattggtacaagccagccgattgctgggcatagataaacaagtgctgacccgggacatccaggctaacatccgtggcgcggcagccgtgttgcagcaaatctccaaagaacaaaaccaggggcaggtgccgaaaaagctggccgattggtacactgttgtagcggaatacagcggactatccagccaacaaaccaaagcatggtatgccgatgatgtatatgctctgatcaaccgcggcgtctctcgtgttatcaacggccaagaggtgcgattagaacccacaccggtcacacccaaccgcggggaatacggtgaacctcgcgatccgtccggccaagccacacccgattatcccgaagcgcgctgggtggcggcatccagtgccaactatactgccgccaacagggaatccgacggcaatgccatcaactacgtgatcatccacaccacgcaaggctcttataatgggacgatcagttggtttcaaaatccctctgcccaagtgagcgcacattatgtcattcgctccagtgatgggcaagtgacccaaatggtgcagaacaaagacatcgcttggcatgcgggcaactgggattacaatgtccactccgtggggatcgagcatgaagggtatgtcaatgatccagcctggtacactgatgccatgtaccgcgcctctgccaagctgacccgctggttgtgcaacaggtacgggattcccaaagaccgcagtcacattatcggccataaccaagttccaggcgccacccacaccgaccccggccccaattgggattggaactattacatgagcttggtcaatcagtcgggcggcggtgccgatttggtcactgataacgccacttcgaaccgcttcactgccagcgccaattgggcgataggcacgaccaatgcccaaaaatacggagcggattaccgctatgccaagcctgaaacgatcagtgacgcagcttggtacaaagtaaacctccccacctctggaagttatgatgtatatgcttggtggccgtcaggctccatctacaatgatcggacaccttatgtgatcaacaccaccagcggtagccagaccgtccacgtttcccaacaatccagcggaggtgtctggaaccatctgggaacctttaactttgcggcgggcgatgccaaccggattgccgtgtcgcgatggacgacaggcaccggttatgtcatcgctgatgctgtcaaattcgtgaaaagatga

SEQ ID NO: 47 - protein, 502 aa, Laceyella sacchari
LRKRLLSWLAICCLLTLPFLSAPPPVAAEEQPSLSHVFEMAAAEFEVPVEVLLAIGYAETRWMDHQGQPSQLNGYGIMHLAENPTNDSLVQASRLLGIDKQVLTRDIQANIRGAAAVLQQISKEQNQGQVPKKLADWYTVVAEYSGLSSQQTKAWYADDVYALINRGVSRVINGQEVRLEPTPVTPNRGEYGEPRDPSGQATPDYPEARWVAASSANYTAANRESDGNAINYVIIHTTQGSYNGTISWFQNPSAQVSAHYVIRSSDGQVTQMVQNKDIAWHAGNWDYNVHSVGIEHEGYVNDPAWYTDAMYRASAKLTRWLCNRYGIPKDRSHIIGHNQVPGATHTDPGPNWDWNYYMSLVNQSGGGADLVTDNATSNRFTASANWAIGTTNAQKYGADYRYAKPETISDAAWYKVNLPTSGSYDVYAWWPSGSIYNDRTPYVINTTSGSQTVHVSQQSSGGVWNHLGTFNFAAGDANRIAVSRWTTGTGYVIADAVKFVKR

SEQ ID NO: 48 - protein, 474 aa, Laceyella sacchari
EEQPSLSHVFEMAAAEFEVPVEVLLAIGYAETRWMDHQGQPSQLNGYGIMHLAENPTNDSLVQASRLLGIDKQVLTRDIQANIRGAAAVLQQISKEQNQGQVPKKLADWYTVVAEYSGLSSQQTKAWYADDVYALINRGVSRVINGQEVRLEPTPVTPNRGEYGEPRDPSGQATPDYPEARWVAASSANYTAANRESDGNAINYVIIHTTQGSYNGTISWFQNPSAQVSAHYVIRSSDGQVTQMVQNKDIAWHAGNWDYNVHSVGIEHEGYVNDPAWYTDAMYRASAKLTRWLCNRYGIPKDRSHIIGHNQVPGATHTDPGPNWDWNYYMSLVNQSGGGADLVTDNATSNRFTASANWAIGTTNAQKYGADYRYAKPETISDAAWYKVNLPTSGSYDVYAWWPSGSIYNDRTPYVINTTSGSQTVHVSQQSSGGVWNHLGTFNFAAGDANRIAVSRWTTGTGYVIADAVKFVKR

SEQ ID NO: 49 - DNA, 1506 bp, Thermostaphylospora chromogena
  sig_peptide 1..87
  mat_peptide 88..1506
  source 1..1506: mol_type = genomic DNA
  CDS 1..1506
atgcgctctcgcttaatggcggtattggcgtgcgctttcacagttttgtttcttactgctgcgccacaagcaggccacgcacgtgctaatcctcttgctgacgcatttgcagaagcggctgcagcgcatgacgttccacgcgaccttttagtagcgcttgcttacgctgaaactcgtttagatgatcacgatggtgaacctagcgctagcggaggatatggtgttatgcatttagtttctaaccctacaaatcactctttagaacgtgcagcagagcttactggcttgcctgttgaaaaacttcgtggcgatacagcagctaacatcatgggtggtgctgctgttttacgtgcgcatgctgatgaattaggtcttgatgaggctgcacgtaaagacccaggtcgttggtacactgctgttgcgcgttatggtggcgcttcagatcctcgtgtagctcgcctttacgcggatgccgtattcgaattgcttggattgggcattgacgcagcaggcgtaactgtagcaccacaagaagtaacggttgaccgtggtgaatacgctgatgttgaagacttaaacgctccaaaagctcgcgttttgtctgcagattatccgcctgctgcatgggtagctgcacacagcagcaattacacggctagctctcgtccttcttcttatgctatcgaccgtgttatcattcacgttactcaaggttcttacgctggtactatctcttggttccaaaacccttcagcgaacgtatctgctcactatgttattcgttcttctgacggagcagtaactcaaatggtacgtgaaaaagatgttgcttggcatgctggaaattggaattacaacacacgctctatcggtattgagcatgaaggttgggttgataacccatcttggttcactgacgctatgtaccgtgcgtctgcagcgcttacacgtcacatttgcgacaagtacggtatcccaaaagaccgtactcatatcattggtcataaccaagttccaggtgcgactcacactgatccgggacctaattgggattggaaccgcttcatggaatacgtaactggtaatggcggaactccgacttggcaggttactgtagataacgctacagctggcaagtttacagcttctgaaaactggggcacttctacatggtcttctcaacgttacggagctgattaccgttttgcgacgccagtacttgcatcagatcctgcatggttccgtgcaacaatcccttctgcgggtgaataccgtatcgaagtttactacccttcagatcctggctacaattcatctactccgtatatcatcgcgacgtcttcaggtaaccgcactgtatatgttgaccagcgttctggtggtggcacgtggcgcagccttggtacttttagccttaatgcaggcgatcagaatgttgttgctgtatcacgttggacatctggaacaggctacgttatcgcggacgctgttcgcatcacacgttac

SEQ ID NO: 50 - protein, 502 aa, Thermostaphylospora chromogena
MRSRLMAVLACAFTVLFLTAAPQAGHARANPLADAFAEAAAAHDVPRDLLVALAYAETRLDDHDGEPSASGGYGVMHLVSNPTNHSLERAAELTGLPVEKLRGDTAANIMGGAAVLRAHADELGLDEAARKDPGRWYTAVARYGGASDPRVARLYADAVFELLGLGIDAAGVTVAPQEVTVDRGEYADVEDLNAPKARVLSADYPPAAWVAAHSSNYTASSRPSSYAIDRVIIHVTQGSYAGTISWFQNPSANVSAHYVIRSSDGAVTQMVREKDVAWHAGNWNYNTRSIGIEHEGWVDNPSWFTDAMYRASAALTRHICDKYGIPKDRTHIIGHNQVPGATHTDPGPNWDWNRFMEYVTGNGGTPTWQVTVDNATAGKFTASENWGTSTWSSQRYGADYRFATPVLASDPAWFRATIPSAGEYRIEVYYPSDPGYNSSTPYIIATSSGNRTVYVDQRSGGGTWRSLGTFSLNAGDQNVVAVSRWTSGTGYVIADAVRITRY

SEQ ID NO: 51 - protein, 473 aa, Thermostaphylospora chromogena
NPLADAFAEAAAAHDVPRDLLVALAYAETRLDDHDGEPSASGGYGVMHLVSNPTNHSLERAAELTGLPVEKLRGDTAANIMGGAAVLRAHADELGLDEAARKDPGRWYTAVARYGGASDPRVARLYADAVFELLGLGIDAAGVTVAPQEVTVDRGEYADVEDLNAPKARVLSADYPPAAWVAAHSSNYTASSRPSSYAIDRVIIHVTQGSYAGTISWFQNPSANVSAHYVIRSSDGAVTQMVREKDVAWHAGNWNYNTRSIGIEHEGWVDNPSWFTDAMYRASAALTRHICDKYGIPKDRTHIIGHNQVPGATHTDPGPNWDWNRFMEYVTGNGGTPTWQVTVDNATAGKFTASENWGTSTWSSQRYGADYRFATPVLASDPAWFRATIPSAGEYRIEVYYPSDPGYNSSTPYIIATSSGNRTVYVDQRSGGGTWRSLGTFSLNAGDQNVVAVSRWTSGTGYVIADAVRITRY

SEQ ID NO: 52 - DNA, 1320 bp, Kribbella aluminosa
  sig_peptide 1..75
  mat_peptide 76..1317
  source 1..1320: mol_type = genomic DNA
  CDS 1..1317
atgtcccgattagcaaagctgtgcgcagccctggccgtcggcgcgctcgcgctgaccgccctcccgagcaacgcggcatcgccacccgccggctctcacctcgccgaggccttcacgaccgccgccgcgaagtacgacgtaccgcgggaagtgctggtcggtgtcggcttcgccgaaacccatctcgacggtcacgacggcacgccgagccaggccaacgggtacggcgtgatgcacctggcgagcaacaacgtgaacaagacgatgtccgaggcaagcaaactgaccggcgtgccggtcgcgaagctgtccaaggacgacgcgtcgaacgtcctcggcgccgccgcggtcctcgactcctacgcggagcaggccaagctcaaagaccgggcagacctcggcaagtggtacggcgtgatcgcgaagtactcgcactccgccgacgcgtcgaccgcacggctctacaccgacgaggtctaccgaatcattgcccggggtgtccgcgccgccggcgtctcgaccgacccgaagccggtcagcccggaccgcggcgcgtacgcgaaggccgctccgctcgggaccgccgccgtcgactacccgagcgcgatctggaacccggcgagcaccagcaactaccgcgtcggccggaccgcggcgatcaccacgatcgtcatccacgtcacccagggctcgtacgccggcaccatcagctggttcaagaacccgtccgcgcaggtcagcgcgcactacgtggtccgctcgagcgacggcgaggtcacccagatggtggccgagaaggacaccgcgtggcacgtccggaccgagaacccgtacacgatcggcatcgagcacgaagggtacgtcgaccagccgtcgtggttcaccgatgcgatgtaccggtcgtcggccgcgctgacccgcaacatcgccgaccggcggggcatcccgaaggaccgggcccacatcaagggccacaacgagatgccgaacaacgaccacaccgacccgggaccgaactggaactgggactactacacgcagctggtgaacggcggcgacccgaacccgccggagtacaacttcaccacctggggtgagggcgtgaacgtccgctcggcgccgaagctgtccgcgtcggtcgtcaccacgcttcccggcccgacccgcgtattcgtcgagtgccaggtgcagggcgacaccgtgacggcgggcggctacaccaacaactggtgggccaagctgcgcgaccagcacgggtacatgaccaacatctacatcgacgacccgaaccagaagctgccaggcgtaccggactgctag

SEQ ID NO: 53 - protein, 439 aa, Kribbella aluminosa
MSRLAKLCAALAVGALALTALPSNAASPPAGSHLAEAFTTAAAKYDVPREVLVGVGFAETHLDGHDGTPSQANGYGVMHLASNNVNKTMSEASKLTGVPVAKLSKDDASNVLGAAAVLDSYAEQAKLKDRADLGKWYGVIAKYSHSADASTARLYTDEVYRIIARGVRAAGVSTDPKPVSPDRGAYAKAAPLGTAAVDYPSAIWNPASTSNYRVGRTAAITTIVIHVTQGSYAGTISWFKNPSAQVSAHYVVRSSDGEVTQMVAEKDTAWHVRTENPYTIGIEHEGYVDQPSWFTDAMYRSSAALTRNIADRRGIPKDRAHIKGHNEMPNNDHTDPGPNWNWDYYTQLVNGGDPNPPEYNFTTWGEGVNVRSAPKLSASVVTTLPGPTRVFVECQVQGDTVTAGGYTNNWWAKLRDQHGYMTNIYIDDPNQKLPGVPDC

SEQ ID NO: 54 - protein, 414 aa, Kribbella aluminosa
ASPPAGSHLAEAFTTAAAKYDVPREVLVGVGFAETHLDGHDGTPSQANGYGVMHLASNNVNKTMSEASKLTGVPVAKLSKDDASNVLGAAAVLDSYAEQAKLKDRADLGKWYGVIAKYSHSADASTARLYTDEVYRIIARGVRAAGVSTDPKPVSPDRGAYAKAAPLGTAAVDYPSAIWNPASTSNYRVGRTAAITTIVIHVTQGSYAGTISWFKNPSAQVSAHYVVRSSDGEVTQMVAEKDTAWHVRTENPYTIGIEHEGYVDQPSWFTDAMYRSSAALTRNIADRRGIPKDRAHIKGHNEMPNNDHTDPGPNWNWDYYTQLVNGGDPNPPEYNFTTWGEGVNVRSAPKLSASVVTTLPGPTRVFVECQVQGDTVTAGGYTNNWWAKLRDQHGYMTNIYIDDPNQKLPGVPDC

SEQ ID NO: 55 - DNA, 1332 bp, Streptomyces griseus
  sig_peptide 1..93
  mat_peptide 94..1329
  source 1..1332: mol_type = genomic DNA
  CDS 1..1329
atgacgtcgaagcagaggatgagactcgcgctcgcactgaccgcggccggggcgctgagcgtggcgctcctctccccggccgcagccggcgcggacaccgtacgaccggaatgcccgcggtccctggcctgcgactgggtccccgcggcctaccagcagaccggagatccggaggacaaggacacctacggcaactacgacacggcgaaccgccccgacagcaacgccgtcaagttcatcgtcctgcacgacaccgaggtcgactacgacaccaccctgaagatcttccagaacccggccaaccagacgtccgcccactacgtggtgcgctcggcggacggccacgtcacacagatggtgaagaacaaggacgtcgcctggcaggcgggcaactggtacctcaacacccactccatcggcatcgagcaggagggcgtcgcagcggagggtgccacgtggtacacctccgagatgtaccggtcgaccgcgcgcctggtgcggtacctggccgcgaagtacgacatcccgctcgaccggcaacacatcctcgggcacgacggtgtgccgcccaccagcgccgccggaacgaagaacatgcactgggacccgggaccctactgggactggaaccgcttcatggcgctactcggcgcgcccaccgcgcccagcgccccgaagcgcagcgaactggtcaccgtcagcgcggacttcgcgaggaaccagcaggagttccgcgactgcgagaagaacgtcgaccttccccggcaggggagcagcgcggtccctctgcacacggagccgtcggcggacgcgcctctcttctccgacccggggctgcatccggacggctcgccggggacgaactgcgccgctgactggggcagcaagatcagtgcgacccagcaggccgtcgtcgccgaccgggtacccggctggacggcgatctggtggtacggggagaaggcttggttcagcactccgcccggtaccagggtcaccaccccgacccgcggcagtgtggtgcggccgaagccgggcacgtccgaagtgctggtgtacggcgtcgcctaccccgagaaggccgagtaccccacggacttcgtcaagccgaccgtgggcacgccgctcgtctacaccatcaaggcgggtcaggccttccccggcggcggcgaggcgcccaccggctactactacgcaccgacgatcgacacctcgaagccgtacgaccacacgtacttcggtggtgcgcagaagtacgtgacggtccagatcggccaccgcatcggcttcgtcaaggcgtccgacgtggacgtggtccgagccggctcatga

SEQ ID NO: 56 - protein, 443 aa, Streptomyces griseus
MTSKQRMRLALALTAAGALSVALLSPAAAGADTVRPECPRSLACDWVPAAYQQTGDPEDKDTYGNYDTANRPDSNAVKFIVLHDTEVDYDTTLKIFQNPANQTSAHYVVRSADGHVTQMVKNKDVAWQAGNWYLNTHSIGIEQEGVAAEGATWYTSEMYRSTARLVRYLAAKYDIPLDRQHILGHDGVPPTSAAGTKNMHWDPGPYWDWNRFMALLGAPTAPSAPKRSELVTVSADFARNQQEFRDCEKNVDLPRQGSSAVPLHTEPSADAPLFSDPGLHPDGSPGTNCAADWGSKISATQQAVVADRVPGWTAIWWYGEKAWFSTPPGTRVTTPTRGSVVRPKPGTSEVLVYGVAYPEKAEYPTDFVKPTVGTPLVYTIKAGQAFPGGGEAPTGYYYAPTIDTSKPYDHTYFGGAQKYVTVQIGHRIGFVKASDVDVVRAGS

SEQ ID NO: 57 - protein, 412 aa, Streptomyces griseus
DTVRPECPRSLACDWVPAAYQQTGDPEDKDTYGNYDTANRPDSNAVKFIVLHDTEVDYDTTLKIFQNPANQTSAHYVVRSADGHVTQMVKNKDVAWQAGNWYLNTHSIGIEQEGVAAEGATWYTSEMYRSTARLVRYLAAKYDIPLDRQHILGHDGVPPTSAAGTKNMHWDPGPYWDWNRFMALLGAPTAPSAPKRSELVTVSADFARNQQEFRDCEKNVDLPRQGSSAVPLHTEPSADAPLFSDPGLHPDGSPGTNCAADWGSKISATQQAVVADRVPGWTAIWWYGEKAWFSTPPGTRVTTPTRGSVVRPKPGTSEVLVYGVAYPEKAEYPTDFVKPTVGTPLVYTIKAGQAFPGGGEAPTGYYYAPTIDTSKPYDHTYFGGAQKYVTVQIGHRIGFVKASDVDVVRAGS

SEQ ID NO: 58 - DNA, 2019 bp, Micromonospora peucetia
  sig_peptide 1..105
  mat_peptide 106..2016
  source 1..2019: mol_type = genomic DNA
  CDS 1..2016
atgcacctgtcagcgacaacgaggagcagacgcgtcctgctggccaccgccgtggccgccgtcatggtggcgacgccgctcaccgccgccggatccgccgccgccgcaccggcgaccgaccggcagcagcagtacgccgccgcggcagccgagtacggcgtgccggagagcgtcctgctcggcgtctcctatctacagtcccgctgggacaccaacgccggcacgccgagcaccagcgccggctacggaccgatgcacctcaccgacgccgagcacgtcgccgccctgcccgggggcacccaccacgacgagggcaccgaggacccgcgcggcgacgactcccgcccgtccctggcggaggcgcgcgagccggccgagccggctcccgccgaggcggcgctccggacgctcgacgccgccgccggactcaccggcgccagcgaggaggcgctgcggacggacgtcacggcgaacatccggggcggcgcggcgctgctggccgcgtaccagaaggagatcggtgccccggtcggtgccgagaccgacccggcggcctggtacggcgcggtggcccgctactccggcgccgacaccaccgacgccgcggcggccttcgccaacgaggtgtacgccaccatcgccacgggcgacacccgactgaccgacgacgggcagcgggtcaccctcgccgcccgcgaggtccagccggagcgttcctggctggaccggctgggcctgcgcaagctcgcccggcccgacgggctggagtgcccgagcgacatctcgtgcgagtggatcccggcgccctaccagaactacggcaccaccctcggcgcgtacggcaaccacgacctggctgaccggcccgcacagcagaagatcgagtacatcgtcatccacgacaccgagggctacttcgggcccagcgtgaacctggtcaaggacccgaagcgggtgggttggcactacaccctgcgctcggtggacggccacatcgcccagcacatcaagaccaagaacgtcggctggcacgccggcaactggtacgtgaactccaagtccatcggccttgagcacgagggcttcgcgggacacggcacctggtacaccgaggcgatgtaccgcacctccgccaagctggtccgccacctggcgcggcagtacaacatcccgctggaccgcaaccacatcatcgggcacgacaacgtccccggcacggtcgccgcgaacgtgcgtggcatgcactgggacgcgggcccgtactgggactggtcgcactacttcgacctgctgaaggcgccgttctggtcgaccggcacgcaccggaccggactggtcaccatcgacccggacttcgccaccaaccagccgcagttcaccggctgcaaccggcagccgccgggcgtgccgaacccgcccccgccgacggctccctgcccgctgcgcggctcgtcggcgctgcccctgcacaacgcgccgagccaggacgcgccgctggtcaacgacatcgcgctgcggcccgacggcaccccgaacaccatgtacgtctccgaccacggcgcccgggtctcggcaggacagacgtacgccctggccgaggtgcggggtgactggacggcgatctggtacctcggccagaaggcgtggttccacaacccggcctcggcgcggaccgccaagtggtccgtcggcctggtcgccaccccgaaggcaggcaagaccaccatcccggtgtacggccgggcgtacccggaggaggcggcctacccggccggcgtgccgtaccagacgatctcgccgctccagtacacgctctcggccggtgagcggtacgccgtcggcaacctgctccccggcgagtactaccgggccaccacgttcgacggctccgccccgggtgaccggaccgtcatccgcggcgagaacaagtacgtgcagatccagttcggccaccgcatcatgtacgtcaacctggccgacgtgaacctgctgccctcgccgctgggcgcgccccgctga

SEQ ID NO: 59 - protein, 672 aa, Micromonospora peucetia
MHLSATTRSRRVLLATAVAAVMVATPLTAAGSAAAAPATDRQQQYAAAAAEYGVPESVLLGVSYLQSRWDTNAGTPSTSAGYGPMHLTDAEHVAALPGGTHHDEGTEDPRGDDSRPSLAEAREPAEPAPAEAALRTLDAAAGLTGASEEALRTDVTANIRGGAALLAAYQKEIGAPVGAETDPAAWYGAVARYSGADTTDAAAAFANEVYATIATGDTRLTDDGQRVTLAAREVQPERSWLDRLGLRKLARPDGLECPSDISCEWIPAPYQNYGTTLGAYGNHDLADRPAQQKIEYIVIHDTEGYFGPSVNLVKDPKRVGWHYTLRSVDGHIAQHIKTKNVGWHAGNWYVNSKSIGLEHEGFAGHGTWYTEAMYRTSAKLVRHLARQYNIPLDRNHIIGHDNVPGTVAANVRGMHWDAGPYWDWSHYFDLLKAPFWSTGTHRTGLVTIDPDFATNQPQFTGCNRQPPGVPNPPPPTAPCPLRGSSALPLHNAPSQDAPLVNDIALRPDGTPNTMYVSDHGARVSAGQTYALAEVRGDWTAIWYLGQKAWFHNPASARTAKWSVGLVATPKAGKTTIPVYGRAYPEEAAYPAGVPYQTISPLQYTLSAGERYAVGNLLPGEYYRATTFDGSAPGDRTVIRGENKYVQIQFGHRIMYVNLADVNLLPSPLGAPR

SEQ ID NO: 60 - protein, 637 aa, Micromonospora peucetia
APATDRQQQYAAAAAEYGVPESVLLGVSYLQSRWDTNAGTPSTSAGYGPMHLTDAEHVAALPGGTHHDEGTEDPRGDDSRPSLAEAREPAEPAPAEAALRTLDAAAGLTGASEEALRTDVTANIRGGAALLAAYQKEIGAPVGAETDPAAWYGAVARYSGADTTDAAAAFANEVYATIATGDTRLTDDGQRVTLAAREVQPERSWLDRLGLRKLARPDGLECPSDISCEWIPAPYQNYGTTLGAYGNHDLADRPAQQKIEYIVIHDTEGYFGPSVNLVKDPKRVGWHYTLRSVDGHIAQHIKTKNVGWHAGNWYVNSKSIGLEHEGFAGHGTWYTEAMYRTSAKLVRHLARQYNIPLDRNHIIGHDNVPGTVAANVRGMHWDAGPYWDWSHYFDLLKAPFWSTGTHRTGLVTIDPDFATNQPQFTGCNRQPPGVPNPPPPTAPCPLRGSSALPLHNAPSQDAPLVNDIALRPDGTPNTMYVSDHGARVSAGQTYALAEVRGDWTAIWYLGQKAWFHNPASARTAKWSVGLVATPKAGKTTIPVYGRAYPEEAAYPAGVPYQTISPLQYTLSAGERYAVGNLLPGEYYRATTFDGSAPGDRTVIRGENKYVQIQFGHRIMYVNLADVNLLPSPLGAPR

SEQ ID NO: 61 - DNA, 1899 bp, Bacillus sp.
  sig_peptide 1..99
  mat_peptide 100..1896
  source 1..1899: mol_type = genomic DNA
  CDS 1..1896
ttgggatttaaaaagctatcatcagccattttaacgttctccttaaccgcaagcctcttggctatccccgctgactttacaccaagcgcaacttctgcagcctcaacagaaaacagcaatgaagcgcaccacctgcaaaaggcttttgaaacagcggcgaaggaatttggagtacctgaatctgtccttctcgccgtcgcttataaccagtcacgctgggagcaccatgaaggccatagtgaggtcggaggctatggcattatgaatcttgcagacttgccagccgacatgagcgccaggggcaagcatgacgacggaatcattgcggctttggataatgaaaatagcatgcttaaaacagccgcaaatcttctaaatgaagatccggaagccttaaaaaaagaccctgaacaaaatatccggggcggggcagctcttttagcagagtttgcacgccaaacgacaggggaacttccttccgatgaagcagactggtacggtgccgtcgttaaatacagcggaacagatcaggaagtcatcgctaaggactttgcagatcaagtctttgagaccattcagcaaggtgctgccagaaaaaaccttgatggacaaagagtcgtattaaacgccaaggaaattacaccaaataagactacagctggcactatccctcttcgcaacaccaaatacacaaataccgactgtccaaatggccttgattgcactttcattcctgctgcttataagcaattttcgagcagcacaagtaattacggcaactacgatatcgccaatcggccaaaggacgatttagatattcgctatattattattcatgatattgagggcacggctgaatccgccatcagccacttccagaatccgtcctacgttagtgcacactatgtcatagattcagagaccggaaaaatcacccagatggtacgtcctgaagatgtgccatggcatgcaggaaactggtattttaatatgcattcgatcggattggagcatgaaggatatgctgcagaaggcgctgactggtacagtgagcaaatgtatcgctctaccgcaaaacttgtaagatacctttcagaccgatttaacattcctttggacagacagcacattatcggccatgatgagatacctggcttaacaacagcaaaacataggagcatgcactgggatccgggcgcttattgggattggggacactttttcgaccttcttggagcttccattaatccaagcagcggagacaaagacagtaatatcgtcacaatccgcccaaatttcaatacaaatcagccagactttacgtatagaggaattaaacaggaacctgagtcttcaagcctgattcatttatacagcgaacccagctttgaggcaccattggtcagtgatcccctgcttcatcctggcggcaccagcacaagaaatattaatgactggggcaataaagctgcgatgggacagagtttctataaagccgggcaggaaggcgattggacagctatttactacgccggccaaaaagcctggttctataatccgaacaataaaaatagcgtcccaggcagcgggaccctcatcacgccaaaagaagggcttgattccatacctgtatatggtgccgcctatcctgacgacgcagcttacgaagaggccggaattgcggaatgggcaagaggaaaagcacaggttctataccagatgccggctggccaaatctatacagcaacagcaccaattcagtccgattactatcatgcgaagtattataatgacccggctacaaataaggttgtaaaggggaatgatgaatactatcagattttttacaatcatcgtctgggattcgtgaagaaaagcgatgtagaagttgtaaattaa

SEQ ID NO: 62 - protein, 632 aa, Bacillus sp.
LGFKKLSSAILTFSLTASLLAIPADFTPSATSAASTENSNEAHHLQKAFETAAKEFGVPESVLLAVAYNQSRWEHHEGHSEVGGYGIMNLADLPADMSARGKHDDGIIAALDNENSMLKTAANLLNEDPEALKKDPEQNIRGGAALLAEFARQTTGELPSDEADWYGAVVKYSGTDQEVIAKDFADQVFETIQQGAARKNLDGQRVVLNAKEITPNKTTAGTIPLRNTKYTNTDCPNGLDCTFIPAAYKQFSSSTSNYGNYDIANRPKDDLDIRYIIIHDIEGTAESAISHFQNPSYVSAHYVIDSETGKITQMVRPEDVPWHAGNWYFNMHSIGLEHEGYAAEGADWYSEQMYRSTAKLVRYLSDRFNIPLDRQHIIGHDEIPGLTTAKHRSMHWDPGAYWDWGHFFDLLGASINPSSGDKDSNIVTIRPNFNTNQPDFTYRGIKQEPESSSLIHLYSEPSFEAPLVSDPLLHPGGTSTRNINDWGNKAAMGQSFYKAGQEGDWTAIYYAGQKAWFYNPNNKNSVPGSGTLITPKEGLDSIPVYGAAYPDDAAYEEAGIAEWARGKAQVLYQMPAGQIYTATAPIQSDYYHAKYYNDPATNKVVKGNDEYYQIFYNHRLGFVKKSDVEVVN

SEQ ID NO: 63 - protein, 599 aa, Bacillus sp.
ASTENSNEAHHLQKAFETAAKEFGVPESVLLAVAYNQSRWEHHEGHSEVGGYGIMNLADLPADMSARGKHDDGIIAALDNENSMLKTAANLLNEDPEALKKDPEQNIRGGAALLAEFARQTTGELPSDEADWYGAVVKYSGTDQEVIAKDFADQVFETIQQGAARKNLDGQRVVLNAKEITPNKTTAGTIPLRNTKYTNTDCPNGLDCTFIPAAYKQFSSSTSNYGNYDIANRPKDDLDIRYIIIHDIEGTAESAISHFQNPSYVSAHYVIDSETGKITQMVRPEDVPWHAGNWYFNMHSIGLEHEGYAAEGADWYSEQMYRSTAKLVRYLSDRFNIPLDRQHIIGHDEIPGLTTAKHRSMHWDPGAYWDWGHFFDLLGASINPSSGDKDSNIVTIRPNFNTNQPDFTYRGIKQEPESSSLIHLYSEPSFEAPLVSDPLLHPGGTSTRNINDWGNKAAMGQSFYKAGQEGDWTAIYYAGQKAWFYNPNNKNSVPGSGTLITPKEGLDSIPVYGAAYPDDAAYEEAGIAEWARGKAQVLYQMPAGQIYTATAPIQSDYYHAKYYNDPATNKVVKGNDEYYQIFYNHRLGFVKKSDVEVVN

SEQ ID NO: 64 - DNA, 1911 bp, Bacillus sporothermodurans
  sig_peptide 1..93
  mat_peptide 94..1908
  source 1..1911: mol_type = genomic DNA
  CDS 1..1908
atgttattacgacgtcttaaaaatcgtacattattgttcacattcatccttatttttagcttacttgttatgccattcggagtatcagaagcaagtaaacattatacgattgatacaaatcatcattctcctttgcagcaagcatttactaaggctgcaaaagagtttcatgtcccagaaagcttattaatgtccgttgcctacaatgagtcacgttggctagatcatcatggtcagccaagcacatcaggtggctatggaatcatgcatttaactgaagccaagccttcacaaagtttgagtgctaaaggaaacggaatagcacattcatctgctattaatgtgcaacaaatgtatacgttgaaaacggcagctaaacttctaggagtaaaggacaatgtgataaaagttaaccccgaacagaacattcgcggtggtgctgcattacttttggaatatgctcgtgatacggttggccatataccgagaagtctcgctgattggtatggggcagttgctaagtatagcggctcaaacaatcaagttgtcgctaatgattttgcagaccaagtgtatgctacgattcaagaaggtgcagaagaggtgttggccgatgggcaacatcttgtgctcaaaccaaataaggtgattccaaataaaaagacaagtgacaaactgaagtttcaaaaaacgaaagaaatggatgtagattgtcctaaaggagtagattgcagatatattccagcagcatataaacaattttctagtcttacagattacggaaattatgatcttgctaccaggccaaaggatggaaatgatattcgttatattattatccacgatactgaaggcagctatgattcggccatcaactggtttcaagatcagtcatatgctagtgctcactatgttattcgttcttccgatggtcaaattacagaaatggtaaaaccggaggatgttgcttggcaagcaggcaactggtactttaatgcacattccatcggaattgagcatgagggttatgcagtccaaggggctacttggtatagtgaacaaatgtatcatgcatctgcaaaactagtaaaatatttagctgaaaaatatcatgttccgttagatcgagcacatattctcggccacgacaacgtccctggtttaacgccagccgcccaaacccgtatgcattgggatcctgcagcttattggaactgggagcattttttcaaaaaactgggagtaccgattcatccaacgaaaggaaagaaaaacagtcggatcgttacgattgcccctaaatatttaaagaacatgccgccattaacttatcaaaatgaacaattagagaagcaaccggcaaatttcgtctatttgcacacggaaccaagtttctctgccccgtacattggtgatccggcacttcatgcggatggatcacctggtacgacagcaatcaatgactggggagataaagcatcgactggacaaagtttctatgtagctgatcataagggtgactggatggcaatctattatggagggaaaaaagcttggtttttcaacccgaaaaggaaaaatacggtatctggaaaaggaattcttgtaacaccaaagaaaggtctcgatgccatccctgtatatggtaccgcctatccagaaaacagcgcttatgaaggaacaggcattcctactgggtcagaagggaaaattacaccgctacaatataccattgcagccggccaagtatatgtcgcaacaaatcctgtaaaagctgactattactatgccaagttatttaatcggctttcagaaaataaagtcgtaaaaggcaatgacgaatactatcaaattttctttaaccaccgtgtcgcatttgtaaagaagagtgatgtagaggtgaaaaagtaa

SEQ ID NO: 65 - protein, 636 aa, Bacillus sporothermodurans
MLLRRLKNRTLLFTFILIFSLLVMPFGVSEASKHYTIDTNHHSPLQQAFTKAAKEFHVPESLLMSVAYNESRWLDHHGQPSTSGGYGIMHLTEAKPSQSLSAKGNGIAHSSAINVQQMYTLKTAAKLLGVKDNVIKVNPEQNIRGGAALLLEYARDTVGHIPRSLADWYGAVAKYSGSNNQVVANDFADQVYATIQEGAEEVLADGQHLVLKPNKVIPNKKTSDKLKFQKTKEMDVDCPKGVDCRYIPAAYKQFSSLTDYGNYDLATRPKDGNDIRYIIIHDTEGSYDSAINWFQDQSYASAHYVIRSSDGQITEMVKPEDVAWQAGNWYFNAHSIGIEHEGYAVQGATWYSEQMYHASAKLVKYLAEKYHVPLDRAHILGHDNVPGLTPAAQTRMHWDPAAYWNWEHFFKKLGVPIHPTKGKKNSRIVTIAPKYLKNMPPLTYQNEQLEKQPANFVYLHTEPSFSAPYIGDPALHADGSPGTTAINDWGDKASTGQSFYVADHKGDWMAIYYGGKKAWFFNPKRKNTVSGKGILVTPKKGLDAIPVYGTAYPENSAYEGTGIPTGSEGKITPLQYTIAAGQVYVATNPVKADYYYAKLFNRLSENKVVKGNDEYYQIFFNHRVAFVKKSDVEVKK

SEQ ID NO: 66 - protein, 605 aa, Bacillus sporothermodurans
SKHYTIDTNHHSPLQQAFTKAAKEFHVPESLLMSVAYNESRWLDHHGQPSTSGGYGIMHLTEAKPSQSLSAKGNGIAHSSAINVQQMYTLKTAAKLLGVKDNVIKVNPEQNIRGGAALLLEYARDTVGHIPRSLADWYGAVAKYSGSNNQVVANDFADQVYATIQEGAEEVLADGQHLVLKPNKVIPNKKTSDKLKFQKTKEMDVDCPKGVDCRYIPAAYKQFSSLTDYGNYDLATRPKDGNDIRYIIIHDTEGSYDSAINWFQDQSYASAHYVIRSSDGQITEMVKPEDVAWQAGNWYFNAHSIGIEHEGYAVQGATWYSEQMYHASAKLVKYLAEKYHVPLDRAHILGHDNVPGLTPAAQTRMHWDPAAYWNWEHFFKKLGVPIHPTKGKKNSRIVTIAPKYLKNMPPLTYQNEQLEKQPANFVYLHTEPSFSAPYIGDPALHADGSPGTTAINDWGDKASTGQSFYVADHKGDWMAIYYGGKKAWFFNPKRKNTVSGKGILVTPKKGLDAIPVYGTAYPENSAYEGTGIPTGSEGKITPLQYTIAAGQVYVATNPVKADYYYAKLFNRLSENKVVKGNDEYYQIFFNHRVAFVKKSDVEVKK

SEQ ID NO: 67 - DNA, 1941 bp, Paenibacillus pini
  sig_peptide 1..108
  mat_peptide 109..1938
  source 1..1941: mol_type = genomic DNA
  CDS 1..1938
ttgaaactactgcgaacaatccaatggaaaaagctgtcgctcgcactcacagtcgcctcactggcggtttccggattcacacccggacttcctgatccattcaaggccgtaccttctgtatatgcggagcaagacaagacgaattcgttgcaacaagccttcgaatcagcggctaaggagttcggagtgcctgtgagcattttgatgtccgtttcctacaaccttacaaggtgggagcatcatcatggccaacccagtacttctggtggatacggtatcatgcatttgaccgacttgccggtacaggataaagaagaccataacggaaccgacgacgaagagaacccatcgacttcggacgatccgagcgttcataccctatctgcggcggcacaactattgaatcttgatccggacttactgaagcaggatcctgtgtgcaacattcgaggcggagccgcattgctggcgaagtacgctcaggaaaccctcggtaaactgacggcatcggaatccgattggtatggtggcgttgccaaatacagcggttctcaagattcgggggcctctctggagtttgccaataatgtatttgatacgattcagcagggaatatcgcggcaaacttcggaaggacagatgctacgtcttccatccaaggaggtgaagcctaacctggataccgttcaaacgcttcatctgcgtccatcgaaacccgataacgtcgagtgccctcgcaatctccactgcagatctgttcctgcagcctatcagcagaacggggatgacccgtcggattattccaattatgatcttgccgaccgtcccaaatttggtcctgatattcgctatattgtcattcatgataccgaagaaacctatcaggatacgctcaatatatttaccaatccgaattcgaatgtcagcgcccactatgttctccgttcgtccgacgggcagatcacgcaaatggtcaagacaaaggatgtaccgtggcatgcaggcaactggtatttcaatatgcactcgattggcgtagagcatgagggctttgctatggaaggagcaacctggttcaccgagaggctttaccgttcatctgcagcattggtacattatctggcggagaaatatgatattccgctcgatcgggcccatattatcggccatgatgaaataccgggtctcactccagcccgtcaaggtgttatgcatcaggatcccggccctttttgggattgggagcactacatggagctagttggagcaccgatccactccaaacatggtactaaagacattgtgacaatcaagcctggctttaaaaccaatcaaccagatatcaatgatgcgcctgctcagccatctaacttcctttacttatataaagagcccgactttaacgctgagctgattgatgatcccgcccttgtctctcaaaacaagaaagacgggttcagcataggagccaaggctaccatagggcaaaccttctcacttgccggcaagcagggggattggaccgcaatctggttcggaggacagaaagcctggttctataatccgaaaggaaaaaatacggtttcaggtaaggggatgctagtcactccgaaagccggcgcagcttccattccggtctatggtgcagcttatccggaagccgcagcatatccagccgatatcacgcctaatgtgctggttccgctgcagtacactatttcgcatggacagtcgtatgttgccgtagaaaaaaacaaaagcgatgattactatgctccggtatacacgaatgacccttatgcgacgaataagctgatcaagagcaaagaagagttctaccgaatatacttcaatcaccgtttcgcctttgtgaaagcctctgatgtcgagaaggtacggaagcagtcggtgaatgaaacaacacgtcaggatatcccataa

SEQ ID NO: 68 - protein, 646 aa, Paenibacillus pini
LKLLRTIQWKKLSLALTVASLAVSGFTPGLPDPFKAVPSVYAEQDKTNSLQQAFESAAKEFGVPVSILMSVSYNLTRWEHHHGQPSTSGGYGIMHLTDLPVQDKEDHNGTDDEENPSTSDDPSVHTLSAAAQLLNLDPDLLKQDPVCNIRGGAALLAKYAQETLGKLTASESDWYGGVAKYSGSQDSGASLEFANNVFDTIQQGISRQTSEGQMLRLPSKEVKPNLDTVQTLHLRPSKPDNVECPRNLHCRSVPAAYQQNGDDPSDYSNYDLADRPKFGPDIRYIVIHDTEETYQDTLNIFTNPNSNVSAHYVLRSSDGQITQMVKTKDVPWHAGNWYFNMHSIGVEHEGFAMEGATWFTERLYRSSAALVHYLAEKYDIPLDRAHIIGHDEIPGLTPARQGVMHQDPGPFWDWEHYMELVGAPIHSKHGTKDIVTIKPGFKTNQPDINDAPAQPSNFLYLYKEPDFNAELIDDPALVSQNKKDGFSIGAKATIGQTFSLAGKQGDWTAIWFGGQKAWFYNPKGKNTVSGKGMLVTPKAGAASIPVYGAAYPEAAAYPADITPNVLVPLQYTISHGQSYVAVEKNKSDDYYAPVYTNDPYATNKLIKSKEEFYRIYFNHRFAFVKASDVEKVRKQSVNETTRQDIP

SEQ ID NO: 69 - protein, 610 aa, Paenibacillus pini
VPSVYAEQDKTNSLQQAFESAAKEFGVPVSILMSVSYNLTRWEHHHGQPSTSGGYGIMHLTDLPVQDKEDHNGTDDEENPSTSDDPSVHTLSAAAQLLNLDPDLLKQDPVCNIRGGAALLAKYAQETLGKLTASESDWYGGVAKYSGSQDSGASLEFANNVFDTIQQGISRQTSEGQMLRLPSKEVKPNLDTVQTLHLRPSKPDNVECPRNLHCRSVPAAYQQNGDDPSDYSNYDLADRPKFGPDIRYIVIHDTEETYQDTLNIFTNPNSNVSAHYVLRSSDGQITQMVKTKDVPWHAGNWYFNMHSIGVEHEGFAMEGATWFTERLYRSSAALVHYLAEKYDIPLDRAHIIGHDEIPGLTPARQGVMHQDPGPFWDWEHYMELVGAPIHSKHGTKDIVTIKPGFKTNQPDINDAPAQPSNFLYLYKEPDFNAELIDDPALVSQNKKDGFSIGAKATIGQTFSLAGKQGDWTAIWFGGQKAWFYNPKGKNTVSGKGMLVTPKAGAASIPVYGAAYPEAAAYPADITPNVLVPLQYTISHGQSYVAVEKNKSDDYYAPVYTNDPYATNKLIKSKEEFYRIYFNHRFAFVKASDVEKVRKQSVNETTRQDIP

SEQ ID NO: 70 - DNA, 711 bp, Bacillus cohnii
  sig_peptide 1..60
  mat_peptide 61..708
  source 1..711: mol_type = genomic DNA
  CDS 1..708
atgaaaatagtagcaacttttttatgtgtatttatttttgtctgtggttgttcaaaggctgaagtcacaaatgtcgagagtgatgagacaatatcattagttaaaaaagaagatgaattaacttataaaaagccagatacaaatccgagtgaatctttatatgtaacttcgtactatttacctaacgataattcgcgacggagaacagcagaagttacacatataatgattcattacaccagtaacgcagcaaggaatccacagaacccgtatgtaatagaagatatttacgcactgtttgaagaatatggcgtttcagcacattatattattgatcgcgaaggtaatatttttcaattagtggatgagagtagagtggcgtttcatgcaggaaaaggaaacgatttaaactttttggactaccgaaataacatgaatgaatattcaattggtatcgaacttatggcaattggaacgaaagaagaaatgagcctaaatttacaggaaggtcaatacgaactaataccaccatcacatataggttatacagatgagcaatatcactcattagcaaaactgttagaagacttgtatgagcgttatccaaaggtattaaaaaacagagagaacgtagtagggcatgatgaatacgcacctgttcgaaaatcagatcctggaagtttattcgattggaataaaattggtttctga

SEQ ID NO: 71 - protein, 236 aa, Bacillus cohnii
MKIVATFLCVFIFVCGCSKAEVTNVESDETISLVKKEDELTYKKPDTNPSESLYVTSYYLPNDNSRRRTAEVTHIMIHYTSNAARNPQNPYVIEDIYALFEEYGVSAHYIIDREGNIFQLVDESRVAFHAGKGNDLNFLDYRNNMNEYSIGIELMAIGTKEEMSLNLQEGQYELIPPSHIGYTDEQYHSLAKLLEDLYERYPKVLKNRENVVGHDEYAPVRKSDPGSLFDWNKIGF

SEQ ID NO: 72 - protein, 216 aa, Bacillus cohnii
EVTNVESDETISLVKKEDELTYKKPDTNPSESLYVTSYYLPNDNSRRRTAEVTHIMIHYTSNAARNPQNPYVIEDIYALFEEYGVSAHYIIDREGNIFQLVDESRVAFHAGKGNDLNFLDYRNNMNEYSIGIELMAIGTKEEMSLNLQEGQYELIPPSHIGYTDEQYHSLAKLLEDLYERYPKVLKNRENVVGHDEYAPVRKSDPGSLFDWNKIGF

SEQ ID NO: 73 - DNA, 1356 bp, Kribbella sp.
  sig_peptide 1..78
  mat_peptide 79..1353
  source 1..1356: mol_type = genomic DNA
  CDS 1..1353
atgagtcacaaagttcccagactggtcgcggtgctcgccgccggcgcactggccttcagcgcgctccccagcaacgcatccacgccggtcgagaccggcagcacctcgacgctgtccgaggccttcaagaccgccgcgacccagtacgacgtaccgcgtgagctcctggtcggcatcgggtacgccgagtcgcacctcgacggccacgacggtcagcccagccaggccaacgggtacggcgtcatgcacctggccagcaacccgagcaacccgaccatgtccgaggcggcgaagctcaccggcctcccggtcgagaagctggccaaggacgagagcgcgaacgtgctcggcgcggccgccgtactcgacgcgtacgccgacaaggccggcctgcaagggcaaacgcgtgacgacatcggcaagtggtacgaggtcgtcgcccagtactcgcactccgccgacggcccgaccgcgcgcctctacaccgacgaggtctaccggatcgtcggcctcggagtcggcgccgaaggcgtctccacccagccggtcaaggtgacgccggaccgcggcaagtacgcgaacgtcgccccgctcggcacccggacgccggcctcgatccaggccgtcgactacccgggcgcgatctggaacgcggccagcaccagcaactaccgcgtcggacgcacctccgcgatcagcacgatcgtcatccacgtgacccaaggctcgtacgccggcacgatcagctggttcaagaacgcgtcggcgcaggtcagcgcgcactacgtggtgcgttccagcgacgggcagatcacccagatggtggccgagaaggacaccgcctggcacgcccgcagcgcgaacccgtactcggtcggcatcgagcacgagggctgggtcgaccagccgtcgtggttcaccgacgcgatgtaccgcgcgtccgcggcgctgacccgcaacatcgccgaccggcgcggcatcccgaagacccggacgtacatcaagggccacagcgagatgcccgacaacgaccacaccgacccgggtccgaactggaactggacctactacatgcagctggtgaacggcagcaacccgaacccgccgacgtacaacttcaccacgtacggcagtggcgtccgggtccgctcggacgcgaagctgaccgcgtccatcgtcaccaccctgcccggcccgacgcaggtcttcgtcacctgccagaagcagggcgacctggtcaccgccgagggcaccagcaacaactggtggtccaagctgcgcgaccagggcggctacatgaccaacatctacatcgaccaccccgacgccaagctcccgggcgtccccgtctgctga

SEQ ID NO: 74 - protein, 451 aa, Kribbella sp.
MSHKVPRLVAVLAAGALAFSALPSNASTPVETGSTSTLSEAFKTAATQYDVPRELLVGIGYAESHLDGHDGQPSQANGYGVMHLASNPSNPTMSEAAKLTGLPVEKLAKDESANVLGAAAVLDAYADKAGLQGQTRDDIGKWYEVVAQYSHSADGPTARLYTDEVYRIVGLGVGAEGVSTQPVKVTPDRGKYANVAPLGTRTPASIQAVDYPGAIWNAASTSNYRVGRTSAISTIVIHVTQGSYAGTISWFKNASAQVSAHYVVRSSDGQITQMVAEKDTAWHARSANPYSVGIEHEGWVDQPSWFTDAMYRASAALTRNIADRRGIPKTRTYIKGHSEMPDNDHTDPGPNWNWTYYMQLVNGSNPNPPTYNFTTYGSGVRVRSDAKLTASIVTTLPGPTQVFVTCQKQGDLVTAEGTSNNWWSKLRDQGGYMTNIYIDHPDAKLPGVPVC

SEQ ID NO: 75 - protein, 425 aa, Kribbella sp.
STPVETGSTSTLSEAFKTAATQYDVPRELLVGIGYAESHLDGHDGQPSQANGYGVMHLASNPSNPTMSEAAKLTGLPVEKLAKDESANVLGAAAVLDAYADKAGLQGQTRDDIGKWYEVVAQYSHSADGPTARLYTDEVYRIVGLGVGAEGVSTQPVKVTPDRGKYANVAPLGTRTPASIQAVDYPGAIWNAASTSNYRVGRTSAISTIVIHVTQGSYAGTISWFKNASAQVSAHYVVRSSDGQITQMVAEKDTAWHARSANPYSVGIEHEGWVDQPSWFTDAMYRASAALTRNIADRRGIPKTRTYIKGHSEMPDNDHTDPGPNWNWTYYMQLVNGSNPNPPTYNFTTYGSGVRVRSDAKLTASIVTTLPGPTQVFVTCQKQGDLVTAEGTSNNWWSKLRDQGGYMTNIYIDHPDAKLPGVPVC

SEQ ID NO: 76 - DNA, 990 bp, Bacillus sp.
  sig_peptide 1..75
  mat_peptide 76..987
  source 1..990: mol_type = genomic DNA
  CDS 1..987
atgaaaaggctcgttcttgtatttagtatcattgccgtactttttatgagcatttctcctgttcatacagttgcagaaaaccatagcaatctgtatgatgtgagatccggagatacattatggaagatcgccaataaatacggtacatctgtccaaaatctaaaagaaacaaatggattgcaatctgatttattgttagttggtcagagattgtttgttccaatgaggtatgaagtcgttgctggagatacactttggaagctttcaagagcatataactcttccgttcaagcgataaaagcaacaaatggacttacatcggatgtattgtacatagggcaaaagttgaaaattcctcctaagaaattacctatggatggtcagtatgttctcatgacgagagaggaatttaaagactggttatttaaccatgaattcacgaggaacataagcctcattcaacagcaccacacgtggtcaccggcttatggacattttaatggcaaaaatcacttttcgttgcttaaggggatggagtattatcatacgaaagaagtgggctgggaaaacattgcccagaacattacaacattcccagacggaaaaatagccgtatctagaccatttaacagtgctcctgacggctcgattggtccaaaggcaaattctgttgggttaaacatcgaacatgttgggaattttgacttaggcaatgatcaaatgactgccgaacatagggaaacgattatctatcttacggcattgctttgtatgaagtttgggttaactccttctgttgacagcatcacatatcatcgttggtgggatatgaacacaaaggagagagtgttggatcgaagtgaaggagtttctgtaaaaacatgcccaggaacgggatttttcggagggaatacaacagaaagtgcaaagaataatttttatcctttagtgtcacgtaaaatgcaagagattagggcatccatgaattaa

SEQ ID NO: 77 - protein, 329 aa, Bacillus sp.
MKRLVLVFSIIAVLFMSISPVHTVAENHSNLYDVRSGDTLWKIANKYGTSVQNLKETNGLQSDLLLVGQRLFVPMRYEVVAGDTLWKLSRAYNSSVQAIKATNGLTSDVLYIGQKLKIPPKKLPMDGQYVLMTREEFKDWLFNHEFTRNISLIQQHHTWSPAYGHFNGKNHFSLLKGMEYYHTKEVGWENIAQNITTFPDGKIAVSRPFNSAPDGSIGPKANSVGLNIEHVGNFDLGNDQMTAEHRETIIYLTALLCMKFGLTPSVDSITYHRWWDMNTKERVLDRSEGVSVKTCPGTGFFGGNTTESAKNNFYPLVSRKMQEIRASMN

SEQ ID NO: 78 - protein, 304 aa, Bacillus sp.
ENHSNLYDVRSGDTLWKIANKYGTSVQNLKETNGLQSDLLLVGQRLFVPMRYEVVAGDTLWKLSRAYNSSVQAIKATNGLTSDVLYIGQKLKIPPKKLPMDGQYVLMTREEFKDWLFNHEFTRNISLIQQHHTWSPAYGHFNGKNHFSLLKGMEYYHTKEVGWENIAQNITTFPDGKIAVSRPFNSAPDGSIGPKANSVGLNIEHVGNFDLGNDQMTAEHRETIIYLTALLCMKFGLTPSVDSITYHRWWDMNTKERVLDRSEGVSVKTCPGTGFFGGNTTESAKNNFYPLVSRKMQEIRASMN

SEQ ID NO: 79 - DNA, 993 bp, Bacillus sp.
  sig_peptide 1..72
  mat_peptide 73..990
  source 1..993: mol_type = genomic DNA
  CDS 1..990
atgaagaaaaaccttgtcctcatttgtagtttaatcctaatcatattcatgaatatccttcctgttcacgcgatttcaaataaccatagcaacctttatgtagtaaaagctggagatacattacctgaaattgcagataaattcgatactaccatagaggagttgaagctaacaaatggtttgcaatccgattctctatttgttgaacaaaaattatgggttcctattatgcatgaagttgttacaggagaaacactgcaggacattgcatcaacttaccattcttcaatagaaaccataaaaaaagcaaatggactcgtttctgatgagctatatgcagggcaaatattaaaagttactcctaaaaaaatgatcatgcaaggtcaacatatccttatgacaaaggaagaatttaaagattggttgtttaacaaccaatttaatcgtgatattcgtatcatccaacaacatcacacatggttaccttcttataagcaatttaaaggtacaaaccattttcaaatgttacaaagtatggagaattttcataagaaggaaatgggctggcataatattgcgcaaaacataacgaccttccctgatggaaaagtagcagtatctagaccatttaacattgccccagaaggttcaattggatcaaaggcgaattcagtagggctaaccatcgaaaatattggtaactttgatctaggtcacgatgtaatgaccaaggaacagcaggatacaattgtttacataactgccttgctttgtatcaagttcggcttaaccccttcaattgacagtattacttatcatcattggtggaatttacaaacaaaggaaagagtattagataacggaccggattataatgtgaagacttgtcccggtactaattttttcggaggcaatgccactaacgatgcaaagaaacacttttaccctcttgtatccgcaaagatagaagaaattgtagcaacaatggattga

SEQ ID NO: 80 - protein, 330 aa, Bacillus sp.
MKKNLVLICSLILIIFMNILPVHAISNNHSNLYVVKAGDTLPEIADKFDTTIEELKLTNGLQSDSLFVEQKLWVPIMHEVVTGETLQDIASTYHSSIETIKKANGLVSDELYAGQILKVTPKKMIMQGQHILMTKEEFKDWLFNNQFNRDIRIIQQHHTWLPSYKQFKGTNHFQMLQSMENFHKKEMGWHNIAQNITTFPDGKVAVSRPFNIAPEGSIGSKANSVGLTIENIGNFDLGHDVMTKEQQDTIVYITALLCIKFGLTPSIDSITYHHWWNLQTKERVLDNGPDYNVKTCPGTNFFGGNATNDAKKHFYPLVSAKIEEIVATMD

SEQ ID NO: 81 - protein, 306 aa, Bacillus sp.
ISNNHSNLYVVKAGDTLPEIADKFDTTIEELKLTNGLQSDSLFVEQKLWVPIMHEVVTGETLQDIASTYHSSIETIKKANGLVSDELYAGQILKVTPKKMIMQGQHILMTKEEFKDWLFNNQFNRDIRIIQQHHTWLPSYKQFKGTNHFQMLQSMENFHKKEMGWHNIAQNITTFPDGKVAVSRPFNIAPEGSIGSKANSVGLTIENIGNFDLGHDVMTKEQQDTIVYITALLCIKFGLTPSIDSITYHHWWNLQTKERVLDNGPDYNVKTCPGTNFFGGNATNDAKKHFYPLVSAKIEEIVATMD

SEQ ID NO: 82 - DNA, 990 bp, Bacillus sp.
  sig_peptide 1..69
  mat_peptide 70..987
  source 1..990: mol_type = genomic DNA
  CDS 1..987
atgaaaaggctcgttcttgtagttagtttaattgccatactttttgtgagcatttctcctgtttctgcagctgccgaaaaccatagcaatctgtatgatgtgagatctggagatacattatggaagatcgccaataaatatggtacatctgtccaaaatttaaaagaaacaaatggactgcaatctgatttgctgttagttggtcaaagattgtttgttccaatgagctacgaagtcgtttctggagatacactttggaagctttcaagagcatataattcttcagtccaagcaataaaagaaacaaatggacttacatcggatgtattgtacatagggcaaaagttaaaaatccctcctaagaaattacctatggatggtcagtatgttctcatgacgcgagaggaatttaaagattggttatttaaccatgaatttacgagaaacataagccttattcaacagcaccacacgtggtcgccggcctatggccattttaatggaaacaatcacttttcgttacttaagggaatggagtattatcatacgaaagaagtgggttgggaaaatatagctcagaaccttacaacattccccgatgggagaatagcagtctctaggccatttaacagtgctccggatggtagtattggaccaaaagctaactcgataggattaaacatcgaacatatcgggaattttgatttaggtaatgatcaaatgacagctgaacatagagaaacgattatctatcttacggcgttgctatgtatgaagttcggattaactccttctattgacagcatcacatatcatcgttggtgggatatgaacacaaaggagcgtgtgttggatcgaagtgaaggagtttctgtgaaaacttgtccaggtactggatttttcggcgggaatacgacagaaagtgctaagagtaatttttatcctttagtgtcacgtaaaatagaagagattagagcaactttgaattaa

SEQ ID NO: 83 - protein, 329 aa, Bacillus sp.
MKRLVLVVSLIAILFVSISPVSAAAENHSNLYDVRSGDTLWKIANKYGTSVQNLKETNGLQSDLLLVGQRLFVPMSYEVVSGDTLWKLSRAYNSSVQAIKETNGLTSDVLYIGQKLKIPPKKLPMDGQYVLMTREEFKDWLFNHEFTRNISLIQQHHTWSPAYGHFNGNNHFSLLKGMEYYHTKEVGWENIAQNLTTFPDGRIAVSRPFNSAPDGSIGPKANSIGLNIEHIGNFDLGNDQMTAEHRETIIYLTALLCMKFGLTPSIDSITYHRWWDMNTKERVLDRSEGVSVKTCPGTGFFGGNTTESAKSNFYPLVSRKIEEIRATLN

SEQ ID NO: 84 - protein, 306 aa, Bacillus sp.
AAENHSNLYDVRSGDTLWKIANKYGTSVQNLKETNGLQSDLLLVGQRLFVPMSYEVVSGDTLWKLSRAYNSSVQAIKETNGLTSDVLYIGQKLKIPPKKLPMDGQYVLMTREEFKDWLFNHEFTRNISLIQQHHTWSPAYGHFNGNNHFSLLKGMEYYHTKEVGWENIAQNLTTFPDGRIAVSRPFNSAPDGSIGPKANSIGLNIEHIGNFDLGNDQMTAEHRETIIYLTALLCMKFGLTPSIDSITYHRWWDMNTKERVLDRSEGVSVKTCPGTGFFGGNTTESAKSNFYPLVSRKIEEIRATLN

SEQ ID NO: 85 - DNA, 2004 bp, Streptomyces sp.
  sig_peptide 1..105
  mat_peptide 106..2001
  source 1..2004: mol_type = genomic DNA
  CDS 1..2001
atgaagaaaccgttggggaaaattgtcgcaagcaccgcactactcatttctgttgcttttagttcatcgatagcatcagcacatcatcatcaccatcatcctagggacgacgacaccggcgtgctccaggcggcgttcgccgacgcggccgagcgctaccaggtgccggaggaagtgctgctcggcgtctcctatctccagtcccgctgggacggccaccgtggcgcggcgagcgtgaccggcggctacggcccgatgcatctgacggacgcgcacaccgcgctcagccgggaggcgggcgcggtggacaaccaccatctgcacggggaggaggacccgcgcggcgacgacggacgggtccttgagatgccggaggaggagatcccgccggttccggagcgctccgaggtgcccgaacggctccagacggtggaccgggcggcggagctgaccgggctcgacccggaggacctgcggtccagcaacgcggcgaacgtacagggcggcgccgcgctgctggccgcggcgcagcgcgacctcggcctggagccgagcgacgacccggcggactggtacgccgccgtcgcctcctacgcgggctccgcctcgcgggaggcggcgcggttcttcgccgacgaggtgtactcggtgatcaacgagggcgcccggcacaccaccggcgagggccaggtggtggagctgcccgccaccgaggtgaccccgcgcaccgggcaggcgtccgcgctggccctgccggagcagcggcgcgatccccgggtggagtgcccgcccacggtctcctgcgagtggatcccggcggcctacgaggagtacgagcgcgccgacggctcggtgacgtacggcaaccacgacaagggcgaccggccggacgggcagcgggtgcgctacatcgtcatccacgacatggagggctacttctggccgtccatcggactggtgcagaacccgacctgggcgtcctggcagtacagcctccaggcgtcggacgggcacatcgcgcagcacatcctggccaaggacgtcggctggcaggccggcaactggtacgtcaacgccacgtccatcggcctggagcacgagggtttcctgcgggccccggacgcctggtacaccgaggtgatgtaccgctcctcggcgcggctggtgcgctacctggcccggcagcacgacatcccgctggaccggcaccacatcatcggccactacaacgtgccgggcatcggcaccgccaacatccccgggatgcacaccgaccccggtccgtactgggactgggcgcactacttccggctgatgggcgcgccgatcacgcccagcgcccggccgcacagcgcgctggtgacgatccgtcccgactacgacaaccaccgcccggtgttcaccggctgcgacccggcggacgccgccgcgccgtgcgcgccgcacggctccagcgcggtgcggctgcacgtggcgccgagtcatgacgcggcgctggtgccggacatcggcacccatccgggcagcggcggccggtccggcatctcgatctacgacatcggcgcccgcgcctcgaccggccagcagtacgcggtggcggagcgcgccggcgagtggacggcgatctggttcaacggcgagaaggcgtggttccacgacccggcccggcagcggacctcggtcccgagccggggctggatcgccaccccgaaggagggcgtggaccgggtgccggtctacggcgtggcgtacccggagccggaggactacccgccgggcgtgccggtgcgcgcgttcgcgccgctgccgtaccagttcacggccggtcagtcgtacgcggtggggcgcggcggcgaggcgttcgacggggagttctactcggcgacccgcttcgacacggtgggcagccaggtgatccgggggcagcggtactaccagatccagctcggccaccggcacgcgtatgtgaaggcggaggacgtggacgtggtgcccgtgcgctga

SEQ ID NO: 86 - protein, 667 aa, Streptomyces sp.
MKKPLGKIVASTALLISVAFSSSIASAHHHHHHPRDDDTGVLQAAFADAAERYQVPEEVLLGVSYLQSRWDGHRGAASVTGGYGPMHLTDAHTALSREAGAVDNHHLHGEEDPRGDDGRVLEMPEEEIPPVPERSEVPERLQTVDRAAELTGLDPEDLRSSNAANVQGGAALLAAAQRDLGLEPSDDPADWYAAVASYAGSASREAARFFADEVYSVINEGARHTTGEGQVVELPATEVTPRTGQASALALPEQRRDPRVECPPTVSCEWIPAAYEEYERADGSVTYGNHDKGDRPDGQRVRYIVIHDMEGYFWPSIGLVQNPTWASWQYSLQASDGHIAQHILAKDVGWQAGNWYVNATSIGLEHEGFLRAPDAWYTEVMYRSSARLVRYLARQHDIPLDRHHIIGHYNVPGIGTANIPGMHTDPGPYWDWAHYFRLMGAPITPSARPHSALVTIRPDYDNHRPVFTGCDPADAAAPCAPHGSSAVRLHVAPSHDAALVPDIGTHPGSGGRSGISIYDIGARASTGQQYAVAERAGEWTAIWFNGEKAWFHDPARQRTSVPSRGWIATPKEGVDRVPVYGVAYPEPEDYPPGVPVRAFAPLPYQFTAGQSYAVGRGGEAFDGEFYSATRFDTVGSQVIRGQRYYQIQLGHRHAYVKAEDVDVVPVR

SEQ ID NO: 87 - protein, 632 aa, Streptomyces sp.
DDDTGVLQAAFADAAERYQVPEEVLLGVSYLQSRWDGHRGAASVTGGYGPMHLTDAHTALSREAGAVDNHHLHGEEDPRGDDGRVLEMPEEEIPPVPERSEVPERLQTVDRAAELTGLDPEDLRSSNAANVQGGAALLAAAQRDLGLEPSDDPADWYAAVASYAGSASREAARFFADEVYSVINEGARHTTGEGQVVELPATEVTPRTGQASALALPEQRRDPRVECPPTVSCEWIPAAYEEYERADGSVTYGNHDKGDRPDGQRVRYIVIHDMEGYFWPSIGLVQNPTWASWQYSLQASDGHIAQHILAKDVGWQAGNWYVNATSIGLEHEGFLRAPDAWYTEVMYRSSARLVRYLARQHDIPLDRHHIIGHYNVPGIGTANIPGMHTDPGPYWDWAHYFRLMGAPITPSARPHSALVTIRPDYDNHRPVFTGCDPADAAAPCAPHGSSAVRLHVAPSHDAALVPDIGTHPGSGGRSGISIYDIGARASTGQQYAVAERAGEWTAIWFNGEKAWFHDPARQRTSVPSRGWIATPKEGVDRVPVYGVAYPEPEDYPPGVPVRAFAPLPYQFTAGQSYAVGRGGEAFDGEFYSATRFDTVGSQVIRGQRYYQIQLGHRHAYVKAEDVDVVPVR

SEQ ID NO: 88 - DNA, 723 bp, Bacillus sp.
  sig_peptide 1..72
  mat_peptide 73..720
  source 1..723: mol_type = genomic DNA
  CDS 1..720
atggtaagaaacttaggaataaatatactcattgtaaccttatttttaataggatgcacaacagctgaaaattcccaaacgatcggaaaagacacattacaagcaatggaagattcgaagatagagaagaaggacttgatgcttcagtcggcggagacaagaaacgataataatcccgtagattatcttctaccactggaaaactcaaaaccgcggacggaggcaatcacccatgtcatggttcattttataagcaatgctgcgagaaatccagaagatccttataatactattgatatctattccatttttgtggaatatggtgtgtcagcacattatatgattggaagagatgggacagtgtttcggcttgtatcagaagatcgtgtcgcttatcatgctggggcaggagagcttgaagattatcctgactatacggacagcttaaatgaattttcgataggtattgaactccttgctatcggcactagggaagagatgtttcctgctataccagtacgtgtctacaatgtaatagatccaagattagtaggctatacagatgaacaatacgaatcgcttaacgttttattggatgatattttccagagaaacccatcaataaatagagatagaaatcatgtgattgggcatgatgaatacgcacctggaagaaaagctgatccaggctcattatttgattggtctaaactcgggttatag

SEQ ID NO: 89 - protein, 240 aa, Bacillus sp.
MVRNLGINILIVTLFLIGCTTAENSQTIGKDTLQAMEDSKIEKKDLMLQSAETRNDNNPVDYLLPLENSKPRTEAITHVMVHFISNAARNPEDPYNTIDIYSIFVEYGVSAHYMIGRDGTVFRLVSEDRVAYHAGAGELEDYPDYTDSLNEFSIGIELLAIGTREEMFPAIPVRVYNVIDPRLVGYTDEQYESLNVLLDDIFQRNPSINRDRNHVIGHDEYAPGRKADPGSLFDWSKLGL

SEQ ID NO: 90 - protein, 216 aa, Bacillus sp.
SQTIGKDTLQAMEDSKIEKKDLMLQSAETRNDNNPVDYLLPLENSKPRTEAITHVMVHFISNAARNPEDPYNTIDIYSIFVEYGVSAHYMIGRDGTVFRLVSEDRVAYHAGAGELEDYPDYTDSLNEFSIGIELLAIGTREEMFPAIPVRVYNVIDPRLVGYTDEQYESLNVLLDDIFQRNPSINRDRNHVIGHDEYAPGRKADPGSLFDWSKLGL

SEQ ID NO: 91 - DNA, 1002 bp, Bacillus sp.
  sig_peptide 1..81
  mat_peptide 82..999
  source 1..1002: mol_type = genomic DNA
  CDS 1..999
atggttagagggatatgtatagcggtttttatttgttttcttagtatgacaggttttttgaatttaccacaagcccaagcaatagcgcacaatcatagcaacatttatgacatcaaatcaggtgatacattgtggaaaatagctcaaagctatgggacaactgtgaaagatttaaagcaaacgaacggattaacctccgatttacttttaataggtcaaaggttgttcgtacctatgagatatgaagtcgtctctggagatacgctatggaaattgtcacagcaatacaactccacagtaccgtcaatcaaattggcaaacggtttaccatcagacatgatttacataggacaaaagttgaaaatcccacaacgaaagttacgaatggatggccaacatgttttaatgacaaaagaggagtttaggggctggctatttaatcaaaaaatcaaccgtaatatttctatcatccaagaacaccacacttggttaccagattatagtcgctttaatgaaacaaaccatttccaactacttaaaggaatggaatactttcatgtgcatgaaatgggatggagtaacattgcccagaatatcacgaccttcccagatggaacagtggcggtttctcgtccactaaacgtgcctccagacggttctattgggaattacgcgaactctatcggaatcaacattgaaagcgtaggaaatttcgacataggaaacgatcaaatgtcacaggcgcaaaaagaaacaattctctatgttactgctcttctatcaattaaactaggccttacgccatctattgacaccatcacatatcaccactggtgggatatgcgtacaggtaaaagagtgttagacaataacgaaggatattccgtcaaaacatgcccagggacagcattctttggcggcaatagcacaaaaagtgccaaaacaaacttttatccacttgtatctaagaaaatagaggaaatcaagaaaacaatggattag

SEQ ID NO: 92 - protein, 333 aa, Bacillus sp.
MVRGICIAVFICFLSMTGFLNLPQAQAIAHNHSNIYDIKSGDTLWKIAQSYGTTVKDLKQTNGLTSDLLLIGQRLFVPMRYEVVSGDTLWKLSQQYNSTVPSIKLANGLPSDMIYIGQKLKIPQRKLRMDGQHVLMTKEEFRGWLFNQKINRNISIIQEHHTWLPDYSRFNETNHFQLLKGMEYFHVHEMGWSNIAQNITTFPDGTVAVSRPLNVPPDGSIGNYANSIGINIESVGNFDIGNDQMSQAQKETILYVTALLSIKLGLTPSIDTITYHHWWDMRTGKRVLDNNEGYSVKTCPGTAFFGGNSTKSAKTNFYPLVSKKIEEIKKTMD

SEQ ID NO: 93 - protein, 306 aa, Bacillus sp.
IAHNHSNIYDIKSGDTLWKIAQSYGTTVKDLKQTNGLTSDLLLIGQRLFVPMRYEVVSGDTLWKLSQQYNSTVPSIKLANGLPSDMIYIGQKLKIPQRKLRMDGQHVLMTKEEFRGWLFNQKINRNISIIQEHHTWLPDYSRFNETNHFQLLKGMEYFHVHEMGWSNIAQNITTFPDGTVAVSRPLNVPPDGSIGNYANSIGINIESVGNFDIGNDQMSQAQKETILYVTALLSIKLGLTPSIDTITYHHWWDMRTGKRVLDNNEGYSVKTCPGTAFFGGNSTKSAKTNFYPLVSKKIEEIKKTMD

SEQ ID NO: 94 - DNA, 1449 bp, Nonomuraea guangzhouensis
  sig_peptide 1..87
  mat_peptide 88..1446
  source 1..1449: mol_type = genomic DNA
  CDS 1..1446
ttgaaggcccgactccgccattcggccgcgctgctggccggcgcgctcatccccctctcgctgctcgcagggcagccggccggagccgcatccgctgatcccatgagcgacgcgttcgcccgtgcggccaccacgtacgaggtgccccgcgacctgctggtctcgctcgcctacgccgaaacccacctggacggacaccgcggcgaacccagcgcgagcggcggctacggggtgatgcacctggtcagcaaccccgtcaggcacacgctcgagcgcgcggccacgctgacgaagcagcccgtctcggccttgaaggcggacgacgcggccaacatcacgggcggcgccgccgtgctgcgcgcctacgccgacgagctcggactggacgccgcggcccgcaaggacgcgggcaagtggtatcaggcggtggccaagtacggcggcgcctcctcggcggacgtcgcccggttgtacgccgacaccgtctacgaccagctgtcccaagggatcaaggtcaccacgccggccggtgagaccctcacggcgacgccgcggaccgtccagcccgaccgcggctcctacgccaaggcacaggagctgggcaagaccaatacgctcgcggcggcagtggactacccgtcggcggcgtgggcggcggcgcacagcaccaactacgccgtctccaaccgcccgaccagtgacgcgatcgaccgcatcatcatccacgtggcccagggcacgtacgccgggacgatcgactggttccagaccgggcccaggccgaaccccacctcgtcgcactacgtcgtccgttcgtcggacggcgccgtcacccagatggtcagggagaaggaccgggccttccacgcgggcgactccaacaggcgctcggtcggcatcgagcacgagggctgggtcgagcaggcctcctggttcaccgacacgatgtaccgttcctcggccgcgctgacccgcaacatcgccgacaggtacggcatccccaaggaccgcacccacatcatcggccacagcgaggctcccggcgccagccacaccgaccccggtccgaactggaactggaccaagtacatgcagtacgtcaccaacggcagcggtggcggcaccaacccgcacaccgccgagtcggtgtgcggcaccggcttcacggtgatcgactcggcgcccctggggacggcgggcaacgtctacctgacgtacaactcgggcaccggcgccaactgcgtggccacgatcaagctgaccaacctcggcacggccacggccaccagcgcctacctggaggtggagggccagacacgcgtcaccgacagcggtaacttcggctactacgcgggtccggtgcgggccagcgcggccgacaagtgcgtctactggggcggcaaggccggtacggccacctacaacagccccctcgaacactgcgggtaa

SEQ ID NO: 95 - protein, 482 aa, Nonomuraea guangzhouensis
LKARLRHSAALLAGALIPLSLLAGQPAGAASADPMSDAFARAATTYEVPRDLLVSLAYAETHLDGHRGEPSASGGYGVMHLVSNPVRHTLERAATLTKQPVSALKADDAANITGGAAVLRAYADELGLDAAARKDAGKWYQAVAKYGGASSADVARLYADTVYDQLSQGIKVTTPAGETLTATPRTVQPDRGSYAKAQELGKTNTLAAAVDYPSAAWAAAHSTNYAVSNRPTSDAIDRIIIHVAQGTYAGTIDWFQTGPRPNPTSSHYVVRSSDGAVTQMVREKDRAFHAGDSNRRSVGIEHEGWVEQASWFTDTMYRSSAALTRNIADRYGIPKDRTHIIGHSEAPGASHTDPGPNWNWTKYMQYVTNGSGGGTNPHTAESVCGTGFTVIDSAPLGTAGNVYLTYNSGTGANCVATIKLTNLGTATATSAYLEVEGQTRVTDSGNFGYYAGPVRASAADKCVYWGGKAGTATYNSPLEHCG

SEQ ID NO: 96 - protein, 453 aa, Nonomuraea guangzhouensis
ASADPMSDAFARAATTYEVPRDLLVSLAYAETHLDGHRGEPSASGGYGVMHLVSNPVRHTLERAATLTKQPVSALKADDAANITGGAAVLRAYADELGLDAAARKDAGKWYQAVAKYGGASSADVARLYADTVYDQLSQGIKVTTPAGETLTATPRTVQPDRGSYAKAQELGKTNTLAAAVDYPSAAWAAAHSTNYAVSNRPTSDAIDRIIIHVAQGTYAGTIDWFQTGPRPNPTSSHYVVRSSDGAVTQMVREKDRAFHAGDSNRRSVGIEHEGWVEQASWFTDTMYRSSAALTRNIADRYGIPKDRTHIIGHSEAPGASHTDPGPNWNWTKYMQYVTNGSGGGTNPHTAESVCGTGFTVIDSAPLGTAGNVYLTYNSGTGANCVATIKLTNLGTATATSAYLEVEGQTRVTDSGNFGYYAGPVRASAADKCVYWGGKAGTATYNSPLEHCG

SEQ ID NO: 97 - DNA, 1509 bp, Nonomuraea guangzhouensis
  sig_peptide 1..87
  mat_peptide 88..1506
  source 1..1509: mol_type = genomic DNA
  CDS 1..1506
atgtcactctcccccaagcgattaacagctctcgtctcttccgtcctcgccgccctcctggtcttcgccggccagcccgccatcgcggccaaggacacaccgctatccgacgcgttcgcccgcgccgccgcggcccaggacatcccccgcgacctgctcgtcgcgctcgcctacgccgagacccacctggacggccacaacggcgagcccagcgccagcggcggctacggtgtgatgcacctggtcagcaaccccaccacgaaagcgctggagaaggcgtcggggctcaccgggctgccggtcaagaagttgcgcgccgacaccgaggccaacatcctgggcggcgccgcactgctgcgcgccaacgccgacgagctcggcctggacgaggccgccaggaaggaccccggccgctggtacgagtccgtggccaagtacggcaacgccgcctcgccccagctcgcccgcgtctacgccgacgccgtctacgagctgctcggcctcggcatccaggccaaggacgtgcgcgtggcaccgcaagaggtgaccgccgaccgcggcaagtacgccgacacaccctcgctcaaggccgaggtggccagccccgactacccggacgccgcctgggtgcccgcgaactccggcaactacaccgcctccagccgcccgtcgagctacgccatcgaccgcgtgatcattcacgtggcgcagggctcgtacgccgggaccatctcctggttccagaacccgagcgccaacgtctcggcccactacgtggtcaagtcgtcgaacggggccgtcacccagacggtccgcgacaaggacgtcgcctggcacgccggcaactggtcctacaacaccaggtccatcggcatcgagcacgagggcttcgtcaacgaggcctcgtggttcaccgacgcgatgtaccgctcctcggccgcgctgaccaagtacatctgcgacaagtacggcatcccgaaggaccgcacgcacatcatcggccacaaccaggtgccgggcgccacccacaccgacccgggtccgaactggaactggaccacgtacatgaactacgtgaccggtggcggcggcacgccgtcgtggaccaccacggtcgacaacgccacctccggacagttcaccgccagcgcgaactggggcacctccacctactccacccagcgctacggcgcggactaccggttcgccgaccccgtgtccgccagtgacgccgcctggtatcaggcgaccctccccagcgcgggcacctaccgcgtggaggtctggtatccggacgacgccggctacaacagctccgcgccctacatcgtcgccgcctccagcggcaaccagacggtgtacgtggaccaacgctccggcggcggcagctggcacagcttgggcaatttctccctcaacgcgggcaccgccaacgtcgtgggagtcagccggtggacctccggcaccggcctcgtcatcgcggacgccgtccgcatcagcaaggtctga

SEQ ID NO: 98 - protein, 502 aa, Nonomuraea guangzhouensis
MSLSPKRLTALVSSVLAALLVFAGQPAIAAKDTPLSDAFARAAAAQDIPRDLLVALAYAETHLDGHNGEPSASGGYGVMHLVSNPTTKALEKASGLTGLPVKKLRADTEANILGGAALLRANADELGLDEAARKDPGRWYESVAKYGNAASPQLARVYADAVYELLGLGIQAKDVRVAPQEVTADRGKYADTPSLKAEVASPDYPDAAWVPANSGNYTASSRPSSYAIDRVIIHVAQGSYAGTISWFQNPSANVSAHYVVKSSNGAVTQTVRDKDVAWHAGNWSYNTRSIGIEHEGFVNEASWFTDAMYRSSAALTKYICDKYGIPKDRTHIIGHNQVPGATHTDPGPNWNWTTYMNYVTGGGGTPSWTTTVDNATSGQFTASANWGTSTYSTQRYGADYRFADPVSASDAAWYQATLPSAGTYRVEVWYPDDAGYNSSAPYIVAASSGNQTVYVDQRSGGGSWHSLGNFSLNAGTANVVGVSRWTSGTGLVIADAVRISKV

SEQ ID NO: 99 - protein, 473 aa, Nonomuraea guangzhouensis
AKDTPLSDAFARAAAAQDIPRDLLVALAYAETHLDGHNGEPSASGGYGVMHLVSNPTTKALEKASGLTGLPVKKLRADTEANILGGAALLRANADELGLDEAARKDPGRWYESVAKYGNAASPQLARVYADAVYELLGLGIQAKDVRVAPQEVTADRGKYADTPSLKAEVASPDYPDAAWVPANSGNYTASSRPSSYAIDRVIIHVAQGSYAGTISWFQNPSANVSAHYVVKSSNGAVTQTVRDKDVAWHAGNWSYNTRSIGIEHEGFVNEASWFTDAMYRSSAALTKYICDKYGIPKDRTHIIGHNQVPGATHTDPGPNWNWTTYMNYVTGGGGTPSWTTTVDNATSGQFTASANWGTSTYSTQRYGADYRFADPVSASDAAWYQATLPSAGTYRVEVWYPDDAGYNSSAPYIVAASSGNQTVYVDQRSGGGSWHSLGNFSLNAGTANVVGVSRWTSGTGLVIADAVRISKV

SEQ ID NO: 100 - DNA, 711 bp, Bacillus cohnii
  sig_peptide 1..81
  mat_peptide 82..708
  source 1..711: mol_type = genomic DNA
  CDS 1..708
atgaaaaaaatagcaactatcttatgtgtcgttatgttagtcaacggttgttcaaacgttggaatcacaaatgttgagagaaatgagacagtttcattagttaaaaataaagatgaattaacttataaaaagccaaatacaaatccgagtgaatctttacatgtaactccctactatttacctgacgaaaattcgcgacgaagaactgcagaagttacacatgtaatgattcattacacaagtaatgcagcaagaaatccagagaatccgtatgtattagaagacatttactcgctgtttgaagaatatggcgtttctgcacattatattattgatcgggaaggtactatctttcaattagtagatgaaagtagagtagcgtttcatgcaggaaaaggaatggatttaaactacctacaataccgaaatagcatgaatgaatattcaattggtatcgaacttatggcaattggaacaaaggaagaaatgaatttaaatttgcaggaaggtcaatacgaactaataccgccatcctatatagggtatacagatgagcaatatcactcattagcaaaactgttagaagacttgtatgagcgttatccaaaagtattgagaaacagagagaacgtagtagggcatgatgaatacgcacctgttcgaaaatcagaccctggaagtttatttgactgggaaaaaattgggttctga

SEQ ID NO: 101 - protein, 236 aa, Bacillus cohnii
MKKIATILCVVMLVNGCSNVGITNVERNETVSLVKNKDELTYKKPNTNPSESLHVTPYYLPDENSRRRTAEVTHVMIHYTSNAARNPENPYVLEDIYSLFEEYGVSAHYIIDREGTIFQLVDESRVAFHAGKGMDLNYLQYRNSMNEYSIGIELMAIGTKEEMNLNLQEGQYELIPPSYIGYTDEQYHSLAKLLEDLYERYPKVLRNRENVVGHDEYAPVRKSDPGSLFDWEKIGF

SEQ ID NO: 102 - protein, 209 aa, Bacillus cohnii
NETVSLVKNKDELTYKKPNTNPSESLHVTPYYLPDENSRRRTAEVTHVMIHYTSNAARNPENPYVLEDIYSLFEEYGVSAHYIIDREGTIFQLVDESRVAFHAGKGMDLNYLQYRNSMNEYSIGIELMAIGTKEEMNLNLQEGQYELIPPSYIGYTDEQYHSLAKLLEDLYERYPKVLRNRENVVGHDEYAPVRKSDPGSLFDWEKIGF

SEQ ID NO: 103 - DNA, 918 bp, Halomonas sp.
  sig_peptide 1..72
  mat_peptide 73..915
  source 1..918: mol_type = genomic DNA
  CDS 1..915
atggcgcgctttttcgtcgtcactctcgccatgctggtgctgctggccggctgcgccacgccggagcaatacgagcggcgtgacggctatgtggtggaccacacccatgtgtcgccctcccacaacagccgggtacggcatctggtgatgcactacaccgatgtggacgaggccgaatccctggcgaccctgaccggtccccatgtcagcagccactacgtgctgccgctaccggcacgggcacatcgcggcgagccgctggtctaccagctcgtcgacgaggagcgccgcgcctggcacgccggggccagcgcctggaagcgccgcaccaacatcaacgacacctccatcggcatcgagatcgtcaataccggccccgaccgcccctacgccgaggtggagcgggcgctggaggagcacccggaaagcgatcccgccatccactgggcgccctaccccgaggcacagatcgaggcgctgatcgccctgtcgcgggatatcatcacgcgcaacaatattcaccctaccgacgtggtggcccactcggatatctcgccgacgcgcaagatcgacccgggcccggcgtttccctggcatgccctgtacgaagcgggtatcggcgtatggcccgaagcagccaccgtggcacgctatcgcacccgcttcgatcaggcgctgcccgaactcgccaccctgcaggcggcgctccaggcctggggctatccgctggcggtcagcgacgaactggattcacagactcgcgcggtactgcgcgccttccagatgcgctttcgtcccgccgactatcgtggccggcccgacgccgagactgcggcgatcctctgggcactgctggaaagatatcgccccctcgacctggagcggttcgagggggcgatggaagagccggaggcatga

SEQ ID NO: 104 - protein, 305 aa, Halomonas sp.
MARFFVVTLAMLVLLAGCATPEQYERRDGYVVDHTHVSPSHNSRVRHLVMHYTDVDEAESLATLTGPHVSSHYVLPLPARAHRGEPLVYQLVDEERRAWHAGASAWKRRTNINDTSIGIEIVNTGPDRPYAEVERALEEHPESDPAIHWAPYPEAQIEALIALSRDIITRNNIHPTDVVAHSDISPTRKIDPGPAFPWHALYEAGIGVWPEAATVARYRTRFDQALPELATLQAALQAWGYPLAVSDELDSQTRAVLRAFQMRFRPADYRGRPDAETAAILWALLERYRPLDLERFEGAMEEPEA

SEQ ID NO: 105 - protein, 281 aa, Halomonas sp.
ERRDGYVVDHTHVSPSHNSRVRHLVMHYTDVDEAESLATLTGPHVSSHYVLPLPARAHRGEPLVYQLVDEERRAWHAGASAWKRRTNINDTSIGIEIVNTGPDRPYAEVERALEEHPESDPAIHWAPYPEAQIEALIALSRDIITRNNIHPTDVVAHSDISPTRKIDPGPAFPWHALYEAGIGVWPEAATVARYRTRFDQALPELATLQAALQAWGYPLAVSDELDSQTRAVLRAFQMRFRPADYRGRPDAETAAILWALLERYRPLDLERFEGAMEEPEA

SEQ ID NO: 106 - DNA, 1920 bp, Lysobacter capsici
  sig_peptide 1..171
  mat_peptide 172..1917
  source 1..1920: mol_type = genomic DNA
  CDS 1..1917
atgaacgaatattccactgcacgccgtggggtggtgaccggcgtgcgttcgttgtcggggtcgttgaccgtcgctgtgctggcgctcgccgcgccattggccgcgcaggcgcaggccgcgcccgaagaccgcgccctggcacagcacctgcagatcgaggaatcgctgcaacgcgtcgaccgcgcgctgtacgcggactacttccgccaggcctatgcgcgttacccgtcgattccggccggcacgctggaatccatcgcctacgtgatgagccgctggcagcaactgcagcccggctcggtcgcggcctacggcgaacagcaccagcacatgccgcgctcgtacggggtcatgggcttgtaccacggcgaaggcttcgccgatcaagtcggcgaaggcgcgcgcctgatcggcgtgccggccgcgcgcgtgcagcgcgatccgctcagcaacatcctcgcctcggcggccttgctcgatcgcgagttgcgcgccgacgggatcggcgccaagtcggcggtcgaagccacgcgcccggcgctggagcgttacgccggtttcgccggcaatgcgggcaagagtgcgatccaggatcacgcccgttccagtttcgccttcgacgtgttgctggcgcaggacaagggcgtcaacgatcgcggcatcgtcgtgccgatgcgcgcggtcgcctgggaacgcgccttcgacgcgcgcaagctggtgcggctgcgcgcgccgttcgtgcgcctggacgtgagccgcgaccgggtcgaggcgggtagcttgaaggacgacggcgcgttcgcgatcgacccgctcagcgaaaccctgcgcgcgccggcgctgaccgccgccgacgaaaagagcaccgactacggcccggcgctgtgggtcgcctcgccctatcactccgcgcgcacgtcctacgactcggtcaccatccacacgatgcagggttattacgccggcagcatctcctggttccagaacaaccccagcagcgtcagcgcgcattacctgatccgcagttccgacggccagatcacccagatggtgcgcgagaaccgcgcggcccatcacgtcggcgtgcacaacaagaccacgctcggcatcgagcacgaaggtttcatcaacaacgccagctggtacaccgccgcgatgtacaacgcctcggcggcgttgacccggcacttctgcgcgacctacagcgcgatcagctgcgcgagcgcgttcaggggcccggccggcagcggcatcaacgtgctgccggccagcgtcaaggtcaagggccaccagcattacagcagccagacccataccgatccgggcatcaactgggattgggcgcgttactacaacctgctcaacccgggcaatccgcccggcggcggcagcgtgatcgacagtttcgaaagcacggtcgggcatttcgataccggcccggcgtactcgggcagcaccaccggcatcgccgcgacgtcgctgagcgaacgcaactgcaccacgcgcaagaacggcgagtgctcgctgcggctgttgctgaaagacgacgcggccagcgccggcgcctgggcggtgaggctgttgtcgggcagcggcaatccgggcagcaacgcggccttgacccgcgccaacggcaaggtcggcttctgggtcttcaccggcgcgaccggcatgagcgcggcggtcggcatcgacgacagcgacggcaccgagcgttcgatcagccgcgcgatcccggccaacacctggacctacctggagtggagcctgagcgacgacgcgcagtgggatgcgtgggtcggcggcgccaacggcgcgatcaccgccgcgtcggtgaagctcgacgcggtgtggttctaccgcgatcagacctcgttcgatgtgaatgtgtatgtcgacgatgtgcaggtgaagaactga

SEQ ID NO: 107 - protein, 639 aa, Lysobacter capsici
MNEYSTARRGVVTGVRSLSGSLTVAVLALAAPLAAQAQAAPEDRALAQHLQIEESLQRVDRALYADYFRQAYARYPSIPAGTLESIAYVMSRWQQLQPGSVAAYGEQHQHMPRSYGVMGLYHGEGFADQVGEGARLIGVPAARVQRDPLSNILASAALLDRELRADGIGAKSAVEATRPALERYAGFAGNAGKSAIQDHARSSFAFDVLLAQDKGVNDRGIVVPMRAVAWERAFDARKLVRLRAPFVRLDVSRDRVEAGSLKDDGAFAIDPLSETLRAPALTAADEKSTDYGPALWVASPYHSARTSYDSVTIHTMQGYYAGSISWFQNNPSSVSAHYLIRSSDGQITQMVRENRAAHHVGVHNKTTLGIEHEGFINNASWYTAAMYNASAALTRHFCATYSAISCASAFRGPAGSGINVLPASVKVKGHQHYSSQTHTDPGINWDWARYYNLLNPGNPPGGGSVIDSFESTVGHFDTGPAYSGSTTGIAATSLSERNCTTRKNGECSLRLLLKDDAASAGAWAVRLLSGSGNPGSNAALTRANGKVGFWVFTGATGMSAAVGIDDSDGTERSISRAIPANTWTYLEWSLSDDAQWDAWVGGANGAITAASVKLDAVWFYRDQTSFDVNVYVDDVQVKN

SEQ ID NO: 108 - protein, 582 aa, Lysobacter capsici
RVDRALYADYFRQAYARYPSIPAGTLESIAYVMSRWQQLQPGSVAAYGEQHQHMPRSYGVMGLYHGEGFADQVGEGARLIGVPAARVQRDPLSNILASAALLDRELRADGIGAKSAVEATRPALERYAGFAGNAGKSAIQDHARSSFAFDVLLAQDKGVNDRGIVVPMRAVAWERAFDARKLVRLRAPFVRLDVSRDRVEAGSLKDDGAFAIDPLSETLRAPALTAADEKSTDYGPALWVASPYHSARTSYDSVTIHTMQGYYAGSISWFQNNPSSVSAHYLIRSSDGQITQMVRENRAAHHVGVHNKTTLGIEHEGFINNASWYTAAMYNASAALTRHFCATYSAISCASAFRGPAGSGINVLPASVKVKGHQHYSSQTHTDPGINWDWARYYNLLNPGNPPGGGSVIDSFESTVGHFDTGPAYSGSTTGIAATSLSERNCTTRKNGECSLRLLLKDDAASAGAWAVRLLSGSGNPGSNAALTRANGKVGFWVFTGATGMSAAVGIDDSDGTERSISRAIPANTWTYLEWSLSDDAQWDAWVGGANGAITAASVKLDAVWFYRDQTSFDVNVYVDDVQVKN

SEQ ID NO: 109 - protein, 27 aa, Synthetic construct
  REGION 1..27: note = Signal peptide
MKKPLGKIVASTALLISVAFSSSIASA

SEQ ID NO: 110 - protein, 8 aa, Synthetic construct
  REGION 1..8: note = His-TAG
HHHHHHPR

SEQ ID NO: 111 - skipped